(19) United States
(12) Patent Application Publication
(10) Pub. No.: US 2014/0324138 A1
(43) Pub. Date: Oct. 30, 2014

(54) Wirelessly-powered illumination of biological tissue

(71) Applicants: Christian Wentz, Cambridge, MA (US); Jacob Bernstein, Cambridge, MA (US); Edward Boyden, Chestnut Hill, MA (US)

(72) Inventors: Christian Wentz, Cambridge, MA (US); Jacob Bernstein, Cambridge, MA (US); Edward Boyden, Chestnut Hill, MA (US)

(73) Assignee: MASSACHUSETTS INSTITUTE OF TECHNOLOGY, Cambridge, MA (US)

(21) Appl. No.: 14/304,259

(22) Filed: Jun. 13, 2014

Related U.S. Application Data
(63) Continuation-in-part of application No. 13/295,736, filed on Nov. 14, 2011, now abandoned.
Application No. 13/295,736 is a continuation-in-part of application No. 12/355,745, filed on Jan. 16, 2009.
Application No. 12/355,745 is a continuation-in-part of application No. 12/714,436, filed on Feb. 26, 2010.
Application No. 12/714,436 is a continuation-in-part of application No. 13/280,229, filed on Oct. 24, 2011.
Application No. 13/280,229 is a continuation-in-part of application No. 12/118,673, filed on May 9, 2008, now abandoned.
Application No. 13/280,229 is a continuation-in-part of application No. 12/355,745, filed on Jan. 16, 2009.
Application No. 12/355,745 is a continuation-in-part of application No. 12/714,436, filed on Feb. 26, 2010.
Application No. 12/714,436 is a continuation-in-part of application No. 12/843,587, filed on Jul. 26, 2010, now Pat. No. 8,545,543.
(60) Provisional application No. 61/412,954, filed on Nov. 12, 2010.
(60) Provisional application No. 61/021,612, filed on Jan. 16, 2008.
(60) Provisional application No. 61/155,855, filed on Feb. 26, 2009.
(60) Provisional application No. 60/917,055, filed on May 9, 2007.
(60) Provisional application No. 61/249,714, filed on Oct. 8, 2009.
(60) Provisional application No. 61/412,954, filed on Nov. 12, 2010.
(60) Provisional application No. 61/413,161, filed on Nov. 12, 2010.
(60) Provisional application No. 61/413,431, filed on Nov. 13, 2010.
(60) Provisional application No. 61/405,977, filed on Oct. 22, 2010.

(51) Int. Cl. A61N 5/06

(52) U.S. Cl. CPC A61N 5/0622
USPC 607/92; 607/88; 607/90

(57) ABSTRACT

In exemplary implementations of this invention, an implant device is wholly or partially implanted in a mammal. The implant device includes an antenna, circuitry, a supercapacitor, one or more light sources, and an array of optical fibers or light guides. The antenna and circuitry receive energy by wireless transmission from an external transmit coil. The supercapacitor stores at least a portion of the energy and provides power to one or more light sources. The array of optical fibers or light guides deliver light from the light sources to living tissue of a mammal. The tissue includes light-sensitive, heterologously expressed proteins. The light affects the light-sensitive proteins, triggering a change in all or part of the tissue, such as a change in voltage, pH or a change in function.

### RELATED APPLICATIONS

This application is a continuation-in-part of pending U.S. application Ser. No. 13/295,736, filed Nov. 14, 2011, published May 17, 2012, as Publication No. US 2012-0123408 A1 titled “Methods and Apparatus for Wireless Control of Biological Tissue”, which claims the benefit of expired U.S. provisional application Ser. No. 61/412,954, filed Nov. 12, 2010, the entire disclosures of which are herein incorporated by reference.

This application is a continuation-in-part of pending U.S. application Ser. No. 12/355,745, filed Jan. 16, 2009, published Aug. 20, 2009 as Publication No. US 2009-0210039 A1, titled “Prosthetic system for therapeutic optical activation and silencing of genetically-targeted neurons”, which claims the benefit of expired U.S. provisional application Ser. 61/021,612, filed Jan. 16, 2008, the entire disclosures of which are herein incorporated by reference.

This application is a continuation-in-part of pending U.S. application Ser. No. 12/714,436, filed Feb. 26, 2010, published Jul. 7, 2011 as Publication No. US 2011-0165681 A1 titled “Light-Activated Proton Pumps and Applications Thereof”, which claims the benefit of expired U.S. Provisional Application Ser. No. 61/155,855, filed Feb. 26, 2009, the entire disclosures of which are herein incorporated by reference

This application is a continuation-in-part of pending U.S. application Ser. No. 13/280,229, filed Oct. 24, 2011, published Apr. 12, 2012, as Publication No. US 2012-0089205 A1 titled “Methods and Apparatus for High-Throughput Neural Screening” (the “229 Application”).

The 229 application is a continuation-in-part of expired U.S. application Ser. No. 12/118,673, filed May 9, 2008, published Dec. 11, 2008 as Publication No. US 2008-0306576 A1 titled “Optical Cell Control Prosthetics”, which claims the benefit of expired U.S. provisional application Ser. No. 60/917,055, filed May 9, 2007, the entire disclosures of which are herein incorporated by reference.

The 229 Application is also a continuation-in-part of pending U.S. application Ser. No. 12/355/745, filed Jan. 16, 2009, published Aug. 20, 2009 as Publication No. US 2009-0210039 A1, titled “Prosthetic system for therapeutic optical activation and silencing of genetically-targeted neurons”, which claims the benefit of expired U.S. provisional application Ser. 61/021,612, filed Jan. 16, 2008, the entire disclosures of which are herein incorporated by reference.

The 229 Application is also a continuation-in-part of pending U.S. application Ser. No. 12/714,436, filed Feb. 26, 2010, published Jul. 7, 2011 as Publication No. US 2011-0165681 A1 titled “Light-Activated Proton Pumps and Applications Thereof”, which claims the benefit of expired U.S. Provisional Application Ser. No. 61/155,855, filed Feb. 26, 2009, the entire disclosures of which are herein incorporated by reference.

The 229 Application is also a continuation-in-part of pending U.S. application Ser. No. 12/843/587, filed Jul. 26, 2010, published Apr. 14, 2011 as Publication No. US 2011-0087311 A1, titled “Methods and Apparatus for Microstructure Lightguides”, which claims the benefit of expired U.S. Provisional Application Ser. No. 61/249,714, filed Oct. 8, 2009, the entire disclosures of which are herein incorporated by reference.

The 229 Application also claims the benefit of expired U.S. Provisional Application Ser. No. 61/412,954, filed Nov. 12, 2010, the entire disclosure of which is herein incorporated by reference.

The 229 Application also claims the benefit of expired U.S. Provisional Application Ser. No. 61/413,161, filed Nov. 12, 2010, the entire disclosure of which is herein incorporated by reference.

The 229 Application also claims the benefit of expired U.S. Provisional Application Ser. No. 61/413,431, filed Nov. 13, 2010, the entire disclosure of which is herein incorporated by reference.

The 229 Application also claims the benefit of expired U.S. Provisional Application Ser. No. 61/405,977, filed Oct. 22, 2010, the entire disclosure of which is herein incorporated by reference.

As used herein, the “Prior Applications” means the patent applications listed above.

### STATEMENT REGARDING FEDERALLY SPONSORED RESEARCH OR DEVELOPMENT

This invention was made with U.S. government support under Grant Number NIH DP2-OD002002, awarded by the National Institutes of Health (NIH), and Grant Numbers NIH 1RC1MH088182 and NIH 1R43NS070453, each awarded by the National Institute of Health. The government has certain rights in this invention.

### FIELD OF THE TECHNOLOGY

The present invention relates generally to wirelessly-powered apparatus for illuminating light-sensitive biologic tissue, and methods for using the same.

### SUMMARY

In exemplary implementations of this invention, a prosthetic device for optical control of target cells comprises a set of light sources, hardware for guiding light to the target cells, supporting hardware that holds members of the set of light sources with respect to each other and the target cells, control circuitry for controlling the set of light sources, and power circuitry that provides power to the set of light sources and the control circuitry. The device may be wearable or implantable, and may be remotely powered or employ wireless communication. The supporting hardware may comprise implantable hypodermics or cannulas, or a plate or scaffold. The set of light sources may be assembled into an array.

In illustrative implementations, apparatus delivers light to precise locations in intact tissues, in order to optically activate or inactivate specified excitable target cells. The apparatus may comprise set of light sources, accessory hardware for guiding light, supporting hardware to hold members of the set of light sources with respect to each other, the target cells, and external structures, and control and power electronics that monitor target cell state, provide regulated power to the light sources, and communicate data, stimulation protocols, and algorithms. All or part of the apparatus device may be wearable or implantable, and may optionally be remotely powered or employ wireless communication. The set of light sources may be assembled into an array.

In an illustrative implementation, an array of fiber-coupled LED elements are attached to a support. The LED elements are each connected to an optical fiber and a wire. Each wire can run through an optional cannula and are attached to the control circuitry. The target ends of the fibers are aimed to deliver light to specific target cells. In an alternative implementation, the LED is placed at the tip of a hypodermic or cannula and optionally coated by a biocompatible coating.

In illustrative implementations, a supercapacitor-based electronic device delivers high currents to an array of implantable light sources or electrodes. The device receives wireless power from an external transmit coil and receives control signals from either an onboard computer or external wireless data telemetry.

In illustrative implementations, a supercapacitor-based electronic device delivers high currents to an array of implantable light sources or electrodes. The device receives wireless power from an external transmit coil and receives control signals from either an onboard computer or external wireless data telemetry.

In illustrative implementations, this invention may be used to advantage for the optical control of neural tissue. For example, the supercapacitor-based device may deliver high currents to an array of optical fibers or an array of waveguides that is inserted into a mammalian brain, or positioned adjacent to the brain. Each fiber or waveguide in the array is coupled to a light source (LED or laser). The brain has been previously sensitized to light, using genetically encoded optical neural control reagents, which are delivered either using viruses or via transgenic means. The array is used to optically perturb the brain. For example, the neurons of the brain may be activated by one color of light, and/or silenced by another color of light.

In illustrative implementations, this invention may comprise a wirelessly powered and wirelessly controlled headborne system capable of simultaneously driving multiple LEDs and recording neural activity in an awake behaving animal.

A prototype of this headborne system has demonstrated reliable optical stimulation in mice for greater than 3 months while weighing in total approximately 3 grams. This device negates the need for tethered stimulation systems and associated limitations on animal behavior.

In some implementations, an implanted device includes a supercapacitor for energy storage and light sources (e.g., LEDs) for optical control of biological tissue. For example, the device may be cranial implant for optogenetic control. Transcutaneous energy transfer (TET) may be employed to wirelessly deliver energy to the supercapacitor-based implanted device. The external power transmitter for the TET may be battery powered or mains powered.

Alternately, the device for optical control may be external (and located adjacent to or affixed to) the mammal. In that case, wireless TET is not employed. Instead, a wireless power transmitter (using EMF waves) delivers power to the external device. A supercapacitor is used to store energy in the device. Light sources (e.g. LEDs) are housed in this external device.

In some implementations, the device is not used for optical control of biological tissue, but is instead used for electrical stimulation or other EMF-based stimulation or perturbation of biological tissue.

In some implementations, a DC/DC converter circuit in the device is used, after rectification of wirelessly-received energy, to convert higher voltages (e.g., 10-15V open circuit) to lower supercapacitor-safe voltages (e.g., 3-6 V). This makes it easier to avoid a supercapacitor overvoltage condition.

In some implementations, the supercapacitor-based device receives wireless power from an external source continuously or intermittently (more than once every 24 hours). For example, the device may be implanted, and wireless transcutaneous energy transfer (TET) may be used to continuously power the device, without having a secondary storage device (e.g. battery). In that case, a supercapacitor housed in the implant used as a primary storage device.

In some implementations, a DC/DC converter topology before the supercapacitor is used to tune the RF power link open circuit voltage.

In some implementations, a DC/DC converter circuit before the supercapacitor steps down input voltage to a safe voltage for the capacitor (e.g., 5-15 volt input from the power receiver, stepped down to ˜5V for the supercapacitor).

In some implementations, a DC/DC converter circuit after the supercapacitor (that has either a buck/boost or charge pump topology) delivers fixed voltage and/or fixed current from the capacitor over a range of capacitor voltages.

In some implementations of this invention, light output is adaptively controlled by a processor, based at least in part on an algorithm that models heat transfer in tissue (as opposed to merely sensed temperature in tissue).

In some implementations, the processor generates control signals to shutdown light delivery if an increase in tissue temperature (as sensed or modeled) exceeds a specified threshold. This threshold may be programmable, rather than fixed (e.g., rather than a fixed threshold of 1 degree Centigrade). Also, the processor can manage (signal back externally) the state of the capacitor over wireless telemetry. This can be used either onboard or externally for algorithmically optimizing the amount of power to be wirelessly delivered to the device based on the light pulse profile (e.g., transiently increase TET power when higher powers are used by the light sources). In addition, the processor can provide remote updates of state variables (temperature, biosensing, pulse profile, supercapacitor voltage, DC/DC converter input voltage) to the system.

In some implementations, the device is employed for integrated recording of biopotentials using EEG, ECoG, or extracellular electrodes.

The power receiver antenna may be multi-axis. For example, a 3-axis receiver may be used, which allows for power reception regardless of the orientation of the receiver relative to the transmitter.

In some implementations, a multiple unit system is deployed. For example: (a) multiple animals may each have a single capacitor and optical array; (b) a single capacitor may power multiple optical arrays for a single animal, or (c) several units, each with their own capacitor, may run in different locations on a single patient that has multiple needs. In each case, a processor may output control signals to manage the different units that are running simultaneously

In illustrative implementations, an optical prosthesis permits control of neural circuits comprises a probe having a set of light sources, drive circuit connections connected to each light source, a housing surrounding the light sources and drive circuit connections, and drive circuitry for driving and controlling the probe. The drive circuit connections and drive circuitry may optionally provide for wireless communication. The light sources may be light-emitting diodes, lasers, or other suitable sources. The device may optionally include sensors for monitoring the target cells.

In some implementations, a multi-dimensional array of probes is used, each probe having a set of light sources, drive circuit connections connected to each light source, a housing surrounding the light sources and the drive circuit connections, drive circuitry for driving and controlling the probes, and supporting hardware that holds the probes in position with respect to each other and the target cells.

In some implementations, the present invention is an optical prosthesis that can be used to directly remedy aberrant activity in corrupted human brain circuits by controlling neural circuits. It may comprise a scalable, fully-implantable optical prosthetic capable of delivering light of appropriate intensity and wavelength to targeted neurons at arbitrary 3-D locations within the brain, enabling activation and silencing of specific neuron types at multiple locations. In some implementations, the device can be implanted in the brains of animals for neural control

In one aspect, the present invention comprises a set of light sources with optional accessory hardware for guiding light, supporting hardware to hold members of the set of light sources with respect to each other, with respect to the target cells, and perhaps with respect to external structures, and control and drive electronics that provide regulated power to the light sources, communicate data, stimulation protocols, and algorithms to and from the outside world, and optionally monitor target cell state.

In one embodiment, the present invention is a prosthetic device for optical control of target cells, comprising a probe having a set of light sources, drive circuit connections connected to each light source, a housing surrounding the light sources and the drive circuit connections, and drive circuitry for driving and controlling the probe. The drive circuit connections and drive circuitry may optionally provide for wireless communication. The light sources may be light-emitting diodes, lasers, or any other suitable source known in the art. The housing may be a glass capillary tube. The prosthetic device may optionally include sensors for monitoring the target cells.

In another embodiment, the present invention is a prosthetic device for optical control of target cells, comprising an array of probes, each probe having a set of light sources, drive circuit connections connected to each light source, a housing surrounding the light sources and the drive circuit connections, drive circuitry for driving and controlling the probes, and supporting hardware that holds the probes in position with respect to each other and the target cells. The array of probes may be two- or three-dimensional.

In exemplary implementations, the prosthetic device is used to to optically activate or inactivate genetically-specified excitable target cells, such as central nervous system neurons, glia, peripheral neurons, skeletal muscle, smooth muscle, cardiac muscle, pancreatic islet cells, thymus cells, immune cells, or other excitable cells, embedded in intact tissue, such as brain, peripheral nervous system, muscle, and skin, would enable radical new treatments for many disorders (e.g., neuropathic pain, Parkinson's disease, epilepsy, diabetes, and other diseases).

In illustrative implementations, illumination of heterologously expressed light-activated membrane proteins causes these proteins to move ions with spectral selectivity, as well as potential ion-selectivity and cell-type specificity, the latter by way of promoter-targeting.

In exemplary implementations of this invention, an implant device is wholly or partially implanted in a mammal. The implant device includes an antenna, circuitry, a supercapacitor, one or more light sources, and an array of optical fibers or light guides. The antenna and circuitry receive energy by wireless transmission from an external transmit coil. The supercapacitor stores at least a portion of the energy and provides power to one or more light sources. The array of optical fibers or light guides deliver light from the light sources to living tissue of a mammal. The tissue includes light-sensitive, heterologously expressed proteins. The proteins are sensitive to light in a specific wavelength band. The light is in that wavelength band. The light causes a change in a parameter or function of the tissue. For example, the change may be a change in pH or voltage in cells, subcellular regions, or extracellular regions in the tissue, or the change may comprise silencing or activation of a function of the tissue.

In illustrative implementations, neural control technology may be used to silence or activate neural circuit targets. In an illustrative example, a mammalian brain is sensitized to light, using genetically encoded optical neural control reagents, which are delivered either using viruses or via transgenic means. An array of lightguides (e.g., optical fibers or microfabricated waveguides) is inserted into the brain, or positioned adjacent to the brain. Each lightguide is coupled to a light source (e.g., LED or laser). Using this array of lightguides, the brain is optically perturbed. For example, the neurons of the brain may be activated by one color of light, and/or silenced by another color of light.

The ability to optically activate or inactivate genetically-specified excitable target cells, such as central nervous system neurons, glia, peripheral neurons, skeletal muscle, smooth muscle, cardiac muscle, pancreatic islet cells, thymus cells, immune cells, or other excitable cells, embedded in intact tissue, such as brain, peripheral nervous system, muscle, and skin, may enable new treatments for many disorders (e.g., neuropathic pain, Parkinson's disease, epilepsy, diabetes, and other diseases). Molecular-genetic methods for making cells such as neurons sensitive to being activated (e.g., depolarized) or inactivated (e.g., hyperpolarized) by light have been previously developed [X. Han and E. S. Boyden, “Multiple-color optical activation, silencing, and desynchronization of neural activity, with single-spike temporal resolution,” PLoS ONE 2, e299 (2007)], but no method currently exists for delivering light to precise locations in intact tissues.

The description of the present invention in the Summary and Abstract sections hereof is just a summary. It is intended only to give a general introduction to some illustrative implementations of this invention. It does not describe all of the details of this invention. This invention may be implemented in many other ways. Likewise, the description of this invention in the Field of the Technology section is not limiting; instead it identifies, in a general, non-exclusive manner, a field of technology to which exemplary implementations of this invention generally relate.

### BRIEF DESCRIPTION OF THE DRAWINGS

Other aspects, advantages and novel features of the invention will become more apparent from the following detailed description of the invention when considered in conjunction with the accompanying drawings wherein:

FIG. 1 is a comparison graph depicting optical neural silencing by light-driven proton pumping, as revealed by a cross-kingdom functional molecular screen;

FIGS. 2A-I present functional properties of the light-driven proton pump Arch in neurons. In particular, FIGS. 2A and 2B are graphs of the photocurrents of Arch versus Halo measured as a function of light irradiance in patch-clamped cultured neurons for low and high light powers, respectively; FIG. 2C is an action spectrum of Arch measured in cultured neurons by scanning illumination light wavelength through the visible spectrum; FIG. 2D is a histogram depicting the photocurrent of Arch measured as a function of ionic composition; FIG. 2E is a plot of Arch proton photocurrent vs. holding potential; FIG. 2F is a histogram of Trypan blue staining of neurons lentivirally-infected with Arch vs. wild-type neurons; FIGS. 2G, 2H and 2I are histograms of membrane capacitance, membrane resistance, and resting potential in neurons lentivirally-infected with Arch vs. wild-type neurons, respectively;

FIGS. 3A-C graphically depict multicolor silencing of two neural populations, enabled by blue- and red-light drivable ion pumps of different classes. In particular, FIG. 3A is a graph depicting the action spectra of Mac versus Halo; FIG. 3B is is a graph depicting membrane hyperpolarizations elicited by blue versus red light, in cells expressing Halo or Mac; and FIG. 3C graphically depicts action potentials evoked by current injection into patch-clamped cultured neurons transfected with Halo and Mac, that are selectively silenced with red vs. blue light;

FIGS. 4A-G depict results from an experimental implementation of the invention, using high-performance Arch-mediated optical neural silencing of neocortical regions in awake mice. In particular, FIGS. 4A and 4B are fluorescence images showing Arch-GFP expression in the mouse cortex, 1 month after lentiviral injection; FIG. 4C presents four representative extracellular recordings showing neurons undergoing 5-s, 15-s and 1-min periods of light illumination; FIG. 4D presents a spike raster plot of neural activity in a representative neuron before, during and after 5 s of yellow light illumination, is shown as a spike raster plot, and a histogram of instantaneous firing rate averaged across trials; FIG. 4E presents in vitro data showing, in cultured neurons expressing Arch or eNpHR and receiving trains of somatic current, the percent reduction of spiking under varying light; FIG. 4F is a histogram depicting average change in spike firing during 5 seconds of yellow light illumination and during the 5 seconds immediately after light offset, for the data shown in FIG. 4D; and FIG. 4G is a histogram of percentage reductions in spike rate;

FIGS. 5A, 5B, 5C and 5D depict the temporally precise, reversible, repeatable, and cell type-specific silencing of the non-human primate via Arch and ArchT, respectively;

FIG. 6 is a graph demonstrating that Arch recovers spontaneously in the dark to its original state after prolonged illumination;

FIG. 7 is a graph of intracellular pH measurements in neurons expressing Arch over a 1-min period of continuous illumination and simultaneous imaging, depicting the use of proton pumps to alter intracellular pH using light according to one aspect of the present invention;

FIG. 8 is a graph showing peak current density recorded from wild type leptosphaeria maculans rhodopsin and various mutants;

FIG. 9 is a raw current trace recorded in a HEK cell expressing ArchT(w), illuminated with orange light followed by near-ultraviolet light, demonstrating bi-directional control of membrane voltage using two different colors of light to address one molecule;

FIG. 10 is a plot depicting bi-directional optical control of an archaerhodopsin “W96Y-like” variant derived from Halorubrum strain aus-2, according to one aspect of the present invention;

FIG. 11 is a plot depicting optically induced shunt-like activity exhibited by an archaerhodopsin “W96Y-like” variant derived from Halorubrum strain aus-1, according to one aspect of the present invention;

FIG. 12A is a histogram of photocurrents measured in cultured neurons expressing all known electrogenic archaerhodopsin full sequence clones, demonstrating that the class of proton pumps from halorubrum perform exceptionally well under mammalian physiological conditions;

FIG. 12B is a confocal fluorescence image of cultured neuron expressing Arch with a GFP fused to the C-terminus, showing good membrane localization in the absence of the appended signal sequences;

FIG. 13 is a histogram depicting the cumulative effect of appending signal sequences to a naturally occurring protein sequence;

FIGS. 14A and 14B are fluorescence images showing HEK293 cells transfected with MTS8-GFP and MTS8-ArchT-GFP plasmids, respectively;

FIG. 15 is a trace of the S. sclerotorium opsin in an HEK293 cell;

FIG. 16 is a diagram depicting a fiber-coupled LED element and an array composed of multiple such elements, according to one aspect of the present invention;

FIG. 17 is a diagram depicting a hypodermic LED source, according to another aspect of the present invention;

FIG. 18 is a diagram depicting an embodiment of a plate for holding the circuitry and LEDs, according to another aspect of the present invention;

FIGS. 19A, 19B, 19C and 19D are diagrams depicting four alternative embodiments of an electronics board for operating the fiber array, according to a further aspect of the present invention;

FIG. 20 is a schematic side view of a wirelessly powered and controlled headborne electronics architecture with implanted light sources;

FIG. 21 is a schematic diagram of a headborne/implant device that is powered by an external power transmitter and that employs a data telemetry link;

FIG. 22A is a block diagram of a headborne/implant system;

FIG. 22B is a perspective view of the underside of an optics module;

FIG. 23A is an image depicting neurons optically activated through the light-activated cation channel channelrhodopsin-2 by millisecond-timescale pulses of blue light;

FIG. 23B depicts Poisson trains of spikes elicited by pulses of blue light in two different neurons;

FIG. 23C is an image depicting neurons electrically silenced through light-activated chloride pump halorhodopsin by pulses of yellow light;

FIG. 23D depicts light-driven spike blockades for a representative hippocampal neuron and for a population of neurons, with hyperpolarization induced by periods of yellow light;

FIG. 23E is an image depicting bi-directional control of neural activity by pulses of blue and yellow light, enabling spike-level neural activity control;

FIG. 23F is a graph depicting an action spectrum for channelrhodopsin-2 overlaid with an absorption spectrum for N. pharaonis halorhodopsin;

FIG. 23G depicts hyperpolarization and depolarization events induced in a representative neuron by a Poisson train of alternating pulses of yellow and blue light;

FIG. 24 is an image of neurons in the mouse brain expressing Halo-GFP under the CaMKII promoter, which preferentially labels excitatory neurons; and

FIG. 25 is an experimentally-produced raster plot indicating occurrences of spikes elicited from an excitatory neuron expressing ChR2 in a monkey cortex in response to a brief train of blue light stimulation.

The above Figures show some illustrative implementations of this invention, or provide information that relates to those implementations. However, this invention may be implemented in many other ways.

### DETAILED DESCRIPTION

This invention may be implemented in many different ways. This Detailed Description section sets forth non-limiting examples of how this invention may be implemented.

As used herein, the phrase “illustrative implementation” means an illustrative implementation of the present invention. As used herein, the phrase “in some implementations” means in some implementations of the present invention.

### Optical Control Prosthesis:

In illustrative implementations, a prosthetic device for optical control of target cells comprises a set of light sources, hardware for guiding light to the target cells, supporting hardware that holds members of the set of light sources with respect to each other and the target cells, control circuitry for controlling the set of light sources, and power circuitry that provides power to the set of light sources and the control circuitry. The device may be wearable or implantable, and may be remotely powered or employ wireless communication. The supporting hardware may comprise implantable hypodermics or cannulas, or a plate or scaffold. The set of light sources may be assembled into an array.

In illustrative implementations, a device for delivering light to precise locations in intact tissues. The device invention employs sets of light sources coupled to optical fibers whose ends deliver light to specified groups of target cells within tissue, sets of light sources in hypodermic cannulas that can deliver light locally to specified groups of target cells within tissue, and sets of light sources attached to nerve cuff holding devices that stably bring the light sources into close proximity to a group of target nerve cells. In support of the function of these sets of light sources, the present invention in some aspects includes control and power electronics, which enable battery-powered, wearable, fully implantable, wirelessly-operated, and/or remotely-powered versions of the electronics to drive these light sources, thus enabling the use of these devices as prosthetics. In another aspect, the present invention includes steerable light sources, ways of coupling multiple colors into the same fiber, and other uses of such fibers.

In illustrative implementations, the invention comprises several parts: a set of light sources (such as, but not limited to, LEDs or lasers) with accessory hardware (e.g., fibers) for guiding light, supporting hardware to hold members of the set of light sources with respect to each other, with respect to the target cells such as, but not limited to, brain, glia, peripheral nerve, skeletal muscle, smooth muscle, cardiac muscle, pancreatic islet cells, thymus cells, or other excitable cells, embedded in the tissue (such as, but not limited to, brain, peripheral nervous system, muscle, skin, pancreas, and heart), and perhaps held firm with respect to external structures (such as, but not limited to, skull, skeleton, muscle, or skin), and control and power electronics that monitor target cell state, provide regulated power to the light sources, communicate data, stimulation protocols, and algorithms to and from the outside world, and/or may be remotely powered by external electromagnetic fields or other kinds of wireless energy.

It should be understood by one of ordinary skill in the art that each of the variations on the component parts of the present invention are swappable with any of the other variations. Similarly, when a use of the present invention is described with respect to a particular tissue or body part, it will be understood by one of ordinary skill in the art that the invention can be used in a similar manner for other body parts and tissues. For example, if a use is described is for the “brain,” then it may similarly used in any other bodily tissue (e.g., peripheral nerve, pancreas, etc.). As another example, if it is described how to affix something to the skin, it may similarly be used in dealing with muscle and other tissues as well. The terms light source, LED, or laser are also used interchangeably, as they all have similar functionality in the context of the present invention. The light produced by the source may be visible light, infrared, spectrally complex, or any other type of light found to be suitable for the particular application.

A set of light sources may be used, because tissues are highly scattering, so that in many cases no one light source will be able to illuminate all the target cells in the entire desired target area. Each individual light source must receive electrical power, and deliver light locally to its target cells. In one embodiment, each light source is coupled to an optical fiber that projects deep into the tissue of interest to deliver light to the target cells. The electrical leads of the light source extend to the power/control circuitry, which provides timed pulses of electricity to the light source. The entire set of light sources may comprise many such optical elements, and in an illustrative implementation are arranged in an array on supporting hardware, with all the light sources in a plane, the fibers projecting perpendicularly into a tissue, and the ends terminating in various target regions where the target cells reside. In alternative implementations, the plane is a flexible substrate, so that fibers project inward from a curved surface into the tissue (e.g., if the target cells are in a tissue that is a naturally curved substrate like the brain), or there are multiple flat planes connected at their edges (e.g., forming part of a polyhedron).

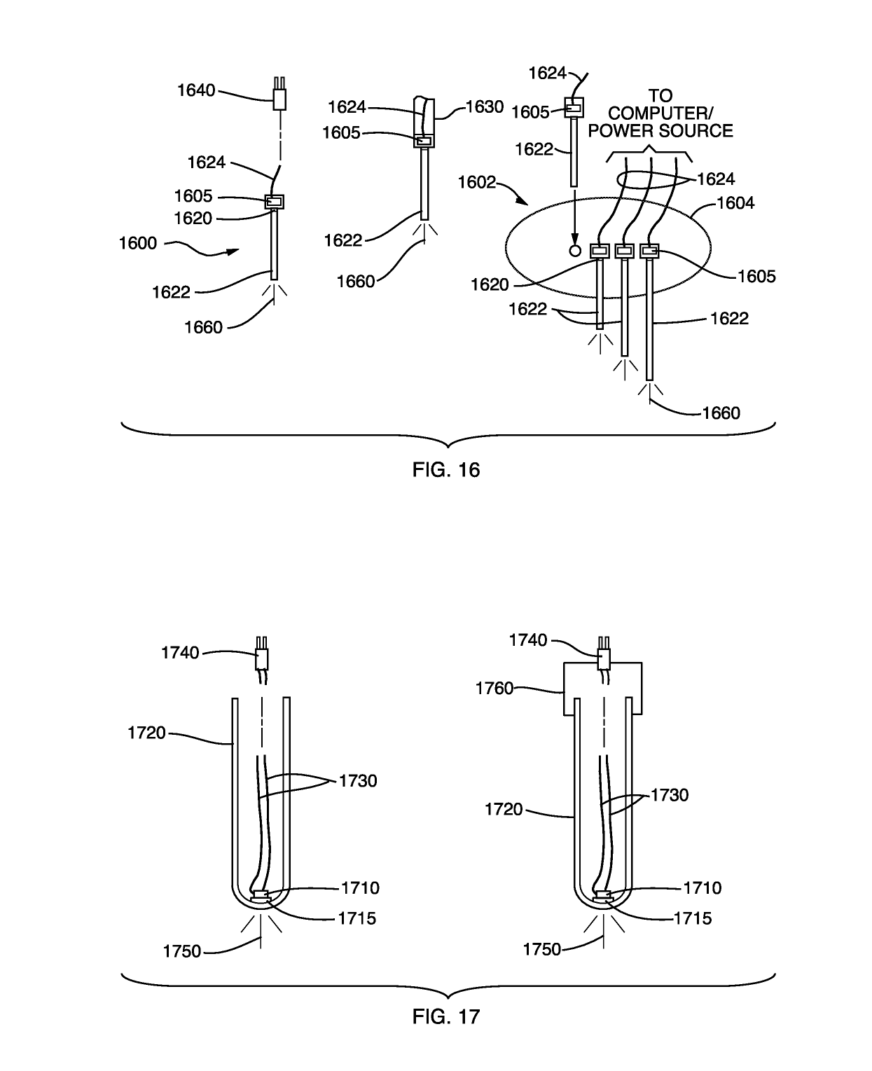

FIG. 16 is a schematic diagram depicting fiber-coupled LED element 1600 and array 1602 composed of multiple such elements, attached to plate 1604, according to one aspect of the present invention. In FIG. 16, LEDs 1605 (such as, but not limited to, yellow or blue ones) are shown glued with optical adhesive 1620 to optical fibers 1622, each with a wire 1624 (such as, but not limited to, copper) emerging in the direction opposite to the direction of a fiber 1622. The wires can run through optional cannula 1630 for protection, strain relief, and biocompatibility, and are optionally attached to electrical socket 1640 at the end to provide easy attachment and disconnection. Cannula 1630 may be made of any suitable material known in the art including, but not limited to, stainless steel, titanium, or glass. The target ends of the fibers are aimed to deliver light 1660 to specific target cells in the target tissue, and can terminate at different depths within it. For example, in the brain, the target cells might be neurons. The array holder plate may be made of any suitable material known in the art including, but not limited to, PCB board, kaptan, or steel. It may also alternatively be a hollow scaffold-type design, rather than being a solid plate.

An alternative implementation of the set of light sources is to place the LED at the tip of a hypodermic or cannula, attached to the walls of the cannula with optical adhesive and optionally coated by a biocompatible coating. FIG. 17 is a diagram depicting a hypodermic LED source, according to this aspect of the present invention. LED 1710 is attached by optical adhesive 1715 at the aperture of implantable hypodermic 1720. In an illustrative implementation, hypodermic 1720 is steel, but it may be any suitable material known in the art. Wires 1730 attached to LED 1710 snake up tube 1720, which is optionally attached to connector 1740. This has the advantage of delivering light 1750 directly to the area of interest, while minimizing fiber-coupling losses. Optional housing 1760 may also be used. The need to place a large LED at depth may require more room for the implantation, as opposed to a very small fiber, but as LEDs become smaller and smaller, this difference will become moot. Calculations indicate that tissue heating due to local light generation will be negligible for almost all clinically relevant applications of the LED. The LEDs and hypodermics can optionally be assembled into an array, as shown in FIG. 16.

Another illustrative implementation has a bare LED, potted in a biocompatible coating, with wires leading out of the coating. Yet another illustrative implementation has the LED on a peripheral nerve cuff (e.g., as used in nerve cuff electrodes), which brings the LED in close apposition to a nerve that is desired to be stimulated. This enables stimulation of peripheral nerves, e.g. for sensory replacement, controlling motor outputs, or silencing pain neurons.

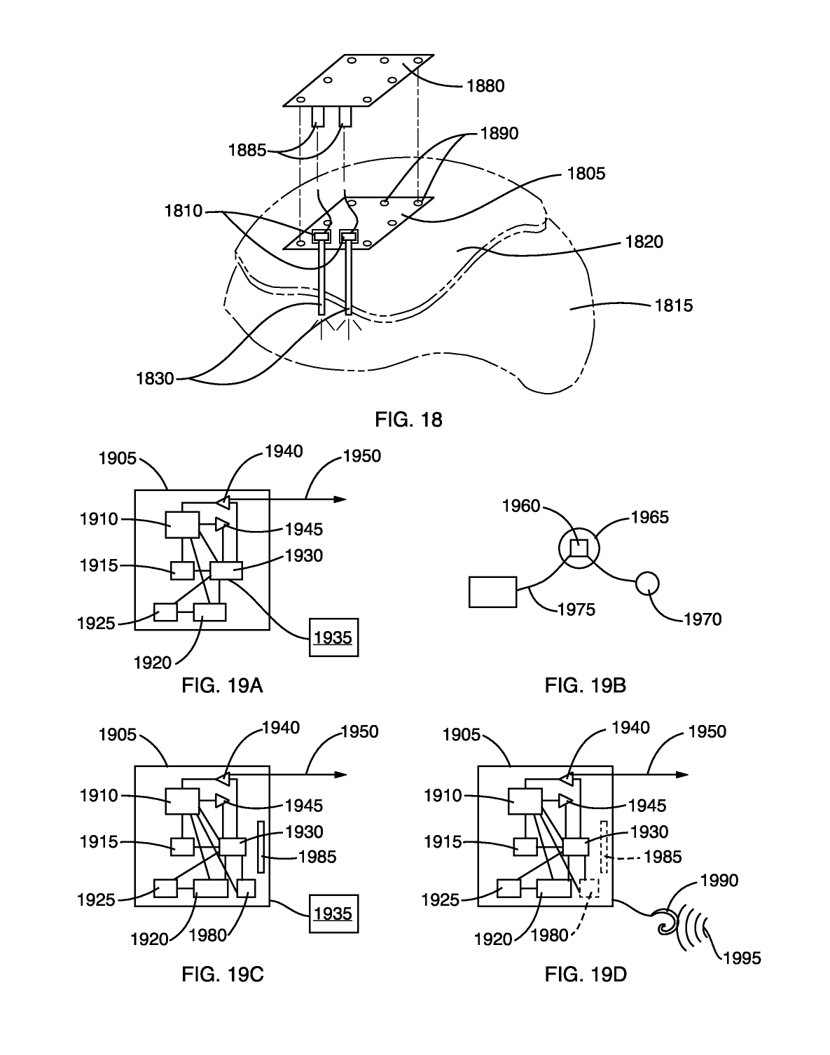

FIG. 18 depicts an embodiment of a plate for holding the circuitry and LEDs, according to one aspect of the present invention. Plane 1805 containing all the light sources can correspond to a physical plate made of printed circuit board materials, including, but not limited to, kapton, polyimide, titanium, and stainless steel, that holds LEDs 1810 firmly oriented, via adhesive or mechanical fitting into holes, towards the targets on brain 1815 and skull 1820. Plate 1805 may be conformal or may alternatively be of scaffold-type design. LEDs 1810 may be connected to optical fibers 1830, as in FIG. 16, or within hypodermic tubes, as in FIG. 17. The cannulas, plates, sockets, etc. make up the supporting hardware, which is designed to connect to electronics board 1880 (and FIG. 19) via sockets 1885 and holes 1890 for screws, dental acrylic, or other means known in the art for docking board 1805 with the upper layers of the device. The embodiment of FIG. 18 is suitable for use with many different types of implementations, including wearable, implanted, wirelessly-controlled, or remotely-powered implementations. The device of FIG. 18 is capable of being implanted under the skull, within the brain, or in within one or more parts of the body.

In illustrative implementations, the light source has one or more wires emerging from the supporting hardware. These wires lead to the control and power electronics. The wires need not be physical strands; instead, multiple circuit boards can directly dock with one another. The control and power electronics contain all of the elements needed to power the light sources when light is desired, to perform any necessary computations, to communicate with the outside world to obtain light pulse programs or to upload data, to store data locally, to acquire power from remote sources, or to detect local phenomena in the brain circuit (including, but not limited to, spikes or field potentials detected on an electrode) in order to react appropriately and deliver light of the appropriate wavelength, power, timecourse, etc. For example, a particularly appealing way to modulate LED power with a simple circuit is to pulse width modulate (PWM) the LED. A particularly simple wireless method is to simply attach an LED to an inductor, which is then remotely powerable.

Various embodiments of these circuits are battery-powered, wearable, fully implantable, wirelessly-operated, and/or remotely-powered, so different versions of the electronics may be advantageously employed to drive the LEDs. FIGS. 19A-D are diagrams depicting four alternative electronics boards for operating the fiber array, according to this aspect of the present invention. Wearable (FIG. 19A) implementations contain all the computational and power capacity onboard, as do implantable (FIG. 19B) versions. As shown in FIG. 19A, board 1905 supports microcontroller 1910, preferably with D/A converters (such as, for example, but not limited to, a PIC microcontroller), RAM 1915, flash memory 1920 to store the pulse program, and USB 1925 for uploading programs and downloading data and/or logs. On-board DC power source 1930 is supported by battery 1935, which is an Li ion battery in an illustrative implementation but could also be any other suitable battery or other power source known in the art including, but not limited to, an ultra capacitor or even a wall connection. Board 1905 also supports amplifiers 1940, 1945 or other circuits to drive the LEDs and electrodes or other neural sensors 1950, which provide information that can permit microcontroller 1910 to trigger light pulses in a dynamic way. As shown in FIG. 19B, LED 1960 is embedded in biocompatible coating 1965, powered by battery 1970, and is connected 1975 to a board that is similar to, or the same as board 1905 from FIG. 19A.

Wirelessly-operated devices, such as the one shown in FIG. 19C, are implemented like the wearable and implantable devices of FIGS. 19A and 19B, but they also comprise transceiver 1980 and antenna 1985 in order to receive and transmit information via RF. While RF transceiving is described, it will be clear to one of ordinary skill in the art that any kind of wireless communication may be advantageously employed in the present invention, including, but not limited to, ultrasound and optical. Remotely-powered devices, such as the one shown in FIG. 19D, may include antenna 1990, specialized for the capture of magnetic or RF energy 1995, such as, but not limited to an inductor, power RF coil, or RFID chip. They can also be wireless, like the embodiment of FIG. 19C by incorporating transceiver 1980 and antenna 1985. Depending on the disorder being treated, the duration of the treatment, and the risks associated with various kinds of implant, various subsets or combinations of these specifications may be found to be desirable for a particular individual patient.

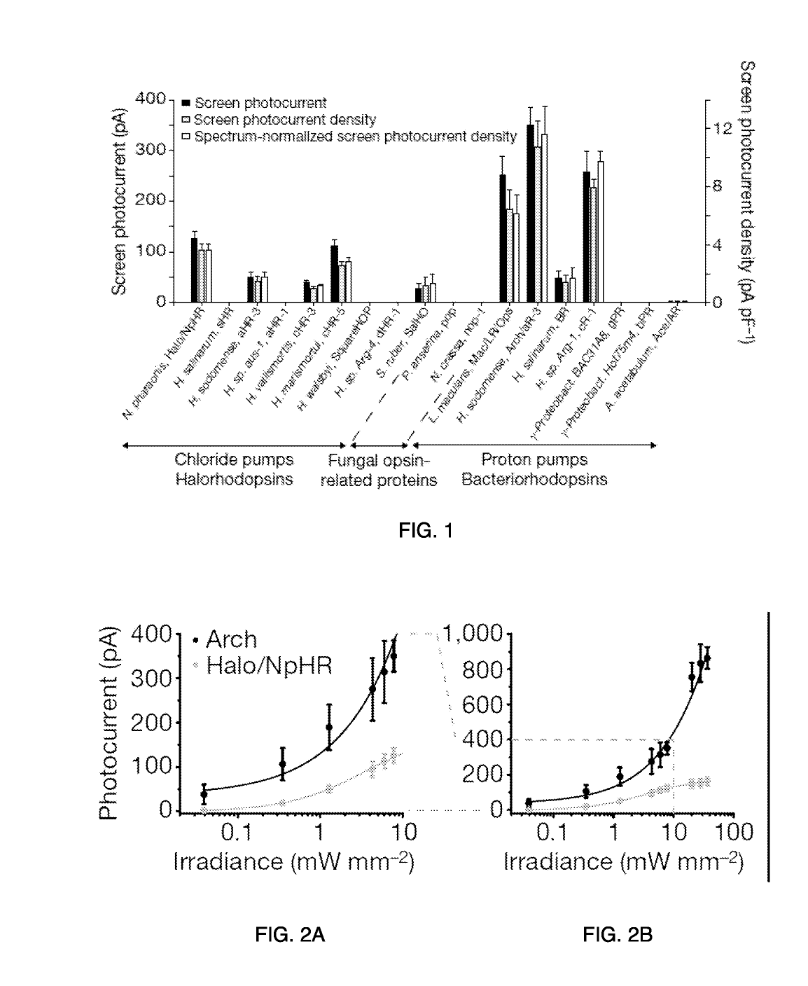

In an example implementation, specific to the brain and skull, materials used include unjacketed optical fiber—100 μm, 200 μm, or 500 μm UV-VIS transmitting (FIG. 1), ultra-thin wall stainless steel hypodermic tubing, ultrabright blue LEDs (e.g., EZ1000 for coupling to fibers (FIG. 16) or EZ290 used for coupling to fibers or being implanted directly in brain in hypodermic (FIG. 17)), ultrabright yellow LEDs (Luxeon III, Luxeon Rebel, used for coupling to fibers (FIG. 16), Lumileds P4—implanted directly in brain in hypodermic (FIG. 17)), and optical adhesive. Tools used may include UV curer, Dremel, water jet cutter, laser cutter, excimer laser, and 3-D printer. The fiber array is made up of two components—supporting hardware (FIG. 18), and a collection of modular light guides (FIGS. 16 and 17). For small structures, 100 μm and 200 μm optical fiber light guides may be used (FIG. 16), whereas for larger structures, hypodermic light guides may be used (FIG. 17).

For assembly of this example implementation, the lowest layer of the supporting hardware is cut on an excimer laser, with holes for screws to attach the supporting hardware to the skull. The second layer of the supporting hardware screws or pops onto the first, and is a printed circuit board, containing a wireless transceiver, an embedded antenna, a programmable IC, and circuitry to drive current through the LEDs in the light guides (FIG. 18). It interfaces with each light guide through a custom plug. Each light guide has a clearance hole in both layers, as well as docking holes for the housing in the first layer. The light guides are encased in a custom 3D printed housing. At the top of each light guide is a socket to interface with the electronics on the supporting hardware. At the bottom of each light guide is an opening for the optical fiber or LED to emerge. Steel cannulas are cut circumferentially with a Dremel to avoid collapsing or crimping the tubing. For the optical fiber light guides (FIG. 16), the LED sits inside of the housing and is coupled directly to the fiber with optical adhesive, generating light that is sent down the optical fiber, which is implanted directly in the brain. For the hypodermic light guide (FIG. 17), a thin walled stainless steel tube is wired with a 300 μm wide LED at the base, which shines light directly into the brain. Any exposed wire is insulated with biocompatible coating. This particular implementation can shine light about 0.5-1 mm away from a typical fiber (diameter 0.2-0.5 mm).

These fiber arrays may be implemented using individual lasers or LEDs, but arrays of vertical cavity surface emitting lasers (VCSELs) or other optical sources can be used as well. It is further envisioned that if, in the future, xenon bulbs, halogen lamps, incandescent bulbs, or other light sources become miniaturized enough to fit, they may also be advantageously used in the prosthetics of the present invention (e.g., with filters on the bulbs), although current embodiments of these devices are not as viable as LEDs and lasers due to their wasted energy, expense, danger, and limited life.

An optional enhancement is the use of a dichroic (or beamsplitter, or other equivalent optical part) attached to a fiber in a way so that it couples two different light sources (e.g., a blue LED and a yellow LED, or a blue laser and a yellow laser) into the fiber, so that the target cells at the end of the fiber can be activated and deactivated by two different colors of light (see, e.g., X. Han and E. S. Boyden, “Multiple-color optical activation, silencing, and desynchronization of neural activity, with single-spike temporal resolution”, PLoS ONE 2, e299 (2007)). Also suitable is a series of cascaded dichroics, capable of coupling more than two colors of light into the same fiber. Another optional enhancement is a steerable element (such as, but not limited to, a galvanometer, an acousto-optic deflector, a MEMS mirror, or other steering device), on one or both ends of the fiber, in order to direct light in a controlled way, enabling locally selective targeting of the light to specific areas of the tissue, preferably with as few moving parts as possible.

In illustrative implementations, this invention may use light to excite and inhibit electrically excitable cells. Alternatively, in some implementations, this invention may be advantageously employed to deliver light to other realms, such as to drive the production of cAMP in deep tissue [Schröder-Lang S, Schwärzel M, Seifert R, Strünker T, Kateriya S, Looser J, Watanabe M, Kaupp U B, Hegemann P, Nagel G. “Fast manipulation of cellular cAMP level by light in vivo”, Nature methods (2006)], to simulate the action of a G-protein coupled receptor acting drug [J. M. Kim, J. Hwa, P. Garriga et al., “Light-driven activation of beta 2-adrenergic receptor signaling by a chimeric rhodopsin containing the beta 2-adrenergic receptor cytoplasmic loops”, Biochemistry 44 (7), 2284 (2005)], or to change the pH of a cell [G. Nagel, D. Ollig, M. Fuhrmann et al., “Channelrhodopsin-1: a light-gated proton channel in green algae”, Science 296 (5577), 2395 (2002)]. There are many therapeutic reasons to desire these abilities.

### Wireless Control:

In illustrative implementations, a supercapacitor-based electronic device delivers high currents to an array of implantable light sources or electrodes. The device receives wireless power from an external transmit coil and receives control signals from either an onboard computer or external wireless data telemetry.

The following is a description of an illustrative prototype of this invention.

In this prototype, under continuous optical stimulation, an implant can deliver >2W of power, sufficient to drive two 700 um×700 um LEDs at 100% duty cycle at programmable frequency indefinitely (e.g., ˜500 mW/mm̂2 to cortical targets), while recording from as many as 4 electrodes simultaneously. This is a very high level of power; for surface LEDs atop cortex, dozens can be run indefinitely. Intermittent LED input power can be increased to 5W for one second, enough to drive 5 LEDs at 100% duty cycle or 10 LEDs at 50% duty cycle. Intermittent high power delivery above the wirelessly supplied 2W is achieved using an onboard supercapacitor energy storage cell with >4 Joules of reserve energy, thus a scaling law for burst mode power of 2 W+4W*seconds/duty ratio. This prototype supports a maximum of 2 driven LEDs at any time, though additional driver channels are easily added with a nominal increase in weight.

In this prototype, wireless power is delivered at low magnetic field (400 A/m) and low frequency (120 kHz) to reduce the possible side effects of magnetic field exposure. Power transfer is maintained at high efficiency (10%) by utilizing a synchronized bridge driving circuit and precision frequency tuning. The receiver is also optimized for power efficiency over a wide range of the animal's body orientation by utilizing an axial resonant LC receiver coil with high permeability core and under-cage transmit coils. The power receiver circuit is optimized to produce a maximum of 2W of received power, achieved at a height of approximately 2 cm above the cage floor.

In this prototype, the modular design of the implant includes four distinct, removable subsystems: (i) a skull-fixed LED array with thermal sink, optional fiber light guides and optional 4-channel electrode pre-amplifier with 16-pin universal connector, (ii) an implant motherboard with LED drivers, power management stages and ultra-low power 16 MHz integrated microprocessor (Texas Instruments® MSP430), (iii) 2.4 GHz data telemetry link (TI®/Chipcon® CC2400), and (iv) wireless power rectifier. When not in use, components (ii-iv) can be removed from the head and subsystems can be upgraded, repaired or interchanged with other implanted LED arrays without significant disturbance to the animal.

The implant motherboard comprises an array of bare LED dies embedded in a thermally dissipative ˜1 gram copper block, which is affixed to the skull over a cranial window using 3 standard skulls crews and anchoring dental cement. A miniature printed circuit board (PCB) features wire-bonded traces from the LED control terminals to a 10-16 pin connector accepting the implant motherboard stack.

Signal conditioning electronics are embedded in the implant PCB, composed of four 10× gain common-referenced differential preamplifiers, band-pass filter and 10-100× gain (hardware adjustable) second stage amplifier. Thus, the motherboard and telemetry stack is capable of simultaneously record from 4 dc-coupled electrode channels at up to 25 k samples per second (ksps) and 10 bit resolution, low-pass filtered at 10 kHz.

Advantageously, as many as 83 separate implant-to-computer links can be simultaneously opened, by taking advantage of a Gaussian frequency-shift keying (GFSK) encoding scheme in the onboard radio and automatic frequency hopping algorithms in the ISM (Industrial, Scientific, Medical) 2.4-2.4835 GHz band. A flexible software layer on the radio allows for addressing of 2¹⁶ distinct implants, making this paradigm suitable for institution-scale high-throughput testing. Wireless communication with an implanted device can be added to any PC using a small USB dongle, each dongle supporting one full-speed implant link. Rapid modification to stimulation, recording and communication protocols is possible due to a flexible and open source C/C++ based software architecture running on the implant and PC-side systems. Real-time remote triggering of stimulation and recording systems is supported, allowing for complex closed-loop behavioral tasks and high-throughput screening of large animal cohorts.

In this prototype, power delivery to the headborne device is achieved via precisely tuned resonant inductive coupling between matched LC networks. On the receiver side, AC signal is rectified using a full-wave passive rectifier and supercapacitor energy storage element, serving to both filter AC ripple and provide reserve energy storage. This supercapacitor element improves device reliability under varying coil-to-coil mismatch angle as the animal moves around the cage, and also allows for reserve energy to be used in high-powered short duration pulsing of the implanted LEDs. Passive Zener diode shunt and active pulse width modulated (PWM) rectifier load regulation are used to maintain safe voltage range on the supercapacitor.

Following this first stage of AC/DC conversion, a switched mode buck/boost converter (Texas Instruments® TPS61202) provides low ripple conversion from the 5.5V nominal supercapacitor voltage to a regulated 3.3V digital supply (hardware adjustable, 1.8-3.6V), serving the onboard microcontroller (MCU) and both analog and digital supplies of the radio telemetry chipsets (CC2400/2500). An additional low-dropout 1.8V linear regulator, running off the 3.3V supply, provides analog power to the radio using a chip-scale packaged device (Analog Devices® ADP121). This secondary voltage supply provides extreme flexibility in future radio chipset modifications or additional peripheral circuitry.

A second buck/boost converter is utilized as a parallel LED driver circuit to power the implanted LEDs. This circuit runs directly off of the supercapacitor, with capability to operate at voltages as low as 0.3V, making it ideal for potentially variable input powers and output loading.

In this prototype, a 6″ diameter under-cage transmit coil with series capacitive elements and 3 mm diameter, 15 mm long axially wound coil with ferrite core and parallel capacitive element on the head of the animal delivers 2W of output power to the headborne device. These power levels are achieved in a low strength magnetic field of 400 A/m oscillating at 120 kHz.

Rectification of the coupled AC signal on the headborne device is performed with a full-wave passive rectifier using low forward voltage Schottky diodes and a supercapacitor filtering element of 22-150 mF.

The choice of energy storage method here is dominated by the need for rapid charging and discharging capability. Instantaneous LED currents in this prototype are on the order of 400 mA or more. A typical stimulation waveform for treatment of a Parkinson's Disease model may require 5 ms pulse width, 130 Hz stimulation, equating to a duty cycle of approximately 70%. High power density devices may be be used rather than a strictly high energy density solution, mitigating temporary bursts of stimulation over the averaged power capability of the system.

In this prototype, a supercapacitor—rather than lithium ion rechargeable cells—is used, for the following reasons: Lithium Ion rechargeable cells are an attractive option at certain scales given their substantial energy density of ˜620 J/gram, typical peak discharge rates of approximately 5 C. and pulsed rates as high as 25 C. However, several limitations to Li-Ion cells can make them disadvantageous in this design. Generally, Li-Ion batteries must be carefully regulated to prevent deep discharge below 2.0V or overcharge above 4.2V. Moreover, higher power density batteries and smaller batteries in general have a higher volume fraction dedicated to non-storage contributing elements like current substrates, thus limiting their practical energy storage. 400 mA discharge currents which may be used in this design imply a 80 mA-hour cell to maintain safe discharge rates of 5 C., while only a few Joules of energy storage can sustain surge currents experienced in photostimulation of neural networks in vivo. A one second pulsed sequence on an array of 5 LEDs operating at nominal 400 mA, 3.6V load uses 7.2 J of energy, less than 5% of the energy supplied by an 80 mA-hour batter maintained between 4.2 and 2.0V. Thus, a Li-Ion cell is highly stressed in power density yet underutilized in energy density.

By contrast, supercapacitors can achieve on the order of 10 J/gram energy density, but can sustain discharge currents of 1000C, implying that a one gramideal supercapacitor satisfies the demands of this system, while serving the additional purpose of filtering rectifier output. In practice, packaging dominates the weight and volume of these small supercapacitors.

In this prototype, a supercapacitor is used for energy storage. A prototype thin-film solution was chosen due to its low ESR (equivalent series resistance) and 5.5V limit (CAP-XX®, 300 mF, 5.5V, ESR 70 mOhm).

The ESR limit tends to be the limiting factor in terms of the maximum usable output current of most supercapacitors at the small scale (a few grams), particularly at the 120 kHz frequency operation of this prototype.

In this prototype, two DC/DC converter subsystems are present: one system (Texas Instruments® TPS61202) is dedicated to providing efficiently regulated 1.8-3.6V digital supply for the onboard MCU and sensitive RF chipset, and another (either the Texas Instruments® TPS61150 or another TPS61202) for high power up/down-conversion to drive the implanted LED array.

Both of these systems utilize off-chip inductors of 2-10 uH and input/output filtering capacitors of approximately 10 uF. Shielded inductors were chosen in both instances to minimize EMI to the RF and recording systems.

A parallel, high-side switched multiplexing topology is employed, using MCU-addressed BJTs to select which LEDs were to be driven at any time.

Advantageously, a voltage-controlled LED driver allows for substantially great output currents to be achieved. For example, with a 5.0V nominal supercapacitor supply, efficiencies of 90% and maximal output currents of 1.8A are achievable at a fixed 3.5-3.9V LED forward voltage. This rating equates to 4.5 LEDs driven at 400 mA, or 7W of output power.

Voltage-mode control does, however, result in exponential output power variation with LED forward voltage. Moreover, LED output power varies significantly with junction temperature, thus feedback is desirable in order to tightly regulate the output power of the implanted array; support for temperature sensing has therefore been built into the device. An LED under continuous operation may shift its forward voltage down by as much as 400 mV from ambient operating temperature. This 400 mV shift correlates to a change of perhaps 200 mA or >70% reduction in forward current. Thus, it is desirable to consider a nominal operating temperature when designing the LED driver circuit—in this prototype, a nominal 4V is chosen for drive voltage.

For the overall power management system of this prototype, an optimal value for energy storage exists such that the device is capable of performing a cold start—that is, the device may be placed in the presence of a 400 A/m drive field with all converter stages powered on, and sufficient storage capacitance exists to supply the initial startup energy for these buck/boost converters—yet does not have unnecessary amounts of reserve power (recall, the design specification was intended to provide 2 J of bursting energy). An analytical solution based on the load currents of a typical LED, inductor and input and output capacitor sizing would suggest a nominal supercapacitor of just a few tens of millifarads in addition to the amount of reserve power (in the 2 J case, roughly 66 mF), but bench testing suggests 2-3× this amount of energy is desirable, largely due to the buck converter's input surge current. Thus, a 300 mF supercapacitor was used for this prototype. If a smaller capacitor is necessary, the system may be powered initially off of the 1.8V linear regulator. Once supercapacitor voltage reaches full charge, the buck/boost stages can then be enabled without substantial capacitor voltage droop.

In this prototype, a headborne device features a 1 Mbps 2.4-2.4835 GHz ISM band wireless transceiver based upon the TIO/Chipcon® CC2400 integrated chipset with off-chip oscillator and differential to single-ended balun with chip antenna. An S-pin header with 3-wire SPI interface to the MCU, 2 auxiliary I/O pins, 1.8, 3.3V and ground terminals allows for independent re-design of the wireless transceiver.

The MSP430F2132 MCU runs a simple three state interrupt-based Finite-State Machine to minimize idle transceiver on-time. Transition delay between the three states, Transmit, Receive and Idle is adjustable in software in this implementation. Alternately, a channel-adaptive delay may be programmed onto the MCU to further improve performance.

In this prototype, an onboard integrated 16-bit, 16 MHz microcontroller (TI MSP430F2132) with 8-channel, 200 ksps 10-bit analog-to-digital converter (ADC), 512 byte RAM and 264 kB flash memory supervises on-the-head computation. This chipset is used to sample analog signal from the 4-channel neural recording amplifiers and optional temperature and supercapacitor voltage sensors, enable data transmission to and from the wireless telemetry, and handle pulse width modulation (PWM) of the LED drivers to in turn modulate neural network activity in vivo.

In this prototype, the overall control architecture is as follows:

Initialization: Upon startup, the MCU transmits 44 data frames of 24 bits each to initialize the radio chipset. By default this initialization is at 2.4000 GHz transmit frequency, Gaussian Pulse Shift Key (GPSK) modulation, 1 MHz channel width, and 1 Mbps data rate via unbuffered transmit mode. A CRC is, however, calculated.

Channel ID: The remote device transmits its unique channel ID periodically at default frequency of 2.4000 GHz, awaiting instruction from the PC-side controller, implemented as a simple Hyperterminal interface to a USB-connected 2.4 GHz transceiver.

Control Signals: Control signals from the PC-side controller trigger state change in the remote device—4 Neuromodulation parameters, namely LED address, pulse frequency, pulse width and on/off toggle—and 3 Recording parameters, namely electrode channel select, sample rate and on/off toggle are all transmitted to the remote device with unique device ID. In addition, optional LED temperature and supercapacitor voltage monitoring can be toggled, to have the remote device transmit real-time state variables to the PC. These 9 control variables may be updated at any time by text entry into the Hyperterminal window. Finally, a frequency hopping routine can be initiated to find the nearest clear channel in the ISM band.

Neuromodulation Programming: On the remote device, these pulse frequency and pulse width parameters are stored as 16-bit comparator trigger values. An up/down waveform generator operating at 1.2 MHz is toggled on and off by the on/off toggle control signal, such that the comparator output triggers the particularly addressed LED on/off with a square wave. This allows for independent control of two LED waveforms simultaneously, and 8-LED addressing using a direct port-to-port mapping of pins. Alternately, an addressing scheme may be implemented using a binary addressing scheme.

Pulse Frequency Programming: Frequency is transmitted to the device as a decimal number (e.g. ‘FR=130’=130 Hz). On the device, decimal to hex conversion is performed, and the upper trigger register (TACCR0) is set as TACCR0=Clock frequency/2*Pulse frequency.

Pulse Width Programming: Pulse width parameter is transmitted as a decimal number in milliseconds (e.g. ‘PW=5’=5 ms). On device this is converted to hex, and stored in the Toggle/Set register (TACCR1 for LED channel 1, TACCR2 for LED channel 2), as TACCR1/2=PW*Clock frequency*500.

Recording Programming:—On the remote device, the electrode channel select and sample rate are programmed into a direct memory access (DMA) controller which automates the sampling of data from the 10-bit ADC to memory or flash. By default, data is written temporarily to memory and then output unbuffered to the radio for streaming recording.

Supercapacitor Overvoltage Control:—Active PWM control of supercapacitor voltage is employed. The MCU's comparator's interrupt flag is enabled to alert the system in the event that voltage on the supercapacitor exceeds safe 5.5V threshold. In this instance, the MCU open circuits the rectified in a back-off-and-wait manner for a programmed period of time.

Channel Frequency Hopping:—In modes of operation where either multiple animals are under simultaneous control, an optional channel hopping protocol can be initiated between PC-side and remote device to find the nearest clear channel.

In this prototype, the headborne device can be easily disconnected from an implanted optics array. This array comprises a set of bare die LEDs affixed to a small copper block, which serves as thermal sink to draw lost energy away from the brain. LEDs are wire bonded to a small PCB also connected to the copper block, which serves as an anchor point for a 10-16 pin Samtec® connector allowing easy disconnect of the headborne electronics for repair or replacement, to ease animal housing needs, etc.

In the event that deep brain structures are targeted for optogenetic control, small fiber waveguides are affixed directly to the surface of the LED die using optics glue, and the remaining die surface coated with reflective epoxy. An additional reflector is placed behind the die for secondary redirection. Significant loss of energy results from this arrangement due to the uncollimated nature of LED light. Alternately, lasers may be used as light sources to reduce these energy losses. Still, with the LEDs in this prototype, output power at the tip of the fiber is approximately 5-10 mW/mm̂2, sufficient for local activation of ChR2 targeted neurons.

In this prototype, an integrated 8-channel 10-bit ADC affords the ability to monitor critical device parameters remotely. This feature is useful for diagnostic purposes in the event of aberrant behavior on the part of the animal, and also adds an element of safety to the device by allowing remote shutdown in the event of supercapacitor overvoltage, over-current (indicating a short circuit), or over temperature events. Many of these features are also hard-wired into the existing circuitry, e.g. over-temperature shutdown in the TPS61202 and over-current limiters. Specific designs are listed below.

In this prototype, rectifier current is monitored. This monitoring is useful not only for diagnostic purposes, but also in the event that alternative power transfer techniques are employed. Magnetostrictive/Electroactive (ME) sandwich materials for power conversion can be employed. In such designs, resonant frequency is tightly coupled to the temperature of the core material. As such, a constant monitor of rectifier current can be used to optimize drive coil tuning frequency over a wide range of implant temperatures.

In this prototype, a high-side current shunt monitor is used to measure small voltage drops across a precision sense resistor placed in series with the supercapacitor load. The shunt monitor (Texas Instruments® INA193) amplifies the voltage drop across a 0.3 Ohm sense resistor by a factor of 20-100V/V and outputs this voltage to one of the eight ADC channels with operating range of Vdd. This implies a maximal sensed current of 500 mA for 3.3V supply, sufficient for a 2.5W rectifier with 5.0V nominal capacitor voltage. Power loss due to the sense resistor is Î2*Rsense=75 mW. Reduction of sense resistor below 0.3 Ohms linearly reduces power loss, though values much below this may require careful layout to achieve accurate readings. Alternately, a current sense FET may be used (which may be advantageous in higher power settings or where dissipation is critical).

As already described, supercapacitor peak voltages are generally limited to only a few volts before breakdown occurs. Catastrophic breakdown is very easily achievable with the power figures developed in this system.

Thus, in this prototype, a first order protection circuit implements a current shunt. The 5.5V peak allowed by the supercapacitors is convenient for use of a Zener diode at 5.1 or 5.6V, though care must be taken to ensure that the diode is rated for peak power dissipation of several watts. An active monitoring loop is also implemented, in which an N-channel depletion mode transistor (FET) is placed in the conduction path from rectifier to supercapacitor. The depletion mode FET normal conducts when gate voltage is zero, allowing the supercapacitor to charge even though the digital control loop is not yet powered on. Once supercapacitor voltage is high enough for the digital supply to turn on and initialize the microcontroller, the microcontroller begins actively sampling supercapacitor voltage. If voltage sampled is above the threshold voltage (5.5V) for safe operation, a positive gate voltage is applied to turn off the control FET for a fixed duration, defined in a “back off and wait” paradigm.

A recording amplifier with 4 input channels, one serving as common mode ground, is built into the implant headstage motherboard. The first stage amplifier is a unity to 20× gain, capacitively coupled single rail amplifier based upon the Texas Instruments® TLC2264 quad operational amplifier. All four channels including the reference channel are buffered in this manner. With the first channel serving as common ground, the outputs of the first stage are fed into a 10-1000× programmable gain instrumentation amplifier (INA333) with first order low pass filter set to approximately 10 kHz. The input impedance of the system is approximately 10¹² Ohms. Adjustment of the second stage gain is done in hardware, by replacing the gain setting resistor on each of the INA333's. Advantageously, these amplifiers are easily swappable given the modular design, such that any preamplifier producing a controllable gain output voltage can be utilized.

Thermal dissipation of electronic components on the head is a significant concern. In this prototype, numerous design principles have been employed to limit the amount of unwanted heat generation. Where possible, dissipative elements (sense resistors, diodes, pass transistors) have been chosen to reduce conduction loss. The microcontroller and radio elements can operate in a low power mode, with rapid startup times on the radio oscillator core and the MCU.

In this prototype, a modified USB-to-serial JTAG programmer serves as a base for a wireless base station for communication with the remote device.

The USB programmer/wireless base station has a 16-pin Samtec® header for mounting an implant motherboard and radio PCB. With a simple re-flashing of memory, the implant becomes a 1 Mbps tether. The original TI MSP430-UIF programmer (red PCB) has been modified to allow for full speed communication with the USB port, as default units are limited to 9600 baud backchannel communication.

In this prototype, all PC-side interface software is maintained in firmware in the tethered device; Windows Hyperterminal software is used. The USB device reports itself as a serial interface with a dedicated COM port. The user connects the USB device, opens a new Hyperterminal session using the COM port associated with the USB port on their PC, and the tethered device automatically initializes with a splash screen listing the command-line options for communication with tethered devices. All keystrokes are parroted back to the user, such that the link appears to be native software.

In this prototype, a USB-connected wireless tether unit is employed. A modified JTAG interface microcontroller programmer is used to open a serial COM port between a docked electronics stack and a host PC. The microcontroller handles all network protocols to communicate with up to 83 devices simultaneously on ISM 2.4 GHz band in 1 MHz channel spacing, and communicates with the PC using standard UART protocol. A 16-bit identifier system allows up to 2̂16 different devices to be controlled using intermittent polling.

To demonstrate the high power capacity of the headborne wireless optical stimulator, a Parkinson's Disease (PD) mouse model was wirelessly treated using a simple, autonomous version of this invention. A headborne system was pre-programmed to generate a 130 Hz, 5 ms pulse width waveform previously reported to successfully halt Parkinson's like behavior in the PD mouse model. The device was targeted toward right hemisphere M2 motor cortex, some of whose afferent axons projection to the subthalamic nucleus, a target known to be effective in deconstruction of Parkinsonian essential tremor. The PD behavioral phenotype is modeled as a rightward tendency to rotate on the part of the animal. When stimulated with the wireless system, the animal shows a halting of rotational behavior. This behavior has recently been demonstrated in fiberoptically tethered animals, however, the power requirements of stimulation, which may be roughly 1W input power to the LED, previously made wireless operation inaccessible.

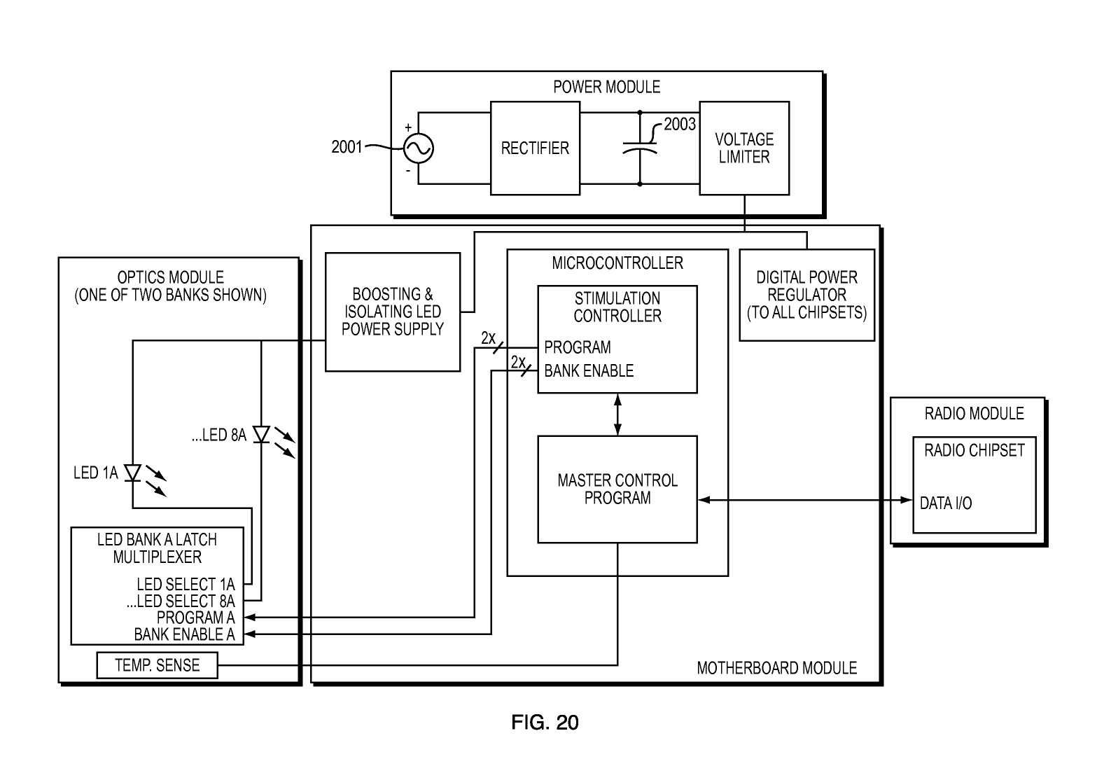

FIG. 20 is a schematic side view of a wirelessly powered and controlled headborne electronics architecture with implanted light sources, in a prototype of this invention. The optics module, housing light sources (LEDs), temperature sensing and LED multiplexers are affixed to the skull, while other modules are detachable. A motherboard module, with embedded microcontroller, LED driver and power management circuitry, delivers pulse waveforms to implanted light sources via 6-pin connector. Additional pins allow for in-the-field reprogramming of electronics stack with USB computer adapter. A power module includes a power antenna 2001 and a supercapacitor 2003. The power module connects to motherboard using 3-pin connector, and contains wirelessly coupled power from under-the-cage transmitter. Passive and active voltage limiting circuitry protects from potentially damaging over-voltage events. A minimal configuration without radio module allows for chronic delivery of pre-programmed waveforms for open-loop neuromodulation experiments. Finally, real-time continuous monitoring and control of up to 83 headborne devices in a given shared radio space is enabled with 1 Mbps radio module using a standard PC with USB-connected wireless dongle. Thousands of devices may be intermittently addressed using polling schemes, allowing for high throughput behavioral research.

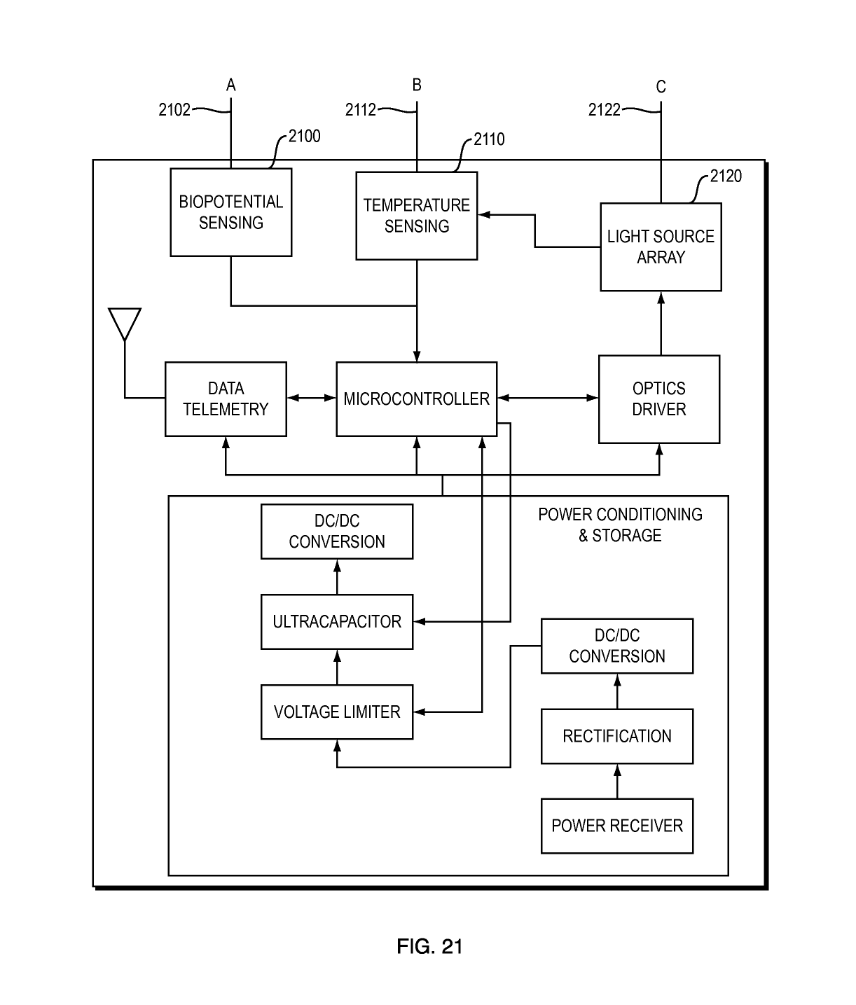

FIG. 21 is a schematic diagram of a headborne/implant device that is powered by an external power transmitter and that employs a data telemetry link. The device comprises the items within the outermost box in FIG. 21. Biopotential sensing amplifiers 2100 are linked by a wired or wireless connection 2102 to electrodes (not shown) near the headborne or implantable device for closed-loop operation of the light source array. Internal temperature sensor 2110 is linked by a wired or wireless connection 2112 to nearby tissue (not shown) for closed loop operation of a light source array safety cutoff. Light from a light source array 2120 is transmitted by a transmission means (e.g., fiber optic or lens) 2122 to nearby tissue (not shown).

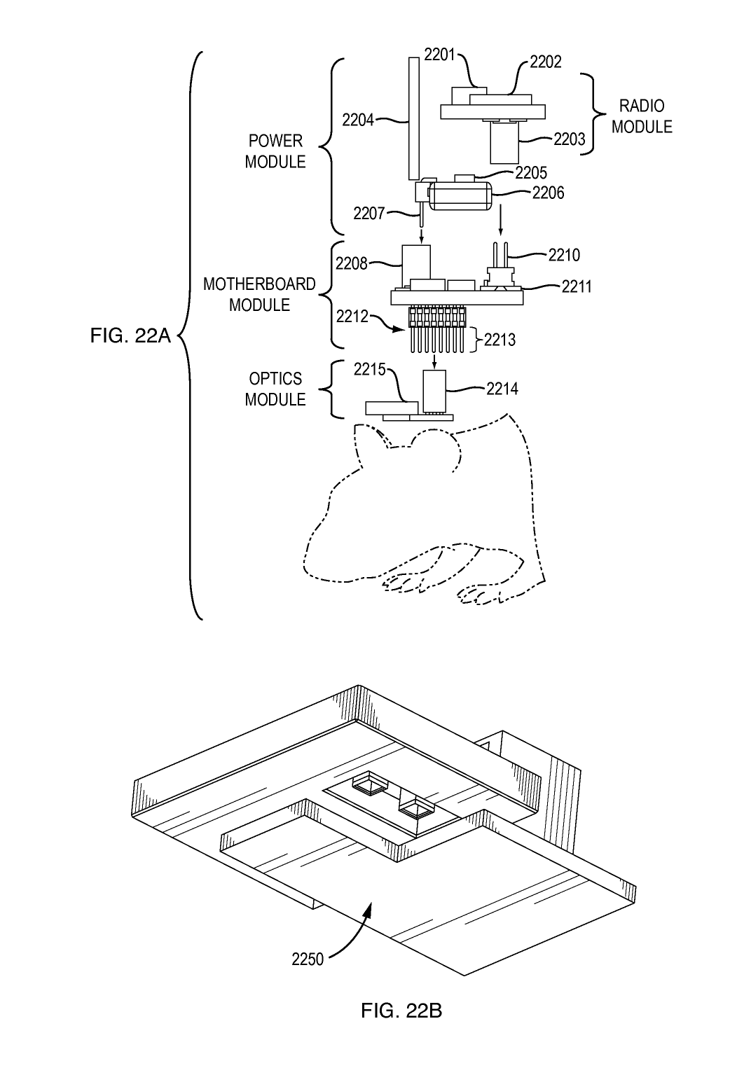

FIG. 22A is a block diagram of a headborne/implant system, in an illustrative implementation of this invention. Four total modules are represented: Power management, Optics, Motherboard and Radio modules. Wireless power is rectified and stored using onboard supercapacitor, enabling intermittent bursts of 5W of power to optics module. Up to 16 LEDs, grouped into two 8-LED banks are independently addressable using latch circuitry. Banks may be controlled separately by two different control waveforms. Radio features high-speed 1 Mbps wireless data link for realtime remote control of hundreds of headborne units. As shown in FIG. 22A, the headborne system includes a radio antenna 2201, radio transceiver chipset 2202, female radio receptacle 2203, power receiver antenna 2204, power rectifier circuitry 2205, supercapacitor 2206, male power connector 2207, motherboard power receptacle 2208, microcontroller 2209, male radio connector 2210, analog power buck/boost converter 2211, male interface connector 2212, male programming interface pins 2213, female optics interface receptacle 2214, and LED thermal sink 2215.

FIG. 22B is a perspective view of the underside 2250 of an optics module, in an illustrative implementation of this invention.

In illustrative implementations, this invention has many practical applications: Among other things, the wireless optical neural control system may be used for in vivo neuroscience research. The freedom to explore complex environments while maintaining recording and optical neuromodulation capability (which illustrative implementations of the present invention allow) is highly desirable. The ability to remotely address dozens of device simultaneously presents an opportunity to perform high-throughput screening of complex behaviors. Whereas previously researchers were required to select the animal, tether the animal singly, perform the experiment and begin anew, such research can become at least semi-automated. Additionally, closed-loop paradigms in complex environments, in which an optogenetic control event is triggered based upon either neural network activity or behavioral event, can be easily implemented without modifying the hardware or software interface already developed.

This invention may be implemented in many different ways, and is not limited to the prototype implementation described above. Here are some non-limiting examples:

An alternative embodiment of this invention includes a highly miniaturized application-specific integrated circuit (ASIC) with RF telemetry system, microcontroller, low channel count neural amplifier and light source controller on a single piece of silicon, with thin-film supercapacitor, solid state laser element and power and data antennas all packaged in a sub-millimeter scale device. Such an assembly is similar in function and system architecture, but through miniaturization using standard VLSI and thin film processing techniques art, the miniaturized device may be injected into target brain regions in the human. Independent devices can be programmed with different stimulation waveforms stored in microcontroller memory to modulate differing brain regions with spatiotemporal specificity. Closed-loop algorithms may be implemented by sensing on the integrated neural amplifier and delivering the appropriate optical modulation signal algorithmically. A single external power transmitter can safely power all devices using frequencies in the sub-MHz band. Using such a system, whole-brain modulation can be employed without the need for a bulky implant and large numbers of long-distance optical fibers.

Different types of power sources may be used in this invention. For example, the supercapacitor may have a thin-film, tantalum, or electrolytic wet cell design. The supercapacitor design may employ series stacking of matched elements. The number of supercapacitors may vary. One or more supercapacitors may be employed. Alternately, batteries may be used, although these may have disadvantages in many applications, as discussed above. Alternately, a wired power source may be employed, although this may require a tethered or immobile subject.

Light sources other than LEDs may be employed. For example, lasers may be used.

In some implementations of this invention, thermistor-based temperature monitoring circuits are employed. For example, a simple resistor divider circuit using a remotely located thermistor epoxied to the LED heat sink, in series with a bias resistor, can be placed across the input to one of the ADC channels. Capacitive filtering can be added, using the input resistance of the ADC to create a high-pass filter pole. (Such capacitive filtering can be employed for all voltage-based sensing used in the system). Also, for example, the LED drive systems can incorporate junction temperature compensation.

This invention may be implemented as apparatus for optical control of tissue of a living organism, which apparatus comprises: (a) at least one supercapacitor for energy storage; (b) one or more light sources, (c) an array of optical fibers or light guides for delivering light from the one or more light sources to the tissue; and (d) an antenna and circuitry for receiving power by wireless transmission from an external transmit coil. Furthermore: (1) the apparatus may be adapted for implant in the living organism; (2) the wireless transmission may comprise transcutaneous energy transfer; (3) the apparatus may be adapted for cranial implant and the living organism may be a mammal; (4) the apparatus may be adapted to be positioned adjacent to an exterior surface of the living organism, in a position such that the one or more light sources are partially or wholly located externally to the living organism and the array is at least partially inserted into the living organism; (5) at least some of the one or more light sources may comprise light emitting diodes; (6) the tissue may be neural tissue; (7) the apparatus may further comprise sensors for recording neural activity in the organism; (8) the apparatus may further comprise a 3-axis power receiver antenna, (9) the apparatus may further comprise a DC/DC converter for reducing voltage of wirelessly-received energy, after rectification and before delivery to the supercapacitor; (10) the supercapacitor may store energy, which energy is received wirelessly from an external source continuously or more than once every 24 hours; (11) the apparatus may further comprise a DC/DC converter for tuning RF power link open circuit voltage; (12) the apparatus may also comprise a DC/DC converter circuit for delivering an output voltage over a range of capacitor voltages, which output voltage does not vary more than 15%, or for delivering an output current over a range of capacitor voltages, which output current does not vary more than 15%; (13) at least part of the converter circuit may have either a buck/boost or charge pump topology; (14) the apparatus may further comprise a processor for adaptively controlling light output from the one or light sources, based at least in part on an algorithm that models heat transfer in the tissue; (15) the apparatus may further comprise a processor for generating control signals to shutdown light delivery if an increase in tissue temperature exceeds a specified threshold; (16) the processor may accept user input to change the specified threshold; and (17) the apparatus may further comprise one or more sensors for measuring biopotentials.

This invention may be implemented as an implant device for implantation into a living organism, which implant device comprises at least one supercapacitor for energy storage, one or more electrodes for electrical stimulation of tissue of the living organism, and an antenna and circuitry for receiving power by wireless transmission from an external transmit coil by transcutaneous energy transfer. The implant device may further comprise a processor for generating control signals to reduce power dissipation if an increase in tissue temperature exceeds a specified threshold.

### Prosthetic System:

In illustrative implementations, an optical prosthesis that permits control of neural circuits comprises a probe having a set of light sources, drive circuit connections connected to each light source, a housing surrounding the light sources and drive circuit connections, and drive circuitry for driving and controlling the probe. The drive circuit connections and drive circuitry may optionally provide for wireless communication. The light sources may be light-emitting diodes, lasers, or other suitable sources. The device may optionally include sensors for monitoring the target cells. In an illustrative implementation, the present invention may comprise a multi-dimensional array of probes, each probe having a set of light sources, drive circuit connections connected to each light source, a housing surrounding the light sources and the drive circuit connections, drive circuitry for driving and controlling the probes, and supporting hardware that holds the probes in position with respect to each other and the target cells.

In illustrative implementations, the present invention may comprise several parts: a set of light sources (such as, but not limited to, LEDs or lasers) with optional accessory hardware for guiding light, supporting hardware to hold members of the set of light sources with respect to each other, with respect to the target cells such as, but not limited to, brain, glia, peripheral nerve, skeletal muscle, smooth muscle, cardiac muscle, pancreatic islet cells, thymus cells, or other excitable cells, embedded in the tissue (such as, but not limited to, brain, peripheral nervous system, muscle, skin, pancreas, and heart), and perhaps held firm with respect to external structures (such as, but not limited to, skull, skeleton, muscle, or skin), and control and drive electronics that monitor target cell state, provide regulated power to the light sources, communicate data, stimulation protocols, and algorithms to and from the outside world, and/or may be remotely powered by external electromagnetic fields or other kinds of wireless energy. A set of light sources is needed because, since tissues are highly scattering, in many cases no one light source will be able to illuminate all the target cells in the entire desired target area. Each individual light source must receive electrical power and deliver light locally to its target cells.

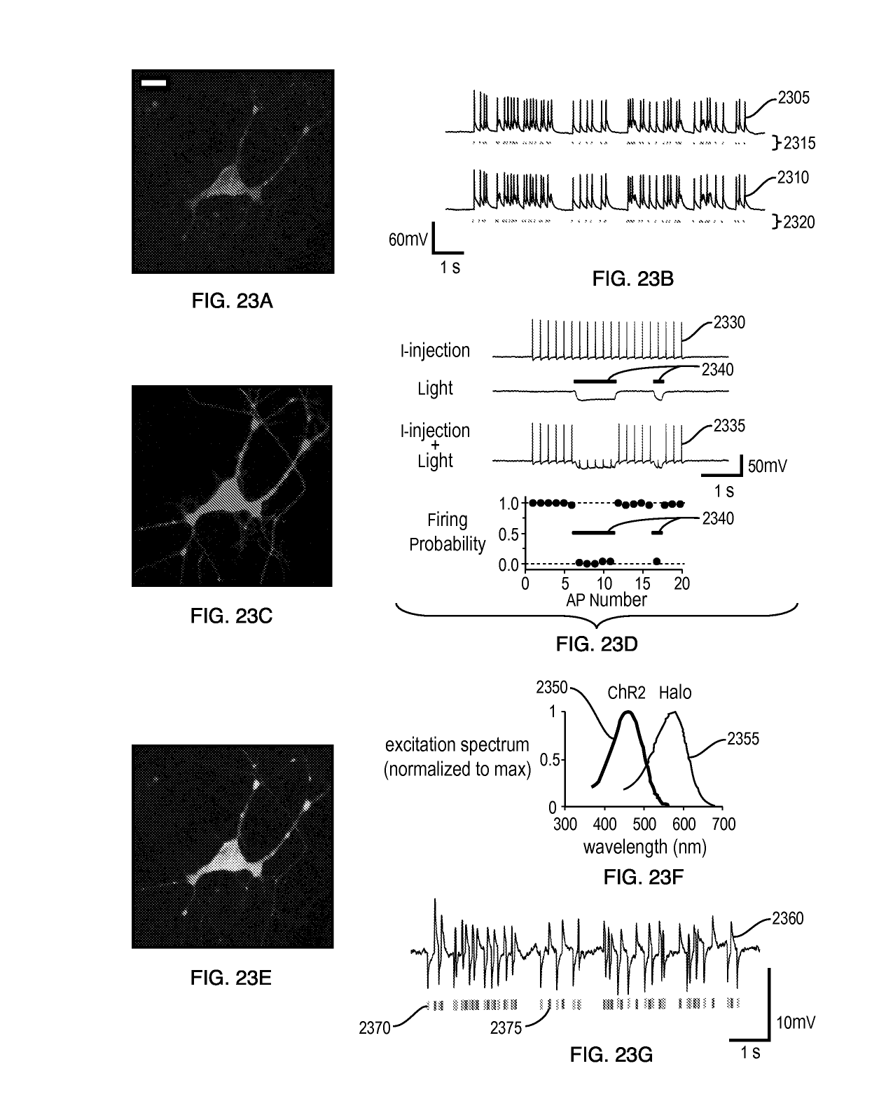

As shown in FIGS. 23A-G, neuron-expressing channelrhodopsin-2 is fused to mCherry (FIG. 23A; bar, 20 μm) and halorhodopsin is fused to GFP (FIG. 23C). The overlay is shown in FIG. 23E. In FIG. 23B, Poisson trains of spikes 2305, 2310 are elicited by pulses of blue light 2315, 2320 in two different neurons. In FIG. 23D, light-driven spike blockade 2330 is depicted for a representative hippocampal neuron, and another light-driven spike blockade 2335 is shown for a population of neurons (n=7). Neuronal firing was induced by pulsed somatic current injection (300 pA, 4 ms). Hyperpolarization was induced by periods of yellow light 2340. As seen for spike blockade 2335, yellow light 2340 drives Halo to block neuron spiking, leaving spikes elicited during periods of darkness intact. In FIG. 23F, action spectrum 2350 for ChR2 overlaid with absorption spectrum 2355 for N. pharaonis halorhodopsin. FIG. 23G depicts hyperpolarization and depolarization events 2360 induced in a representative neuron by a Poisson train of alternating pulses (10 ms) of yellow 2370 and blue 2375 light.

In an illustrative implementation, a 3-D array of LEDs capable of targeting arbitrary brain structures enables activation, silencing, and the resculpting of activity throughout multiple brain regions. This 3D array provides a probe that is inexpensive, compact, power-efficient, self-contained, and capable of exerting optical control over a large region of brain tissue while causing a minimum of brain damage. In illustrative implementations, the present invention may employ such 3-D arrays of compact, inexpensive, long-lasting, and bright light-emitting diodes (LEDs), capable of targeting arbitrary brain regions for activation or silencing. Unlike an optical fiber, which can only deliver light to a single point in the brain per penetrating device, a linear array of LEDs inserted into the brain can deliver light to points in the brain up and down the probe. In an illustrative embodiment, the device is compact (human DBS electrodes are <1.5 mm wide), is capable of delivering bright light into deep brain structures, and does not heat the brain beyond an acceptable limit (typically, 1° C.). One efficient way to implement a 3-D array of LEDs is to create a 2-D array of 1-D probes.

In a prototype implementation, fiber arrays were implemented using individual lasers or LEDs, but arrays of vertical cavity surface emitting lasers (VCSELs) or other optical sources may work just as well. If in the future xenon bulbs, halogen lamps, incandescent bulbs, or other light sources become miniaturized enough to fit in, they may also be suitable for use with some implementations of the present invention (with filters on the bulbs). A particularly appealing way to modulated LED power with a simple circuit is to pulse width modulate (PWM) the LED. A particularly simple wireless method is just to attach an LED to an inductor, which will then be remotely powerable.

In the description and implementation of the present invention, it will be understood by one of ordinary skill in the art that each of the variations on the component parts of the invention are swappable with any of the other variations. Similarly, when a use of the present invention is described with respect to a particular tissue or body part, it will be understood by one of ordinary skill in the art that the invention can be used in a similar manner for other body parts and tissues. For example, if a use is described is for the “brain,” then it may similarly used in any other bodily tissue (e.g., peripheral nerve, pancreas, etc.). As another example, if it is described how to affix something to the skin, it may similarly be used in dealing with muscle and other tissues as well. The terms light source or LED are also used interchangeably, as they all have similar functionality in the context of the present invention. The light produced by the source may be visible light, infrared, spectrally complex, or any other type of light found to be suitable for the particular application. Further, while the use of wireless communications is described herein chiefly in the language of RF transceiving, it will be clear to one of skill in the art that any kind of wireless communication, such as, but not limited to, ultrasound or light may also be advantageously used in the present invention.

In an illustrative implementation, LEDs are placed on thin sheets of metal (which can be curved if desired), and the 1-D probes can be made as long or as short as desired, using the rapid-prototyping tools here described. Accordingly, optical cochlear prostheses, vestibular prostheses, and peripheral nerve prostheses may be rapidly deployed. Although the above optical probe may be as small or smaller than comparable human devices (e.g., DBS electrodes), even smaller versions capable of controlling neurons in animals as small as a juvenile mouse may be constructed.

In an illustrative implementation, an optional dichroic, beamsplitter, or other equivalent optical part known in the art may be attached to a fiber to couple two different light sources (such as, but not limited to, a blue LED and a yellow LED, or a blue laser and a yellow laser) into the fiber, so that the target cells at the end of the fiber can be more easily activated and deactivated by two different colors of light. This may further be generalized to a series of cascaded dichroics, capable of coupling more than two colors of light into the same fiber.

In order to develop a reliable testbed for activation and silencing of genetically-sensitized neurons with blue and yellow light, a prototype system capable of coupling two strong lasers into a single optical fiber was constructed. Two diode-pumped solid state (DPSS) lasers, a yellow (593 nm) 30 mW laser and a blue (473 nm) 200 mW laser (Aixiz Int'l.) were employed. These lasers can be activated for milliseconds at a time when triggered by TTL pulses.

In an illustrative implementation, blue and yellow light is sent down a fiber to be implanted inside the head, for bi-directional control of a single kind of neuron at a single site within the brain.

In an illustrative implementation, two mirrors on adjustable gimbals steered beams from blue and yellow lasers into fiber collimator (Thorlabs F810SMA), with the assistance of dichroic element (Chroma) that reflects blue light and passes yellow light, thus bringing the two laser beams into collinearity. SMA-terminated multimode fiber, 200 microns in diameter and capable of passing light throughout the visible range, was inserted into collimeter. The free end of fiber, highly polished, could then be inserted into the brain of an experimental animal. In the prototype embodiment, the power coming out of the fiber approached 800 mW/mm² in the blue, sufficient to stimulate ChR2, and 110 mW/mm² in the yellow, sufficient to stimulate Halo. In a mouse, a thin polyimide tube trimmed to the correct length, and inserted into the brain to stereotactically target the brain region of interest, serves easily as a guide cannula for inserting the fiber; gluing a thin washer onto the fiber helped prevent insertion of the fiber into the brain beyond the desired point.

In one embodiment, an optional steerable element, such as, but not limited to, a galvanometer, an acousto-optic deflector, a MEMS mirror, or other steering device is employed on one or both ends of the fiber, to direct light in a controlled way, thus enabling locally selective targeting of the light to specific areas of the tissue, preferably with as few moving parts as possible.

To activate channelrhodopsin-2 and halorhodopsin molecules in mammalian neurons may require light of the appropriate color at a radiant flux of 10 mW/mm² or greater, for maximal activation. A radiant flux of 1 mW/mm² will activate approximately 50% of the molecules, and a radiant flux of 0.1 mW/mm² will activate very few of the molecules. Since light is absorbed and scattered as it passes through tissue, this means that relatively bright light sources are needed to activate neurons embedded in tissue; furthermore, it implies that for any given light source, there will be heterogeneity in the power that reaches neurons at various distances from the light source.

In an illustrative implementation, a 280 μm×280 μm blue (460 nm) C460EZ290-S2400 LED from Cree [Cree, CPR3CQ.pdf, (2007)] and the 305 μm×305 μm yellow (590 nm) HWFR-B317 LED from Lumileds [Lumileds, DS42.PDF (2007)], were employed. The blue LED has a 1% contour envelope that resembles an ellipse 1.6 mm long and 1.4 mm wide; at a peak power of 24 mW, the 1% contour equates to ˜3 mW/mm² radiant flux—an intensity easily sufficient to activate ChR2. Similarly, for the yellow LED, the 1% contour envelope resembles an ellipse 1.7 mm long and 1.5 mm wide, which at its peak power of 86 mW equates to a radiant flux of ˜15 mW/mm² for yellow light, an intensity sufficient to activate the majority of Halo molecules. Of course, to illuminate smaller volumes, the light power can always be decreased. Standard 300-micron LEDs should be able to illuminate brain volumes across a broad scale, all the way from cubic microns to several cubic millimeters.

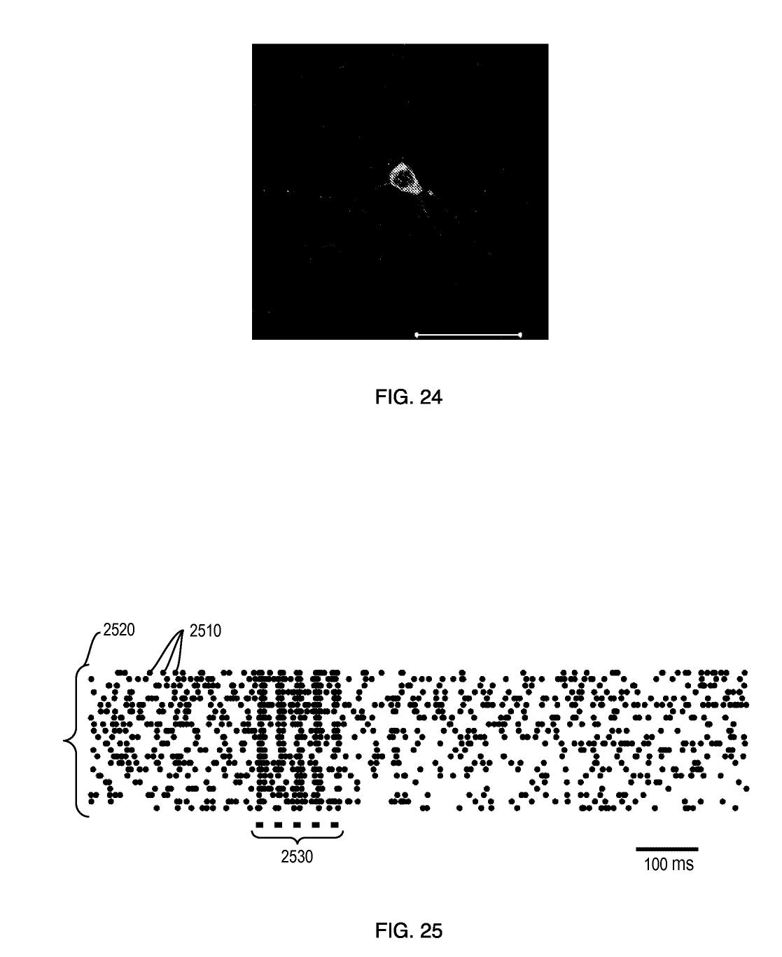

An illustrative implementation of the present invention has been prototyped and experimentally verified using several testing methodologies. The ability to make specific neurons in the brain light-sensitive, using a viral approach and control of neural activity in the cortex of the non-human primate were demonstrated. In these tests, one experiment assessed intact tissue expression in mouse. Mammalian codon-optimized forms of channelrhodopsn-2 and halorhodopsin, abbreviated as hChR2 and Halo, have been previously developed. These genes were inserted into a lentiviral vector that allows cloning in different promoters, or DNA regulatory elements, upstream of the gene of interest. For example, the CaMKII promoter targets predominantly excitatory neurons. FIG. 24 is an image of neurons in the mouse brain expressing Halo-GFP under the CaMKII promoter, which preferentially labels excitatory neurons. In FIG. 24, the Scalebar represents 50 μm. A pipeline was developed for obtaining promoters by cloning them out of bacterial artificial chromosomes (BACs) and then inserting them into lentiviral plasmids upstream of hChR2 or Halo. Small virus test batches are then created, and promoter strength and selectivity in the mouse brain are subsequently rapidly screened. By screening promoters in a wholesale fashion, it is possible to identify and validate new candidate promoters for targeting specific cell types.

For this experiment, replication incompetent lentiviruses were produced via triple transfection of plasmids containing the promoter and gene of interest (e.g., F(CK)-Halo-GFP or F(CK)-ChR2-GFP), the viral helper plasmid (pA8.91), and the pseudotyping plasmid (pMD2.G, encoding the coat protein VSV-G). Briefly, HEK293FT cells (Invitrogen) were plated onto four T175 flasks in D10 medium (comprising DMEM+10% FBS+1% pen-strep, 1% sodium pyruvate, and 1% sodium bicarbonate). At 100% confluence, cells were transfected with DNA using Fugene: 22 micrograms of plasmids containing the promoter and gene of interest, 15 micrograms of pΔ8.91, and 5 micrograms of pMD2.G, were mixed with 132 microliters of Fugene 6 and 4.5 mL of MEM, prepared according to the instructions of the manufacturers of Fugene. 24 hours later, the cells were washed with D10 and then given 30 mL of virus production media (comprising Opti-MEM w/GlutaMAX-I+1% pen-strep, 1% sodium pyruvate, and 1% sodium bicarbonate). 48 hours later, the supernatant was harvested, filtered through a 0.45 micron filter flask (pre-wetted with D10), and then the filtrate was ultracentrifuged over a 20% sucrose cushion at 22000 rpm in a SW-28 rotor for 2 hours at 4° C. The pellet was then resuspended in 30 microliters of PBS over a period of several hours, and aliquotted the virus for storage at −80° C.

Viruses were tested for efficacy in sensitizing specific neuron types to being activated/silenced by light by being injected into the cerebral cortex of mice. Mice were anesthetized with 1.25-2% isoflurane and placed into a custom stereotax. A dental drill was used to make a small craniotomy, through which 1-2 microliters of virus was injected into the cerebral cortex of the mouse brain. The virus was injected through a pulled borosilicate glass pipette (tip ˜5 microns wide; shank ˜4 mm long), pulled with a Sutter P-97 puller. This glass pipette was connected to a Hamilton syringe (placed in a syringe pump, from Harvard Apparatus) via a thin plastic tube filled with silicone oil. Virus infusion was carried out by actuating the Harvard Apparatus pump to inject slowly (e.g., 0.1 microliters per minute) over a period of 20 minutes. After the viral payload was delivered, 10-20 minutes was allowed to pass in order for the virus to diffuse away from the site of injection before withdrawing the pipette at a slow rate (e.g., 2 mm/min). The scalp of the mouse was then sealed with Vetbond, and the animal administered buprenorphine and returned to its home cage.

One to four weeks after virus injection, acute slices of brain tissue were cut in order to assess the targeted cells for strength and specificity of gene expression. Briefly, mice were anesthetized with isoflurane and decapitated, and the brains were removed to ice cold cutting solution (87 mM NaCl, 25 mM NaHCO₃, 25 mM glucose, 75 mM sucrose, 2.5 mM KCl, 1.25 mM NaH₂PO₄, 0.5 mM CaCl₂ and 7 mM MgCl₂, bubbled with 95% O₂/5% CO₂). Time between death and completion of brain removal was less than 1 minute. Brains were then blocked and glued to a dish for cutting with a vibrating tissue slicer (Leica VT1000S) into sections 240 microns thick. Slices were incubated at 35° C. for 30 minutes, then stored at room temperature. Slices were acutely examined in physiological saline (125 mM NaCl, 25 mM NaHCO₃, 25 mM glucose, 2.5 mM KCl, 1.25 mM NaH₂PO₄, 2 mM CaCl₂, and 1 mM MgCl₂). At this time, cells were examined electrophysiologically through whole-cell patch clamp (access resistance, 4-8 megaohms), conducted with a Molecular Devices Multiclamp 700B setup.

In a second test, optically-elicited spike trains were obtained from a primate cortex.

A craniotomy and plastic chamber implantation was performed over the frontal eye field (FEF) region of the cortex of a rhesus macaque that had been prepared with a headpost, and trained to fixate upon a point. Recordings were made with tungsten microelectrodes (1-2 megaohm impedance) that were slowly advanced through cortical tissue until single units were isolated, acquiring data via a Multichannel Acquisition Processor from Plexon, Inc. Spike recordings were filtered, and spikes isolated using an interactively-set threshold. To facilitate precise and repeatable optical stimulation and recording, a recording chamber insert with a grid of evenly-spaced holes was designed and fabricated, in order to facilitate optimal placement of electrodes and optical fibers.

As a test case, a male rhesus macaque was prepared for cortical recording in the frontal eye field (FEF) area of cortex, as predicted by MRI scans and validated with electrophysiology. Surgically injection of 1-4 μL of virus expressing ChR2-GFP under the CaMKII promoter into a site within FEF was undertaken, using the equipment described previously. After a 2-week waiting period for the virus to express, and a polished, 200 micron-thick optical fiber whose other end was fiber-coupled to a 200 mW blue (473 nm) laser was inserted. After optimizing the recording, the laser was activated to fire brief pulses of blue light by delivering 10 ms-long TTL pulses separated by 20 ms pauses (i.e., 33.3 Hz stimulation), and elicited trains of well-timed spikes.

FIG. 25 is a raster plot showing the experimentally-obtained occurrences of spikes elicited from an excitatory neuron expressing ChR2 in a monkey cortex in response to a brief train of blue light stimulation. As shown in FIG. 25, spikes (black dots) 2510, in 22 consecutive spike trains (each row of black dots) 2520 were elicited in response to blue light stimulation. Each horizontal row 2520 reflects one recording of the response to five blue pulses 2530 of light, each lasting 10 ms and separated from the next by 20 ms (i.e., 33.3 Hz stimulation rate). This result demonstrates, for the first time, that both the genetic targeting of neurons, and the use of light to activate them, can work successfully in the macaque cortex.

In illustrative implementations, the present invention may be advantageously employed for the purpose of activating or silencing circuits in a targeted way. In illustrative implementations, the device is capable of activating and silencing neurons at the scale of microns to millimeters, and is inexpensive, reliable, and easy to use. Effective viral targeting of excitatory neurons in the intact brain has been demonstrated using the present invention. The experimental results have demonstrated for the first time that optical neural control technologies are capable of working in the intact primate brain, a major milestone in the quest to enable novel human therapies. In illustrative implementations, the present invention may be advantageously used to understand how to control neural circuits to compensate for the loss or alteration of specific cell types, as often occur in neurological and psychiatric diseases ranging from obesity, to pain, to Parkinson's, to epilepsy.

### Neural Control:

According to principles of this invention, light-sensitive membrane proteins such as the depolarizing cation channel Channelrhodopsin-2 (here abbreviated ChR2) and the hyperpolarizing ion pumps Halorhodopsin and its derivatives and Archaerhodopsin (here abbreviated Arch), may be used to activate or silence neurons with millisecond timescale precision. These proteins may be selectively expressed in neural populations defined by neuroanatomical boundaries, cell types, pathways, and activation patterns through standard viral or transgenic methods, enabling precise bidirectional control of defined neural circuit elements on the timescale of neural computation.

In exemplary implementations of this invention, assays may be performed with subjects that may move freely within a behavioral arena. This is advantageous, because many cognitive functions in rodents, such as emotion, attention, working memory, and spatial learning, are assessed through behaviors which require the animal to have free movement throughout a behavioral arena.

In exemplary implementations of this invention, readout is used simultaneously with the lightguide arrays to probe the effect of optical modulation on neural circuits. Different types of readout may be used. Here are three examples: First, behavioral monitoring technologies, including real-time automated behavioral classification algorithms, can be used to gauge the net effect of optical perturbation on behaviors of interest. Second, electrophysiological recording, using probes such as tetrodes, microwire arrays, or linear electrode arrays, can measure the response of individual neurons and large neural ensembles which receive direct optical perturbation or participate in the same neural circuit as optically perturbed circuit elements. Third, whole-brain imaging methods such as fMRI and PET, which may necessitate head-fixation to resolve images taken over long timescales, may nevertheless utilize the multisite 3-dimensionality of the lightguide arrays to assess brain-wide responses to perturbation of specific neural circuit elements, as well as generate lists of targets which respond to certain perturbations, for potential screening in relation to behaviors known to rely on specific neural circuit elements.

According to principles of this invention, neural circuit elements may be targeted to express light-activated membrane proteins in a number of ways. Viral vectors (such as lentiviruses, adeno-associated viruses (AAVs), and herpes-simplex virus (HSVs)) containing a DNA payload encoding for the desired protein, may be injected directly into neuoranatomical regions of interest. Neuroanatomical boundaries such as white matter tracts often limit the spread of viral particles, resulting in the expression of light-activated membrane proteins exclusively in neuroanatomical regions of interest. Activation of specific neuroanatomical regions can also be achieved by limiting the number of injected viral particles to restrict expression of light-activated membrane proteins within a chosen radius from the injection site, or limiting the power of optical sources inserted in the brain so that light levels outside the region of interest are not great enough to excite the light-activated membrane proteins. Cell-type specificity for expression of light-activated membrane proteins may be achieved by inclusion of the appropriate promoter upstream of the code for the protein in the DNA payload of the viral vector. Transgenic mice may also be produced to express light-activated membrane proteins under specific promoters. Alternatively, transgenic mice expressing Cre-recombinase under specific promoters may be injected with virus in which the DNA encoding for light-activated membrane proteins is flanked by lox-p sites and inserted backwards into the DNA payload, such that only cells expressing Cre-recombinase cleave the DNA at the lox-p sites and reverse the orientation of the DNA for the light-activated membrane protein. Additionally, pathways between neural circuit elements may be selectively targeted by injecting viruses which travel through anterograde or retrograde connections, such as viruses containing the rabies coat protein. Or viruses may be injected in one region of the brain, but their axon terminals may be stimulated in another region of the brain, such that only connections between the circuit elements in those two regions are targeted. Additionally, expressing light-activated membrane proteins under promoters for immediate early genes such as c-fos may be used to selectively target neural circuit elements which display specific patterns of activity that result in the expression of immediate early genes.

Surgery may be performed under general anesthesia to inject virus into a brain region containing a neural circuit element of interest. For example, the surgery may be performed as follows: An incision is made in the skin on top of the head, the location of the region is determined by landmarks on the skull and stereotaxic coordinates derived from a brain atlas, a craniotomy (often ˜300 microns wide, but may be many millimeters wide) is drilled into the skull, a needle (˜200 micron diameter) containing virus (often ˜1 microliter, but volumes from 0.5-5 microliters may be feasible) is lowered into the brain to reach the target brain region, and the virus is slowly (over the course of ˜10-20 minutes) injected into the brain with the aid of a syringe pump connected to the needle through tubing containing an incompressible liquid such as silicone oil. To implant a lightguide array, a similar surgery (or an adjunct to the same surgery) is performed in which craniotomies are drilled into the skull to allow entrance for the lightguides, and the lightguide array is lowered into place on a stereotaxic arm. The array is cemented to the skull via several small screws implanted in the skull around the lightguide array.

In illustrative implementations of this invention, lightguide arrays may be powered and controlled with wired or wireless circuitry, programmed with a simple interface. When used in a wired configuration, a system of pulleys can be used to counterbalance the added weight of the wires to keep the mouse unencumbered. This system may also include a commutator to allow the mouse to freely turn in the behavioral arena without twisting the cables it is attached to. These cables may include fluid cooling lines to prevent heat produced by light sources in the arrays from detrimentally heating the mouse skull; however, sufficiently short activation paradigms do not require such cooling.

In some implementations of this invention, 3-dimensional light guide arrays are implanted in, or on top of, or adjacent to, a mammalian brain to perturb defined neural circuit elements to screen for their role in a mammal's behavior.

### Light-Activated Protein Pumps:

In illustrative implementations, this invention comprises a method for adjusting the voltage potential or pH of, or cause proton release from, cells, subcellular regions, or extracellular regions. A gene encoding for a light-driven proton pump is incorporated into at least one target cell or region. A proton pump operates in response to a specific wavelength of light. The target cell or region to the specific wavelength of light is exposed to light, causing the voltage potential adjustment, pH adjustment, or proton release. The proton pump may be a microbial rhodopsin, in particular derived from the halorubrum genus of archaeabacteria, or be derived from leptosphaeria maculans, P. triticirepentis, and S. scelorotorium. The voltage potential of the target cell or region may adjusted until it is hyperpolarized in order to achieve neural silencing. Light-activated proton pumps responsive to different wavelengths of light may be used together to achieve multi-color neural silencing.

In illustrative implementations, light-activated proton pumps modify cell parameters, including transmembrane potential and/or pH of cells, their sub-cellular regions, and their local environment. In particular, the use of outwardly rectifying proton pumps can hyperpolarize cells by moving positively charged ions from the cytoplasm to the extracellular environment. Under specific conditions, their use can increase the intracellular pH or decrease the extracellular pH.

In one aspect, the present invention shows that members of the class of light-driven outward proton pumps can mediate very powerful, safe, multiple-color silencing of neural activity. The gene archaerhodopsin-3 (“Arch”) from Halorubrum sodomense enables near-100% silencing of neurons in the awake brain when virally expressed in mouse cortex and illuminated with yellow light. Arch mediates currents of several hundred picoamps at low light powers, and supports neural silencing currents approaching 900 pA at light powers easily achievable in vivo. In addition, Arch spontaneously recovers from light-dependent inactivation, unlike light-driven chloride pumps that enter long-lasting inactive states in response to light. These properties of Arch are appropriate to mediate the optical silencing of significant brain volumes over behaviourally-relevant timescales. Arch function in neurons is well tolerated because pH excursions created by Arch illumination are minimized by self-limiting mechanisms to levels comparable to those mediated by channelrhodopsins or natural spike firing.

In another aspect of the present invention, the blue-green light-drivable proton pump from the fungus Leptosphaeria maculans⁴ (“Mac”) can, when expressed in neurons, enable neural silencing by blue light, thus enabling, alongside other developed reagents, the potential for independent silencing of two neural populations by blue vs. red light. Light-driven proton pumps thus represent a high-performance and extremely versatile class of “optogenetic” voltage and ion modulator, which will broadly empower new neuroscientific, biological, neurological, and psychiatric investigations.

Arch and Mac are each an optical neural silencing reagent. Arch and Mac are also each a light-driven proton pump, which operates without the need for exogenous chemical supplementation in mammalian cells. The efficacy of these proton pumps is surprising, given that protons occur, in mammalian tissue, at a millionfold-lower concentration than the ions carried by other optical control molecules. This high efficacy may be due to the fast photocycle of Arch, but it may also be due to the ability of high-pKa residues in proton pumps to mediate proton uptake. Proton pumping is a self-limiting process in neurons, providing for a safe and naturalistic form of neural silencing. Proton pumps recover spontaneously after optical activation, improving their relevance for behaviourally-relevant silencing over the class of halorhodopsins. Proton pumps exist with a wide diversity of action spectra, thus enabling multiple-color silencing of distinct neural populations.

In illustrative implementations, the expression, in genetically-targeted cells, of certain classes of genes encoding for light-driven proton pumps enables powerful hyperpolarization of cellular voltage in response to pulses of light. These pumps can be genetically-expressed in specific cells (for example, but not limited to, by using a virus) and then used to control cells in intact organisms (including, but not limited to, humans), as well as cells in vitro, in response to pulses of light. The magnitude of the current that can be pumped into cells expressing these pumps, upon exposure to light, is up to 16× greater than that of state-of-the-art pumps (e.g., Halo/NpHR). Because the pumps of the present invention have different activation spectra from one another and from the state of the art natural gene products (e.g., Halo/NpHR), they also enable multiple colors of light to be used to hyperpolarize different sets of cells in the same tissue by expressing pumps with different activation spectra genetically in different cells and then illuminating the tissue with different colors of light.

In illustrative implementations, two classes of light-activated proton pumps are used to hyperpolarize excitable cells: Microbial rhodopsins, such as the Halorubrum sodomense gene for archaerhodopsin-3 (herein abbreviated “Arch”) and Halorubrum strain TP009 gene for archaerhodopsin-TP009 (herein abbreviated “ArchT”), and eukaryotic proton pumps, such as leptosphaeria maculans (herein abbreviated “Mac”), P. triticirepentis, and S. sclerotorium rhodopsins. These proton pumps can also be used to modify the pH of cells and/or to release protons as chemical transmitters.

As used herein, the following terms expressly include, but are not to be limited to:

“Proton pump” means an integral membrane protein that is capable of moving protons across the membrane of a cell, mitochondrion, or other subcellular compartment.

In illustrative implementations, microbial rhodopsins can be used in mammalian cells without need for any kind of chemical supplement, and in normal cellular environmental conditions and ionic concentrations. In illustrative implementations, archaerhodopsins (proton pumps from Halorubrum, such as the Halorubrum sodomense gene for archaerhodopsin-3 (herein abbreviated “Arch”) and Halorubrum strain TP009 gene for archaerhodopsin-TP009 (herein abbreviated “ArchT”) encode for genes that, in humanized or mouse-optimized form, enable hyperpolarizations significantly larger than what has been discovered before.

In illustrative implementations, Leptosphaeria maculans rhodopsin responds strongly to blue light, and since other opsins identified to hyperpolarize cells respond to green, yellow, or reddish light, Leptosphaeria maculans rhodopsin can be expressed in a separate population of cells from a population of cells expressing one of these other opsins, thus allowing multiple colors of light to be used to silence these two populations of cells or neuronal projections from one site at different times.

An illustrative implementation of the present invention was reduced to practice in the laboratory by genetically expressing these molecules in excitable cells, illuminating the cells with light, and demonstrating rapid hyperpolarization of these cells in response to light, as well as rapid release from hyperpolarization upon cessation of light. The ability to controllably alter intracellular pH with light was also demonstrated, as was the ability to control membrane conductance bi-directional control via single molecule type that can depolarize or hyperpolarize a neuron with different colors of light or different light intensities. In illustrative implementations, light is used to control cellular functions in vivo (including in the non-human primate, which demonstrates pre-clinical enablement in humans) and in vitro

Most proton pumps do not express well in mammalian cells. It was therefore necessary to screen a great many proton pumps in order to identify the class of microbial archaerhodopsins that was determined in the present invention to function better in mammalian cells than did other classes of proton pumps.

In order to identify the opsins of an illustrative implementation of the present invention, type I microbial opsins from archaebacteria, bacteria, plants, and fungi were screened for light-driven hyperpolarizing capability. Table 1 lists the modular screening candidates, including abbreviations, molecule classification, species of origin, and GenBank accession number (non-codon optimized). In Table 1, major molecule types are defined as “bacteriorhodopsins” or “rhodopsins” for outwardly rectifying proton pumps and “halorhodopsins” for inwardly rectifying chloride pumps. Sub-classifications of molecule type are determined by kingdom and/or genus of species of origin (e.g. archaerhodopsin=proton pump from halorubrum genus of halobacteria/haloarchaea).

TABLE 1

| Abbrevi- | | Species of | GENBANK |
|---|---|---|---|
| ations | Molecule class | origin | Accession |
| Halo, NpHR, | halorhodopsin | Natronomas | ABQ08589 |
| pHR | | pharaonis | |
| sHR, HR | halorhodopsin | Halobacterium | NP_279315 |
| | | salinarum | |
| aHR-3 | archaehalorhodopsin | Halorubrum | BAA75202 |
| | | sodomense | |
| aHR-1, | archaehalorhodopsin | Halorubrum | CAA49773 |
| SGHR | | aus-1 (sp. SG1) | |
| cHR-3 | cruxhalorhodopsin | Haloarcula | BAA06679 |
| | | vallismortis | |
| cHR-5 | cruxhalorhodopsin | Haloarcula | AAV46572 |
| | | marismortui | |
| SquareHOP | square | Haloquadratum | CAJ53165 |
| | halorhodopsin | walysbyi | |
| dHR-1 | deltahalorhodopsin | Haloterrigena | BAA75201 |
| | | sp. Arg-4 | |
| SalHO, | bacterial | Salinibacter | AAT76430 |
| SRU_2780 | halorhodopsin | ruber | |
| pop | fungal opsin | Podospora | XP_001904282 |
| | related protein | anserina | |
| | | (DSM980) | |
| nop-1 | fungal opsin | Neospora | XP_959421 |
| | related protein | crassa | |
| Mac, LR, | Fungal opsin | Leptosphaeria | AAG01180 |
| Ops | related protein, | maculans | |
| | bacteriorhodopsin | | |
| Arch, aR-3 | archaerhodopsin | Halorubrum | BAA09452 |
| | | sodomense | |
| BR | Bacteriorhodopsin | Halobacterium | CAA23744 |
| | | salinarum | |
| cR-1 | Cruxrhodopsin | Haloarcula | BAA06678 |
| | | argentinensis | |
| | | (sp. arg-1 | |
| gPR, | Proteorhodopsin | γ - | AAG10475 |
| BAC31A8 | | proteobacterium | |
| | | BAC31A8 | |
| bPR, | proteorhodopsin | γ- | Q9AFF7 |
| Hot75m4 | | proteobacterium | |
| | | Hot75m4 | |
| Ace, AR | Algal | Acetabularia | AAY82897 |
| | bacteriorhodopsin | acetabulum | |

Mammalian codon-optimized genes were synthesized, cloned into GFP-fusion expression vectors, and transfected into cultured neurons. Opsin photocurrents and cell capacitance-normalized photocurrent densities were then measured under stereotyped illumination conditions, as well as opsin action spectra (photocurrent as a function of wavelength). The action spectra of screened candidates are presented in Table 2. In Table 2, reported peaks and full-width at half-maximum values are from second order Gaussian fits, in order to account for the characteristic “shoulder” of rhodopsins. “Spectral Screen Normalization Factor” accounts for differences in measured photocurrents due to varying excitation efficiencies via use of limited bandpass illumination filters (575±25 nm, 535±25 nm) for all molecules in the screen. All data reported was measured in neurons, except for Ace (Acetabularia acetabulum bacteriorhodopsin homolog), which was measured in HEK 293FT cells, in order to obtain a precise spectrum given the very low currents observed in neurons.

TABLE 2

| | | | | | Spectral Screen |
|---|---|---|---|---|---|
| | | | | Primary Peak ± | Normalization |
| | | | | FWHM | Factor, relative |
| | | | | (nm), | to Halo (see |
| | | Species of | GENBANK | second order | Experimental |
| Abbreviations | Molecule class | origin | Accession | Gaussian fit | Procedures) |
| Halo, NpHR, | halorhodopsin | Natronomas | ABQ08589 | 586 ± 52 | 1 |
| pHR | | pharaonis | | | |
| sHR, HR | halorhodopsin | Halobacterium | NP_279315 | No measured | N/A |
| | | salinarum | | photocurrent | |
| aHR-3 | archaehalorhodopsin | Halorubrum | BAA75202 | 586 ± 63 | 1.18 |
| | | sodomense | | | |
| aHR-1, SGHR | archaehalorhodopsin | Halorubrum | CAA49773 | No measured | N/A |
| | | aus-1 (sp. SG1) | | photocurrent | |
| cHR-3 | cruxhalorhodopsin | Haloarcula | BAA06679 | 592 ± 58 | 1.17 |
| | | vallismortis | | | |
| cHR-5 | cruxhalorhodopsin | Haloarcula | AAV46572 | 594 ± 52 | 1.10 |
| | | marismortui | | | |
| SquareHOP | square halorhodopsin | Haloquadratum | CAJ53165 | ¹⁴⁴³²⁷¹⁹¹⁹No | N/A |
| | | walysbyi | | measured | |
| | | | | photocurrent | |
| dHR-1 | deltahalorhodopsin | Haloterrigena | BAA75201 | No measured | N/A |
| | | sp. Arg-4 | | photocurrent | |
| SalHO, | bacterial | Salinibacter | AAT76430 | 582 ± 71 | 1.12 |
| SRU_2780 | halorhodopsin | ruber | | | |
| Pop | fungal opsin related | Podospora | XP_001904282 | No measured | N/A |
| | protein | anserina | | photocurrent | |
| | | (DSM980) | | | |
| nop-1 | fungal opsin related | Neospora | XP_959421 | No measured | N/A |
| | protein | crassa | | photocurrent | |
| Mac, LR, Ops | fungal opsin related | Leptosphaeria | AAG01180 | 550 ± 69 | 0.94 |
| | protein, | maculans | | | |
| | bacteriorhodopsin | | | | |
| Arch, aR-3 | archaerhodopsin | Halorubrum | BAA09452 | 566 ± 66 | 1.08 |
| | | sodomense | | | |
| BR | bacteriorhodopsin | Halobacterium | CAA23744 | 572 ± 75 | 1.26 |
| | | salinarum | | | |
| cR-1 | cruxrhodopsin | Haloarcula | BAA06678 | 557 ± 67 | 1.23 |
| | | argentinensis | | | |
| gPR, | proteorhodopsin | γ- | AAG10475 | No measured | N/A |
| BAC31A8 | | proteobacterium | | photocurrent | |
| | | BAC31A8 | | | |
| bPR, Hot75m4 | proteorhodopsin | γ- | Q9AFF7 | No measured | N/A |
| | | proteobacterium | | photocurrent | |
| | | Hot75m4 | | | |
| Ace, AR | algal | Acetabularia | AAY82897 | 505 ± 57 | 0.98 |
| | bacteriorhodopsin | acetabulum | | | |

From this information, the photocurrent density for each opsin was estimated at its own spectral peak. For comparison purposes, an earlier-characterized microbial opsin was included, the Natronomonas pharaonis halorhodopsin (Halo/NpHR), a light-driven inward chloride pump capable of modest hyperpolarizing currents. Archaerhodopsin-3 from Halorubrum sodomense (Arch/aR-3), hypothesized to be a proton pump, generated large photocurrents in the screen, as did two other proton pumps, the Leptosphaeria maculans opsin (Mac/LR/Ops) and cruxrhodopsin-1 (albeit less than that of Arch). All light-driven chloride pumps assessed had lower screen photocurrents than these light-driven proton pumps.

FIG. 1 is a comparison graph depicting optical neural silencing by light-driven proton pumping, as revealed by a cross-kingdom functional molecular screen. Shown in FIG. 1 are screen data showing outward photocurrents (left y axis, black bars), photocurrent densities (right y axis, grey bars), and action spectrum-normalized photocurrent densities (right y axis, white bars), measured by whole-cell patch-clamp of cultured neurons under screening illumination conditions (575±25 nm, 7.8 mW/mm² for all except Mac/LR/Ops, gPR, bPR and Ace/AR, which were 535±25 nm, 9.4 mWmm²; n=4-16 neurons for each bar). Data are mean+/−standard error. Full species names from left to right: Natronomonas pharaonis, Halobacterium salinarum, Halorubrum sodomense, Halorubrum species aus-1, Haloarcula vallismortis, Haloarcula marismortui, Haloquadratum walsbyi, Haloterrigena species Arg-4, Salinibacter ruber, Podospora anserina, Neurospora crassa, Leptosphaeria maculans, Halorubrum sodomense, Halobacterium salinarum, Haloarcula species Arg-1, uncultured gamma-proteobacterium BAC31A8, uncultured gamma-proteobacterium Hot75m4 and Acetabularia acetabulum.

Arch and ArchT have demonstrable photocurrent generation at many illumination wavelengths. Arch is a yellow-green light sensitive opsin that appears to express well on the neural plasma membrane. Arch-mediated currents exhibited excellent kinetics of light-activation and post-light recovery. Upon illumination, Arch currents rose with a 15%-85% onset time of 8.8±1.8 ms (mean±standard error (SE) reported throughout, unless otherwise indicated; N=16 neurons), and after light cessation, Arch currents fell with an 85%-15% offset time of 19.3±2.9 ms. Under continuous yellow illumination, Arch photocurrent declined, as did the photocurrents of all of the opsins in the screen. However, Arch spontaneously recovered function in seconds, more like the light-gated cation channel channelrhodopsin-2 (ChR2).

The observed behavior of Arch is unlike all of the other halorhodopsins screened, including products of halorhodopsin site-directed mutagenesis aimed at improving kinetics, which after illumination remained inactivated for long periods of time (e.g., tens of minutes, with accelerated recovery requiring additional blue light). Table 3 presents action spectrum and spontaneous recovery to active pumping state in the dark for N. pharaonis halorhodopsin (Halo, NpHR) point mutants examined in HEK293FT cells. In Table 3, reported peaks and full-width at half-maximum values are from second order Gaussian fits, in order to account for the characteristic “shoulder” of rhodopsins. The column “Primary predicted outcome of mutation” lists hypothesized outcomes as to what parameters of molecular performance might be expected to change, for each mutation. The term “Recovery” refers to spontaneous recovery of the active pumping state in the dark over a timescale of seconds, which holds for Arch and ChR2 but not for Halo. “Recovery” candidate residues were targeted based on their hypothesized roles in chloride affinities and/or transport kinetics, as determined by structure-function studies and mutation studies using other halorhodopsins. Spectral residues were targeted based on their predicted retinal flanking locations based on crystal structures, and/or have been shown previously to govern the spectrum of bacteriorhodopsin. Alignments to H. salinarum halorhodopsin and bacteriorhodopsins were made using NCBI Blast.

TABLE 3

| | Homo- | Homo- | | | |
|---|---|---|---|---|---|
| | logous | logous | Primary | Primary Peak ± | |
| Halo | H. | H. | predicted | FWHM | Recovery |
| point | salinarum | salinarum | outcome | (nm), | of active |
| mu- | HR | BR | of muta- | second order | pumping |
| tant | residue | residue | tion | Gaussian fit | in dark? |
| Wild | N/A | N/A | N/A | 584 ± 51 | No |
| type | | | | | |
| T126H | T111 | D85 | Recovery | No measured | No |
| | | | | photocurrent | |
| T126R | T111 | D85 | Recovery | No measured | No |
| | | | | photocurrent | |
| W127F | W112 | W86 | Spectral | No measured | No |
| | | | shift | photocurrent | |
| S130T | S130 | T89 | Recovery | 568 ± 55 | No |
| S130D | S130 | T89 | Recovery | No measured | No |
| | | | | photocurrent | |
| S130H | S130 | T89 | Recovery | No measured | No |
| | | | | photocurrent | |
| S130R | S130 | T89 | Recovery | No measured | No |
| | | | | photocurrent | |
| A137T | A122 | D96 | Recovery | 585 ± 52 | No |
| A137D | A122 | D96 | Recovery | 575 ± 53 | No |
| A137H | A122 | D96 | Recovery | No measured | No |
| | | | | photocurrent | |
| A137R | A122 | D96 | Recovery | No measured | No |
| | | | | photocurrent | |
| G163C | G148 | G122 | Spectral | No measured | No |
| | | | shift | photocurrent | |
| W179F | W164 | W137 | Spectral | No measured | No |
| | | | shift | photocurrent | |
| S183C | F168 | S141 | Spectral | No measured | No |
| | | | shift | photocurrent | |
| F187M | F172 | M145 | Spectral | 589 ± 52 | No |
| | | | shift | | |
| F187A | F172 | M145 | Spectral | No measured | No |
| | | | shift | photocurrent | |
| K215H | R200 | R149 | Recovery | 586 ± 50 | No |
| K215R | R200 | R149 | Recovery | 575 ± 51 | No |
| K215Q | R200 | R149 | Recovery | 585 ± 56 | No |
| T218S | T203 | T178 | Recovery | 582 ± 53 | No |
| T218D | T203 | T178 | Recovery | No measured | No |
| | | | | photocurrent | |
| T218H | T203 | T178 | Recovery | No measured | No |
| | | | | photocurrent | |
| T218R | T203 | T178 | Recovery | No measured | No |
| | | | | photocurrent | |
| W222F | W207 | W182 | Spectral | No measured | No |
| | | | shift | photocurrent | |
| P226V | P211 | P186 | Spectral | No measured | No |
| | | | shift | photocurrent | |
| P226G | P211 | P186 | Spectral | No measured | No |
| | | | shift | photocurrent | |
| W229F | W214 | W189 | Spectral | 587 ± 53 | No |
| | | | shift | | |

Describing the methods employed in brief: codon-optimized genes were synthesized by Genscript and fused to GFP in lentiviral and mammalian expression vectors as used previously for transfection or viral infection of neurons. Primary hippocampal or cortical neurons were cultured and then transfected with plasmids or infected with viruses encoding for genes of interest, as described previously. Images were taken using a Zeiss LSM 510 confocal microscope. Patch clamp recordings were made using glass microelectrodes and a Multiclamp 700B/Digidata electrophysiology setup, using appropriate pipette and bath solutions for the experimental goal at hand. Neural pH imaging was done using carboxy-SNARF-1-AM ester (Invitrogen). Cell health was assayed using Trypan blue staining (Gibco). HEK cells were cultured and patch clamped using standard protocols. Mutagenesis was performed using the QuikChange kit (Stratagene). Computational modelling of light propagation was done with Monte Carlo simulation with MATLAB. In vivo recordings were made on headfixed awake mice, which were surgically injected with lentivirus, and implanted with a headplate as described before. Glass pipettes attached to laser-coupled optical fibers were inserted into the brain, to record neural activity during laser illumination in a photoelectrochemical artifact-free way. Data analysis was performed using Clampfit, Excel, Origin, and MATLAB. Histology was performed using transcardial formaldehyde perfusion followed by sectioning and subsequent confocal imaging.

A method of quantitative analysis of opsin-GFP membrane expression was used: The cell contour was first enhanced using the blur and subtraction methodology. A Magic Wand tool was used to define the pixels corresponding to the cell membrane. However, the tool sometimes selected the whole somatic cytoplasm and the processes, because some neuronal processes were too small to be separated into membrane vs. cytosol, causing the appearance of connectedness, and/or because the well-defined membrane processes overlap with other neurons or extend to the edge of the image. Line sections were generated at the apparent boundary of the soma and its processes, to separate sub-resolution image components from the soma (drawn as red here). The Magic Wand tool could now select distinct membrane segments of the soma. Membrane expression was then quantified by taking the area-weighted average of membrane pixel values, in the original image.

Using this method, it was found that the absolute expression level of Arch on the plasma membrane was similar to that of Halo (p>0.2, N=5 cells each). Experimenting with adding targeting sequences that improve trafficking showed that adding a signal sequence from the MHC Class I antigen (‘ss’) as well as the Kir2.1 ER export sequence (‘ER2’) (which is part of the sequence that boosts Halo currents by 75%, resulting in eNpHR), did not augment Arch currents (p>0.6, N=16 Arch cells, N=9 ss-Arch-ER2 cells). Thus, the effect of a given trafficking-improving sequence on opsin expression is opsin-specific (and perhaps species-specific), but nonetheless deserves further attention. For example, adding the Prolactin signal sequence (‘Prl’) to the N terminus of Arch trended towards boosting the Arch current (+32%; p<0.08, N=18 ss-Prl-Arch cells). Improving opsin targeting, however, is unlikely to alter opsin recovery kinetics or light dynamic range.

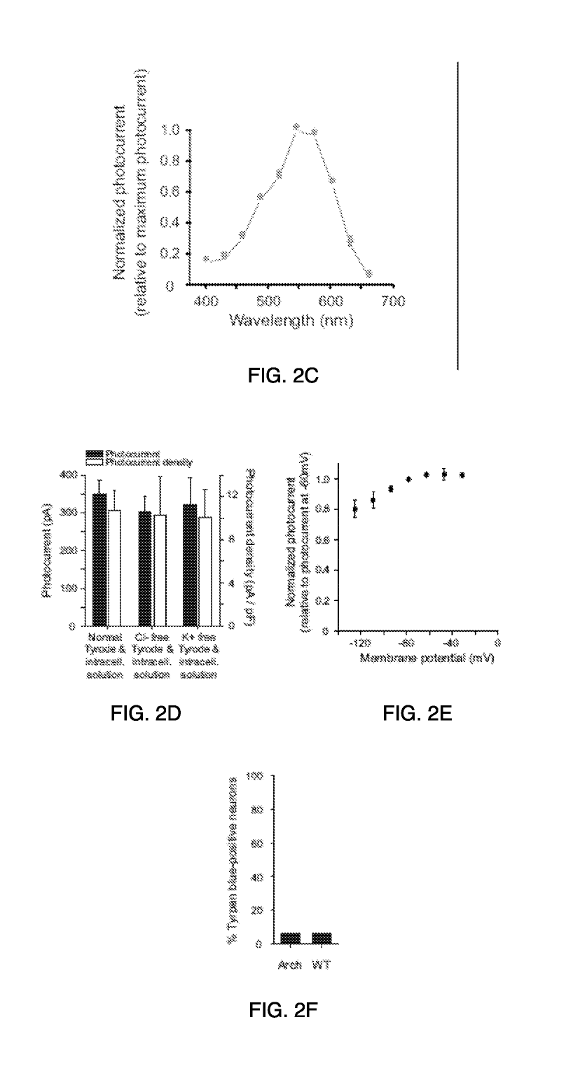

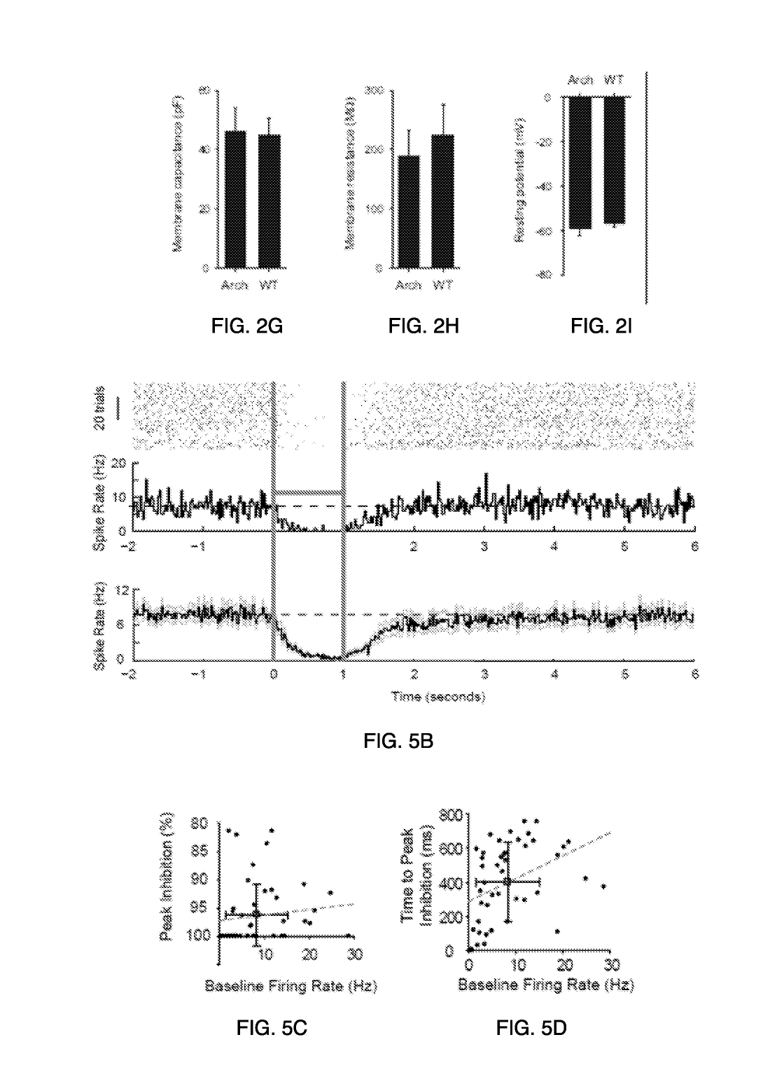

FIGS. 2A-I present functional properties of a light-driven proton pump Arch in neurons. FIGS. 2A and 2B are graphs of the photocurrents of Arch versus Halo measured as a function of 575±25 nm light irradiance in patch-clamped cultured neurons (n=4-16 neurons for each point), for low (FIG. 2A) and high (FIG. 2B) light powers. As seen in FIGS. 2A and 2B, Arch produces between a 2.7× and 16× more photocurrent than the current state-of-the-art gene product (N. pharaonis halorhodopsin, also denoted as “NpHR” or “Halo”) at 6 mW/mm² yellow light illumination and 0.04 mW/mm², respectively (wavelength=575±25 nm). FIG. 2C is an action spectrum of Arch measured in cultured neurons by scanning illumination light wavelength through the visible spectrum (N=7 neurons).

FIG. 2D depicts a photocurrent of Arch measured as a function of ionic composition (575±25 nm light, 7.8 mW/mm²), showing no significant dependence of photocurrent on concentration of Cl− or K+ ions (N=16, 8 and 7 neurons, from left to right). FIG. 2E depicts Arch proton photocurrent vs. holding potential (N=4 neurons). Together, FIGS. 2D and 2E demonstrate conclusively that Arch functions as a proton pump.

FIG. 2E is a histogram of Trypan blue staining of neurons lentivirally-infected with Arch vs. wild-type (WT) neurons, measured at 18 days in vitro (N=669 Arch-expressing, 512 wild-type, neurons). FIGS. 2G-I are histograms of membrane capacitance (FIG. 2G), membrane resistance (FIG. 2H), and resting potential (FIG. 21) in neurons lentivirally-infected with Arch vs. wild-type (WT) neurons, measured at 11 days in vitro (N=7 cells each). Together, FIGS. 2F-I demonstrate that expression of Arch does not harm or alter the physiology of the neurons in which it is expressed.

The magnitude of Arch-mediated photocurrents was large. At low light irradiances of 0.35 and 1.28 mW/mm², neural Arch currents were 120 and 189 pA respectively; at higher light powers (e.g., at which Halo currents saturate), Arch currents continued to increase, approaching 900 pA at effective irradiances of 36 mW/mm², well within the reach of typical in vivo experiments. The high dynamic range of Arch may enable excellent utilization of light sources (e.g., LEDs, lasers) that are safe and effective for optical control in vivo.

Several lines of evidence supported the idea that Arch functioned as an outward proton pump when expressed in neurons. Removing the endogenous ions that commonly subserve neural inhibition, Cl⁻ and K⁺, from physiological solutions did not alter photocurrent magnitude (p>0.4 comparing either K⁺ free or Cl⁻ free solutions to regular solutions, t-test). In solutions lacking Na⁺, K⁺, Cl⁻, and Ca²⁺, photocurrents were still no different from those measured in normal solutions (p>0.4; N=4 neurons tested without these four charge carriers). The reversal potential appeared to be less than −120 mV, also consistent with Arch being a proton pump.

The voltage swings driven by illumination of current-clamped Arch-expressing cultured neurons were assessed. As effective irradiance increased from 7.8 mW/mm² to 36.3 mW/mm², voltage clamped neurons exhibited peak currents that increased from 350±35 pA (N=16 neurons) to 863±62 pA (N=8 neurons) respectively. Current-clamped neurons under these two irradiance conditions were hyperpolarized by −69.6±7.3 mV (N=10) and −76.2±10.1 mV (N=8) respectively. Surprisingly, these voltage deflections, while both large, were not significantly different from one another (p>0.7, t-test), suggesting the existence of a rapidly activated transporter or exchanger (perhaps the Na⁺-dependent Cl⁻/HCO₃⁻ exchanger), or the opening of hyperpolarization-gated channels capable of shunting protons, which limit the effects of Arch on accumulated proton (or other charge carrier) gradients across neural membranes. This enabling of effective but not excessive silencing may make Arch safer than pumps that accumulate ions without self-regulation.

The changes in intracellular pH (pHᵢ) driven by illumination of Arch-expressing cultured neurons was assessed, using the fluorescent pH indicator carboxy-SNARF-1. Within one second of illumination with strong green light, pHᵢ rose from 7.309±0.011 to 7.431±0.020, plateauing rapidly. pHᵢ increased slightly further after 15 of illumination, to 7.461±0.024. The fast stabilization of pHᵢ may reflect the same self-limiting influence that limits proton-mediated voltage swings as described above, and may contribute to the safe operation of Arch in neurons by preventing large pHᵢ swings. The changes in pHᵢ observed here are comparable in magnitude to those observed during illumination of ChR2-expressing cells (due to the proton currents carried by ChR2) and are also within the magnitudes of changes observed during normal neural activity. Passive electrical properties of neurons were not affected by Arch expression (p>0.6 for each measure, t-test), nor was cell death (p>0.6, χ²=0.26).

The tissue volumes that could be silenced were estimated, using in vitro experiments and computational modeling. In cultured neurons expressing Arch or a trafficking-improved variant of Halo, eNpHR, brief current pulses were somatically injected at magnitudes chosen to mimic the current drives of neurons in the intact nervous system. These neurons were exposed to periods of 575 nm yellow light (0.35, 1.28, or 6 mW/mm², simulating irradiance ˜1.7, 1.2, or 0.6 mm away from the tip of a 200 micron fiber emitting 200 mW/mm² irradiance, as modelled by Monte Carlo methods, and measured the reduction in spike rate for each condition. In general, Arch-expressing neurons were significantly more inhibited than eNpHR-expressing cells. According to the model and the 350 pA data, the increase in brain tissue volume that would be 45-55% optically silenced would be ˜10× larger for Arch than for eNpHR.

One aspect of the invention is the use of the proton pumping rhodopsin genes from eukaryotes, such as algae, such as Acetabularia acetabulum, or fungus, such as Leptosphaeria maculans, P. tritici-repentis, and S. sclerotorium. These fungal rhodopsins are efficacious light-activated proton pumps.

In illustrative implementations, leptosphaeria maculans rhodopsin hyperpolarizes cells in strong response to blue light with sufficient spectral independence from the majority of electrogenic hyperpolarizing microbial rhodopsins that absorb more red-shifted light to enable the use of multiple colors of light to hyperpolarize different sets of cells in the same tissue by expressing pumps with different activation spectra genetically in different cells and then illuminating the tissue with different colors of light. For example, if one set of cells in a tissue (for example, but not limited to, excitatory neurons) express Halo, and a second set express Arch, then illuminating the tissue with 630 nm light will preferentially hyperpolarize the first set, whereas illuminating the tissue with 470 nm light will preferentially hyperpolarize the second set. Other pairs of targets that can be modulated with two colors of light in the same illumination area include, but are not limited to, two projections to/from one site, or combinations of the cell, its projections, and its organelles, given the ability to target the molecule sub-cellularly.

The light-driven proton pump Mac had an action spectrum strongly blueshifted relative to that of the light-driven chloride pump Halo. It was found that Mac-expressing neurons could undergo 4.1-fold larger hyperpolarizations with blue light than with red light, and Halo-expressing neurons could undergo 3.3-fold larger hyperpolarizations with red light than with blue light, when illuminated with appropriate filters. Accordingly, selective silencing of spike firing in Mac-expressing neurons in response to blue light and selective silencing of spike firing in Halo-expressing neurons in response to red light were demonstrated. Thus, the spectral diversity of proton pumps points the way towards independent multi-color silencing of separate neural populations. For example, two neuron classes, or two sets of neural projections from a single site, can be independently silenced during a behavioral task.

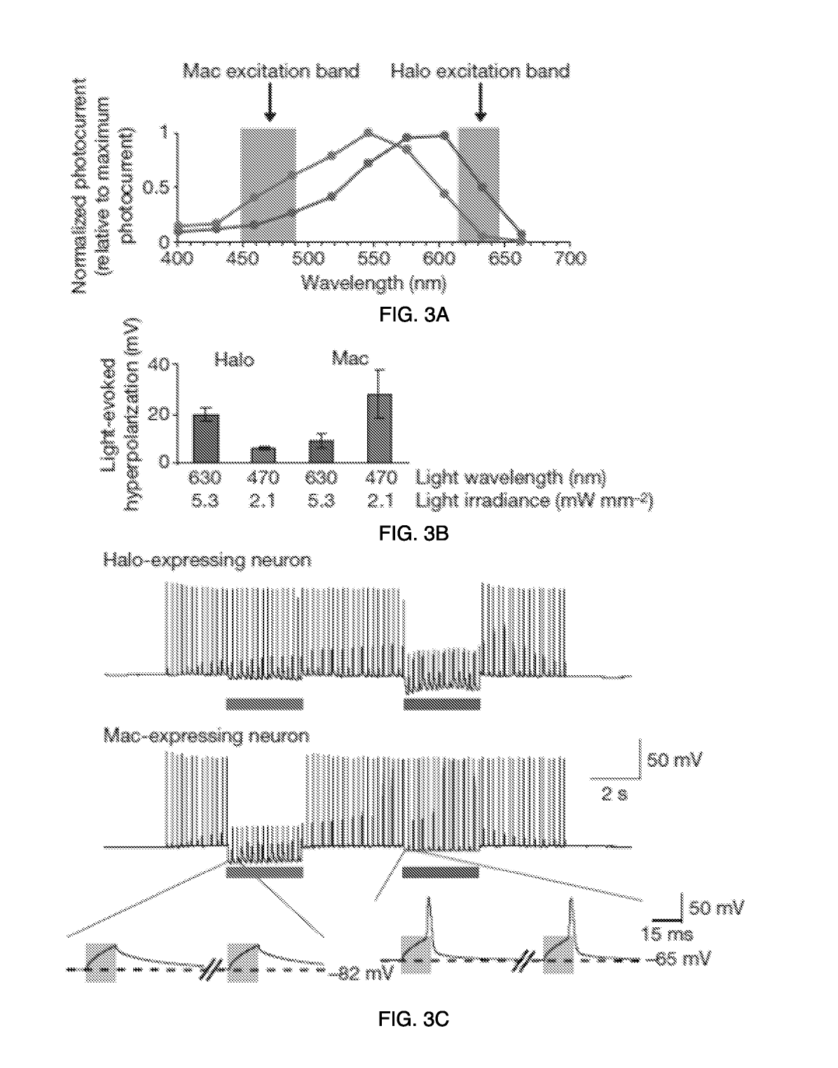

FIGS. 3A-C depict multicolor silencing of two neural populations, enabled by blue- and red-light drivable ion pumps of different classes. FIG. 3A is a graph depicting the action spectra of Mac versus Halo. In FIG. 3A, rectangles indicate filter bandwidths used for multicolour silencing in vitro. Blue light is delivered by a 470±20 nm filter at 5.3 mW/mm², and red light is delivered by a 630±15 nm filter at 2.1 mW/mm². FIG. 3B is a graph depicting membrane hyperpolarizations elicited by blue versus red light, in cells expressing Halo or Mac (n=5 Mac-expressing and n=6 Haloexpressing neurons). FIG. 3C graphically depicts that action potentials evoked by current injection into patch-clamped cultured neurons transfected with Halo (top) were selectively silenced by the red light but not by the blue light, and vice-versa in neurons expressing Mac (middle). Grey boxes in the inset (bottom) indicate periods of patch-clamp current injection.

In an illustrative implementation, the following methodology is used. First, take a Halobacterium sodomense gene for archaerhodopsin, human codon-optimized DNA, or a gene for leptosphaeria maculans rhodopsin, mammalian codon-optimized DNA, and express it in cells. In an illustrative implementation, the gene is expressed in cells according to the methodology that follows.

To start, clone the opsin gene into a lentiviral or adeno-associated virus (AAV) packaging plasmid, or another desired expression plasmid, and then clone GFP downstream of the gene, eliminating the stop codon of the opsin gene, thus creating a fusion protein. The viral or expression plasmid may contain either a strong general promoter, a cell-specific promoter, or a strong general promoter followed by one or more logical elements (such as a lox-stop-lox sequence, which will be removed by Cre recombinase selectively expressed in cells in a transgenic animal, or in a second virus, thus enabling the strong general promoter to then drive the gene).

If using a viral plasmid, synthesize the viral vector using the viral plasmid, using standard techniques. If using a virus, inject the virus using a small needle or cannula into the area of interest, thus delivering the gene encoding the opsin fusion protein into the cells of interest. If using another expression vector, directly electroporate or inject that vector into the cell or organism (for acutely expressing the opsin, or making a cell line, or a transgenic mouse or other animal).

Illuminate with light. For Arch, peak illumination wavelengths are 566 nm±66 nm (when incident intensity is defined in photons/second; second order Gaussian fit maximum±full width at half-maximum). For Mac, peak illumination wavelengths are 550 nm±69 nm. To illuminate two different populations of cells (e.g., in a single tissue) with two different colors of light, first target one population with Halo, and the other population with Mac, using two different viruses (e.g., with different coat proteins or promoters) or two different plasmids (e.g., with two different promoters). Then, after the molecule expresses, illuminate the tissue with 450±25 nm, 475±25 nm, or 500±25 nm light to preferentially hyperpolarize the Mac-expressing cells, and illuminate the tissue with 625±25 nm light, 600±25 nm light, or 575±25 nm light, to preferentially hyperpolarize the Halo-expressing cells. The above wavelengths illustrate typical modes of operation, but are not meant to constrain the protocols that can be used, and it will be clear to one of skill in the art of the invention that many other protocols are suitable for use in the present invention For example, either narrower or broader wavelengths, or differently-centered illumination spectra, can be used. For prosthetic uses, the devices used to deliver light may be implanted. For drug screening, a xenon lamp or LED can be used to deliver the light.

An experimental implementation of the invention demonstrated high-performance Arch-mediated optical neural silencing of neocortical regions in awake mice. Fluorescence images of a neuron in the awake mouse brain, expressing Arch, and silenced in response to yellow light, were recorded with a glass micropipette, thus demonstrating in vivo use of this molecule. Cells were healthy and tolerated the expression of the molecule well. After light illumination ceased, the neuron easily restored to its original, natural spike rate. This demonstrates the creation of temporary lesions of neural information content.

To directly assess Arch in vivo, lentivirus encoding for Arch was injected into mouse cortex and neural responses were recorded ˜1 month later. Arch expressed well and appeared well localized to the plasma membrane, labeling cell bodies, processes, and dendritic spines. Neurons in awake headfixed mice were recorded, illuminating neurons via a 200 micron optical fiber coupled to a 593 nm laser (power at electrode tip estimated at ˜3 mW/mm²). Upon light onset, firing rates of many units immediately and strongly declined, and remained low throughout the period of illumination, for both brief and long pulses. 13 single units were recorded that showed any decrease in firing during illumination, and spiking rates during exposure to 5 s yellow light were found to drop by an average of 90±15% (mean±standard deviation (SD)), restoring to levels indistinguishable from baseline after light cessation (p>0.2, paired t-test). 6 of the 13 units decreased spike rate by at least 99.5%, and the median decrease was 97.1%. One possibility is that Arch-expressing cells were almost completely silenced, whereas non-infected cells decreased activity due to network activity reduction during illumination; note that only excitatory cells were genetically targeted here. Optical silencing was consistent across trials (p>0.1, paired t-test comparing, for each neuron, responses to first 3 vs. last 3 light exposures; ˜20 trials per neuron). The kinetics of silencing were rapid: for the 6 neurons that underwent >99.5% silencing, spike firing reduced with near-0 ms latency, rarely firing spikes after light onset; averaged across all cells, firing rate reductions plateaued within 229±310 ms after light onset (mean±SD). After light cessation, firing rate restored quickly for the highly-silenced neurons; averaged across all cells, firing rates took 355±505 ms to recover after light offset. The level of post-light firing did not vary with repeated light exposure (p>0.7, paired t-test comparing, for each neuron, after-light firing rates during first 3 vs. the last 3 trials). Thus, Arch could mediate reliable, near-digital silencing of neurons in the awake mammalian brain.

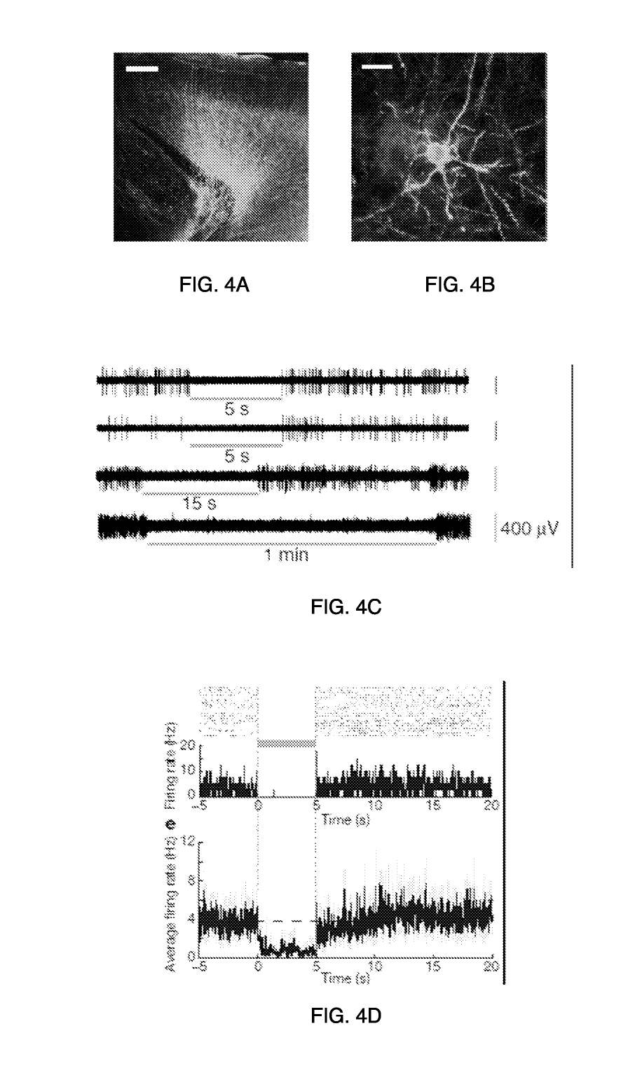

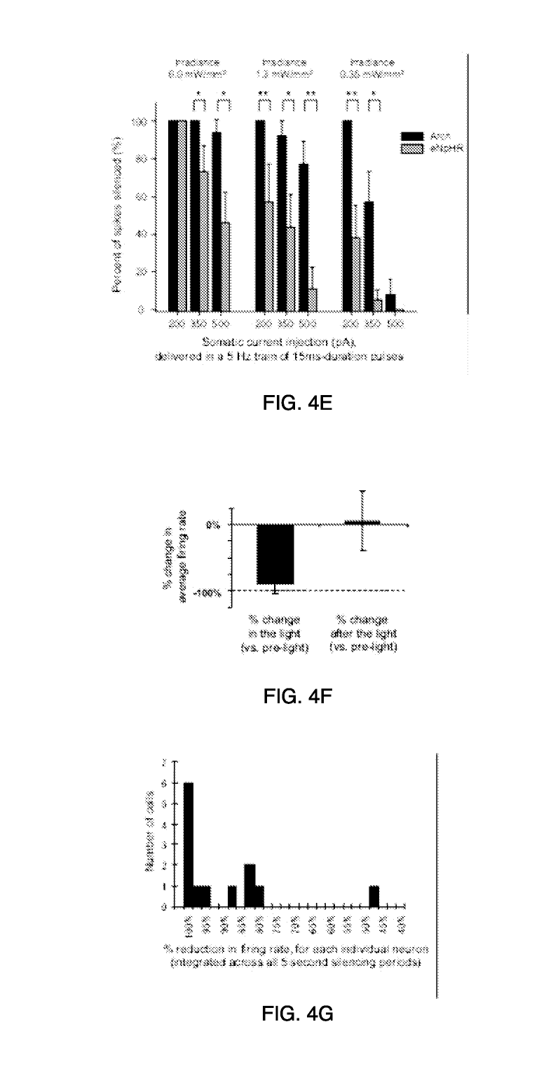

FIGS. 4A-G depict results from this experimental implementation of the invention, using high-performance Arch-mediated optical neural silencing of neocortical regions in awake mice. FIGS. 4A and 4B are fluorescence images showing Arch-GFP expression in the mouse cortex, 1 month after lentiviral injection. Scale bars, 200 um (left) and 20 um (right). FIG. 4C presents four representative extracellular recordings showing neurons undergoing 5-s, 15-s and 1-min periods of light illumination (593 nm; 150 mW/mm² radiant flux out the fibre tip; and expected to be 3 mW/mm² at the electrode tip, 800 mm away, based on Monte Carlos modeling). In FIG. 4D, neural activity in a representative neuron before, during, and after 5-s of yellow light illumination, is shown as a spike raster plot and as a histogram of instantaneous firing rate averaged across trials (bottom; bin size, 20 ms). Population average of instantaneous firing rate before, during and after yellow light illumination (black line, mean; gray lines, mean±SE; n=13 units).

FIG. 4E presents in vitro data showing, in cultured neurons expressing Arch or eNpHR and receiving trains of somatic current injections (15 ms pulse durations at 5 Hz), the percent reduction of spiking under varying light powers (575±25 nm light) as might be encountered in vivo. *, p<0.05; **, p<0.01, t-test. N=7-8 cells for each condition, demonstrating that Arch performs better with statistical significance when compared to the state of the art. FIG. 4F is a histogram depicting average change in spike firing during 5 seconds of yellow light illumination (left) and during the 5 seconds immediately after light offset (right), for the data shown in FIG. 4D, demonstrating that illumination and silencing does not alter the excitability of the neuron. FIG. 4G is a histogram of percentage reductions in spike rate, demonstrating the high success rate using an illustrative implementation of the present invention.

Another aspect of the invention is the reduction to practice of temporally precise, reversible, safe, and cell type-specific silencing of the awake primate brain, here shown in the macaque parietal cortex, silenced by Arch under 532 nm illumination. Such silencing has been demonstrated with both Arch and ArchT.

FIG. 5A depicts the temporally precise, reversible, repeatable, and cell type-specific silencing of the non-human primate via Arch (here, shown for the macaque parietal cortex). As seen in FIG. 5A, firing activity of a multi-unit, recorded in the Macaque parietal cortex was dramatically reduced upon exposure to 1 second green light (532 nm laser, blue dash) 2 months after injection of high titer lentivirus carrying Arch-GFP gene behind the 1.3 kb CaMKII promoter. (Top) Spike raster shows 20 trials. (Bottom) Histogram of instantaneous firing rate across all trials, bin=5 ms.

FIG. 5B depicts the temporally precise, reversible, repeatable, and cell type-specific silencing of the non-human primate via ArchT (here, shown for the macaque parietal cortex). As seen in FIG. 5B, upon green light illumination (532 nm), primate cortical neurons expressing ArchT decrease their firing rate (sample individual neuron, top) and achieved near 100% silencing after a few hundred milliseconds. On average, of the 46 single units, neurons reached peak silencing of 96.3%±5.5%, mean±standard deviation, n=46 neurons) 402±233 ms after light onset. 24 of the 46 neurons were 100% silenced (population average, middle). The amount of silencing achieved upon illuminating ArchT expressing neurons is independent of their baseline firing rates (linear regression, p=0.3, R̂2=0.022) (FIG. 5C). However, it trends to take longer to silence a neuron with higher baseline firing rate (linear regression p=0.007, R̂2=0.153) (FIG. 5D).

This demonstrates pre-clinical translational viability and technology viability, the latter as evident by the ability to silence large volumes of brain tissue (estimated to be larger than the primary volume of tissue that the virus infects) with far more numerous and active depolarizing synaptic inputs to overcome than in the anaesthetized or awake rodent.

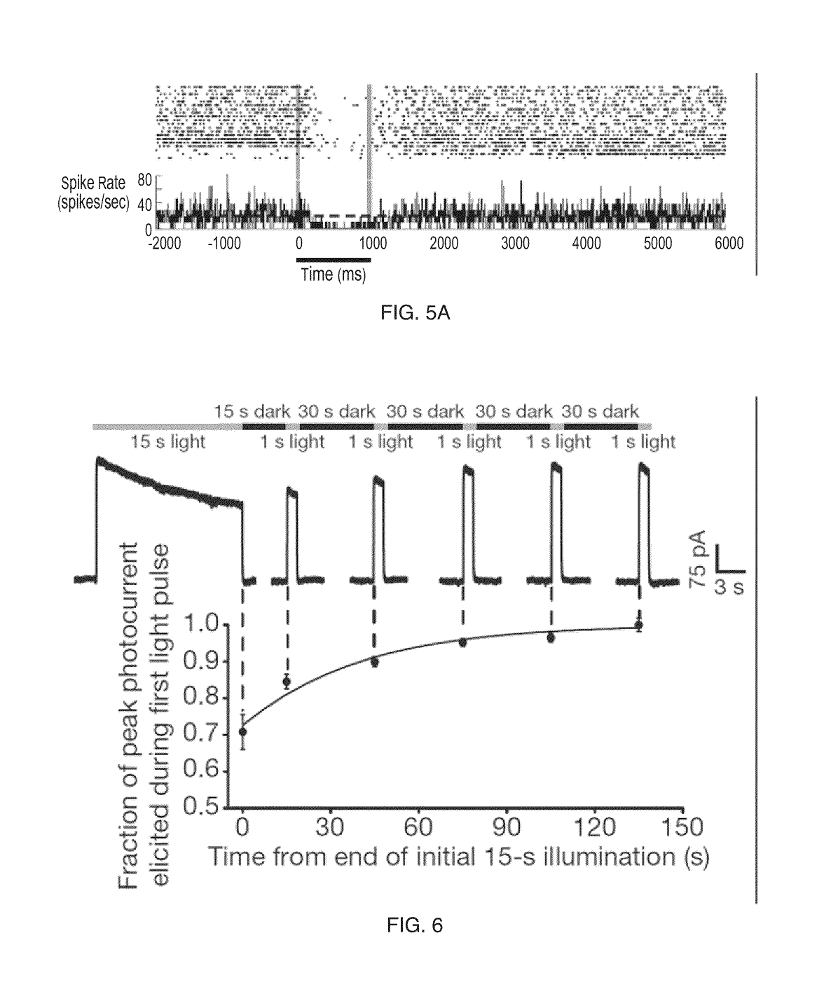

Experimental implementation of the invention has demonstrated that, while Halo may require blue light to recover it to its original state after prolonged yellow light illumination, Arch does not—after prolonged illumination, it recovers spontaneously in the dark. This key advantage is essential for prosthetics or other biotechnology applications in which multiple colors of light are neither optimal nor desired. For example, the ability to quickly repeat the silencing protocol with repeatable molecule efficacy enhances the capability to perform within-subject correlations. Shown in FIG. 6 are raw current trace of a neuron lentivirally infected with Arch, illuminated by a 15-s light pulse (575±25 nm, irradiance 7.8 mW/mm²) followed by 1-s test pulses delivered at 15, 45, 75, 105 and 135 s after the end of the 15-s light pulse, and population data of averaged Arch photocurrents (n=11 neurons) sampled at the times indicated by the vertical dotted lines that extend into the top trace.

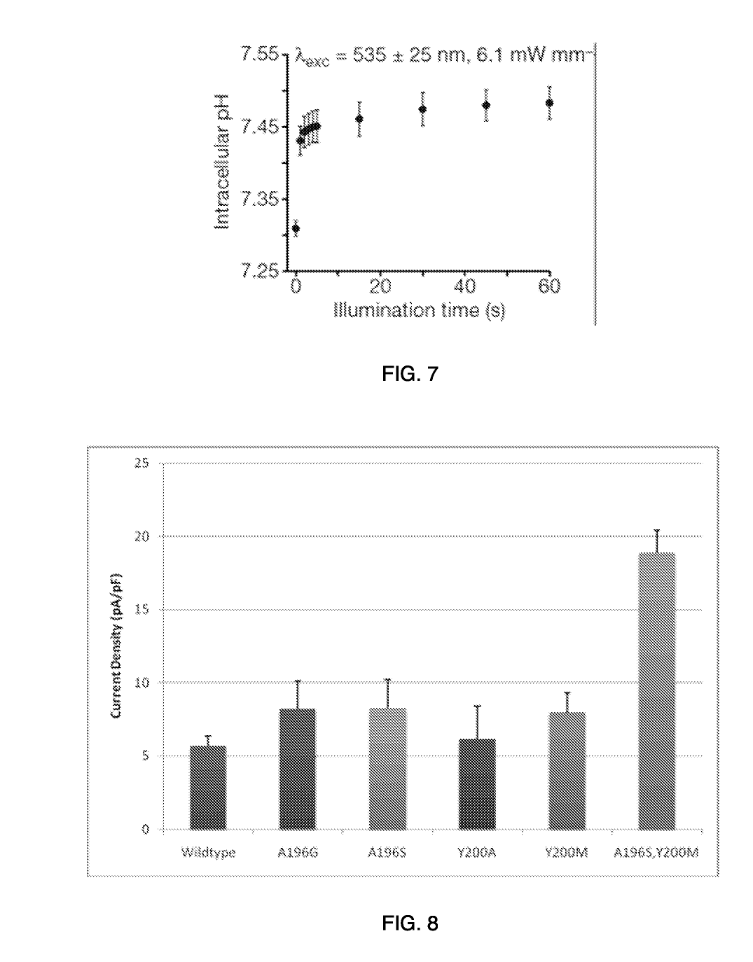

Proton pumps were also employed according to one aspect of the present invention to alter intracellular pH using light. Shown in FIG. 7 are intracellular pH measurements in neurons expressing Arch over a 1-min period of continuous illumination and simultaneous imaging (535±25 nm light, 6.1 mW/mm²) using SNARF-1 pH-sensitive ratiometric dye (n=10-20 cells per data point). This result also demonstrates that silencing neural activity via light-driven proton pumps leads to controllable pH changes that are on the order of normal neural activity as a measure of safety. The simultaneous alteration of pH and membrane potential can be utilized for a combined therapeutic effect, such as simultaneous treatment of cortical spreading depression and its accompanying acidification that are commonly observed in migraine, stroke, and ischemia.

Another aspect of the invention includes enhancements to the functional performance of the heterologously expressed proton pumps in mammalian cells via site-directed mutagenesis. The performance of these example compositions of matter may be altered by site-directed mutagenesis, such as the A196S+Y200M double mutation to Mac that leads to 3.3-fold improvement in photocurrent density. The performance of the above may also be altered by appending N-terminal and C-terminal peptide sequences to affect cellular trafficking

FIG. 8 is a graph showing peak current density recorded from Mac mutants using whole-cell patch clamp that enhance photocurrent generation, where “wild type” denotes Mac (leptosphaeria maculans rhodopsin). As shown in FIG. 8, mutants were expressed in HEK293FT cells and illuminated with 575±25 nm light at 7.8 mW/mm² irradiance. The A196S+Y200M double mutant had a 3.3-fold increase in photocurrent than wild type Mac, under the stated conditions.

One aspect of the present invention is the creation of a class of bi-directional control molecules, exemplified by the W96Y-like mutants of archaerhodopsins. Such single molecules can be used as shunt-like molecules, where the inhibitory photocurrent is limited by the voltage dependence of the molecule's multiple modes of ion translocation for more naturalistic and controlled neural silencing (e.g. may avoid triggering hyperpolarization-dependent depolarizing currents).

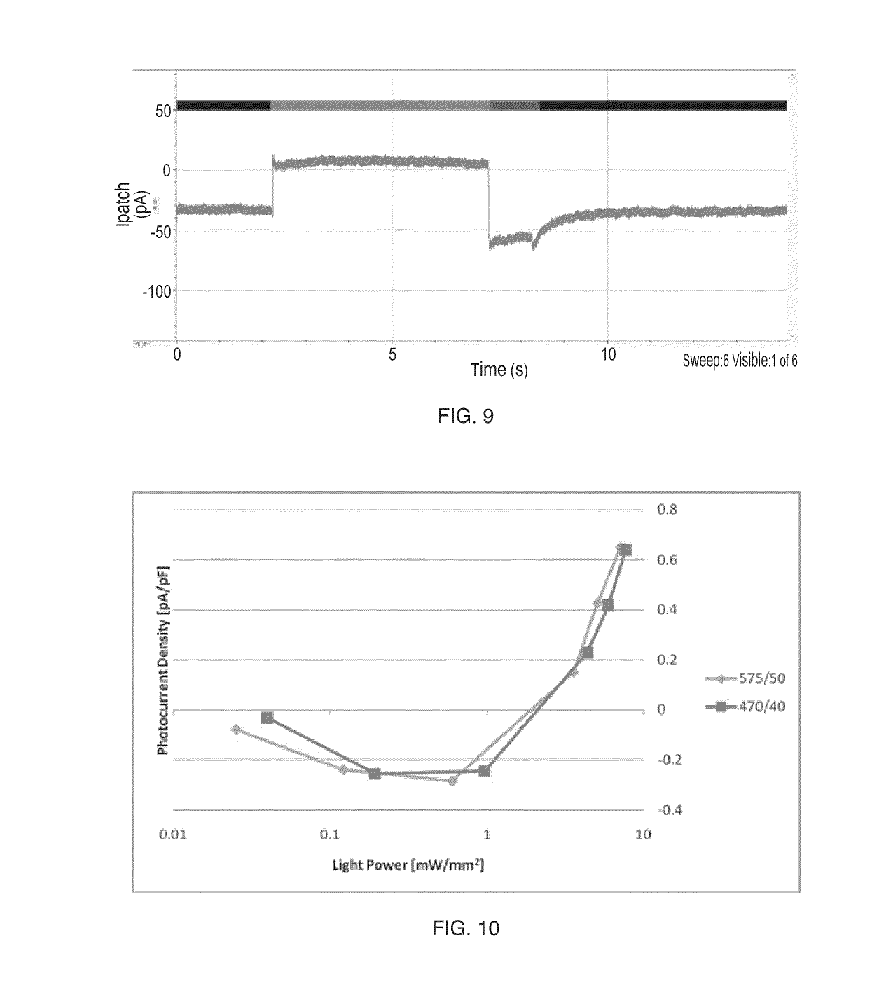

In this aspect of the present invention, bi-directional proton pumps were engineered from archaerhodopsins, created by the analogous W96Y mutation of H. salinarum bacteriorhodopsin (from here on, this analogous position and mutation will be known as “W96Y-like”. FIG. 9 is a raw current trace recorded in a HEK cell expressing ArchT(w), illuminated with orange light (orange dash, 607±36 nm), followed by near-ultraviolet light (purple dash, 436±20 nm), thus demonstrating the potentiality for bi-directional control of membrane voltage using two different colors of light to address one molecule.

The direction of proton translocation via these molecules can be affected by color, intensity of illumination, or a combination of the two. The corresponding mutant of ArchT will be denoted as ArchT(w). Other archaerhodopsins from H. sodomense, Halorubrum strain aus-1 and Halorubrum strain aus-2 also resulted in this bi-directional functionality, whereas mutations to other microbial rhodopsins such as the H. salinarum bacteriorhodopsin, Mac, and channelrhodopsin, respectively, did not lead to such bi-directional molecules, and thus this engineered functionality is non-obvious to one of ordinary skill in the art.

FIG. 10 is a plot depicting bi-directional optical control of an archaerhodopsin “W96Y-like” variant derived from Halorubrum strain aus-2, similar to the ArchT(w) variant of Halorubrum strain TP009. Here the direction of ion-translocation is determined by illumination intensity regardless of wavelength (here shown for 470±20 nm and 575±25 nm illumination). In FIG. 10, light power is plotted on a logarithmic scale.

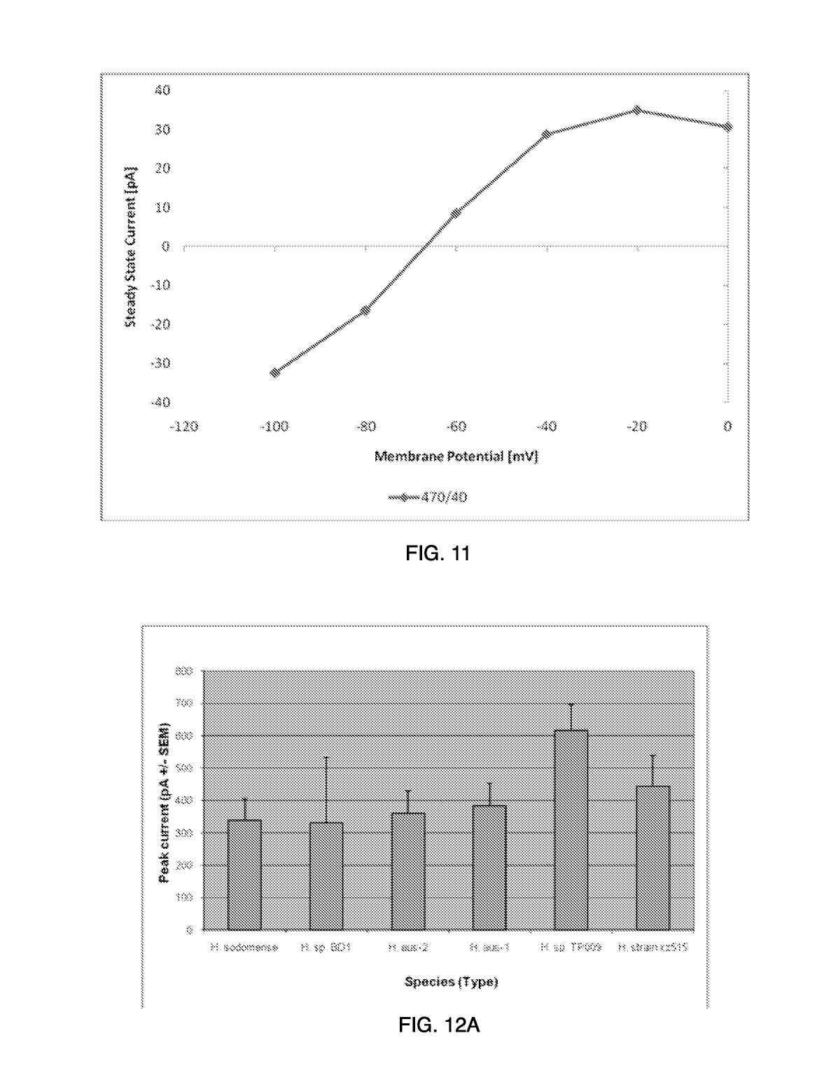

FIG. 11 is a plot depicting optically induced shunt-like activity exhibited by an archaerhodopsin “W96Y-like” variant derived from Halorubrum strain aus-1, similar to the ArchT(w) variant of Halorubrum strain TP009. When a HEK cell is illuminated with 470±20 nm light at 8 mW/mm², the reversal potential is between −65 and −70 mV.

Related to the W96Y-like mutants of archaerhodopsins is the variable control of membrane conductance, with respect to optical illumination wavelength, intensity, and transmembrane voltage. Unlike naturally occurring bi-directional molecules that function in mammalian cells, such as fSR's, these engineered variants have sustained steady state currents. The other known bi-directional molecules to date, proteorhodopsins, do not function under mammalian physiological conditions, as assessed in HEK cells and mouse primary neuron culture with the proteorhodopsins commonly known as BAC31A8, Hot75m4, and PalE6

In another aspect of the present invention, proton pumps are targeted to organelles in multi-cellular systems, multi-cellular organisms, and mammalian cell lines. In an exemplary implementation, ArchT was successfully targeted to mitochondria of mammalian HEK cell lines via the appendage of the human cytochrome c oxidase VIII N-terminal mitochondrial targeting sequence. The ability to augment proton gradients across the mitochondrial membrane with light enhances ATP production similar to as performed at the whole single-cell organism level with E. coli; the conversion of light to metabolizable forms of energy may decrease the innate dependence of the cell on glucose-dependent energy production and oxidative phosphorylation that can generate harmful reactive oxygen species and oxidative stress. Additional examples of mitochondrial targeting sequences include, but are not limited to TOM70, NADH ubiquinone oxoreductase, aldehyde dehydrogenase-2, ATP synthase alpha-subunit, and mitochondrial ATP-ase inhibitor.

One aspect of the invention uses light-activated proton pumps to hyperpolarize neurons. Light-activated microbial proton pumps have significantly improved kinetics over their chloride counterparts (e.g., N. pharaonis halorhodopsin) because they lack the long-lived inactive states, and in the case of proton-pumping archaerhodopsins versus halorhodopsins from various classes of species (bacteria, halobacter, haloarcula, and halorubrum chloride pumps), are demonstrably faster This enables more temporally precise silencing, as well as more consistent long-term silencing for neural prosthetics and treatments of disease. Many disorders are disorders of neural excitability; the ability to shut down specific kinds of cells would greatly enhance the treatment of them.

Leptosphaeria maculans rhodopsin is blue-activatable, and thus allows hyperpolarization of cells with a color of light heretofore not used in biotechnology for hyperpolarization of cells. By using Mac and Halo together, hyperpolarization of two different populations of cells in the same tissue or in the same culture dish becomes possible. This simultaneous, two-color inactivation, is particularly promising for complex tissues such as the brain.

Other functional molecules were used in mammalian cells during the screening process, including but not limited to: cruxhalorhodopsins (chloride pumps from Haloarcula), archaerhodopsins and cruxrhodopsins (proton pumps from Halorubrum and Haloarcula, respectively) as efficacious silencers of neural activity that traffic well to mammalian membranes, and Acetabularia acetabulum as an exemplar of algal rhodopsins, which are blue-shifted from proton pumps from the archaeal kingdom.

The performance of these molecules can be tuned for optimal use, particularly in context of their use in conjunction with other molecules or optical apparatus. For example, in order to achieve optimal contrast for multiple-color silencing, one may desire to either improve or decrease the performance of one molecule with respect to one another, by the appendage of trafficking enhancing sequences or creation of genetic variants by site-directed mutagenesis, directed evolution, gene shuffling, or altering codon usage. Molecules or classes of molecules may have inherently varying spectral sensitivity that may be functionally advantageous in vivo (where scattering and absorption will vary with respect to wavelength, coherence, and polarization), by tuning the linearity or non-linearity of response to optical illumination with respect to time, power, and illumination history.

One particular aspect of the invention is the use of the halorubrum genus of haloarchaea. These have been identified as particularly efficacious light-activated proton pumps because they express particularly well in mammalian membranes and perform robustly under mammalian physiological conditions, based on the shown characterization of every full archaerhodopsin clone known at the time of initial disclosure.

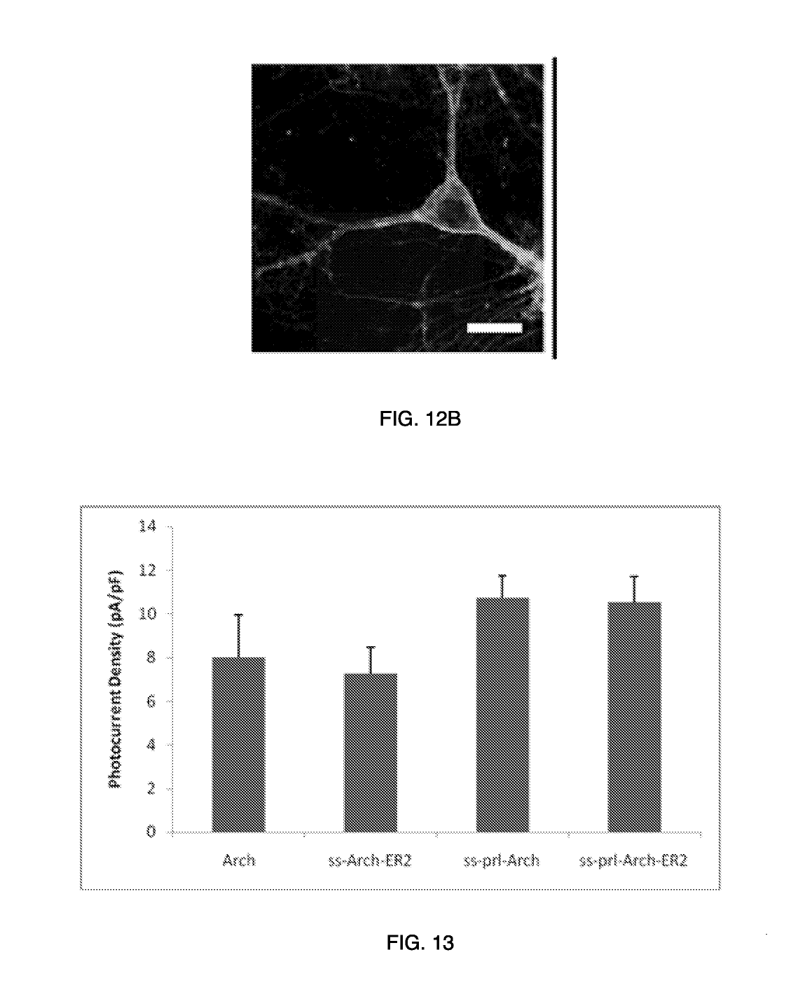

FIG. 12A is a histogram of photocurrents measured in (mouse hippocampus) cultured neurons expressing all known electrogenic archaerhodopsin full sequence clones (at the time of disclosure), demonstrating that the class of proton pumps from halorubrum perform exceptionally well under mammalian physiological conditions and produce photocurrents >2.5× greater than the state of the art naturally occurring gene product (at the time of the disclosure). FIG. 12B is a confocal fluorescence image of cultured neuron expressing Arch with a GFP fused to the C-terminus (scale bar=20 um) showing good membrane localization in the absence of the appended signal sequences (i.e. the naturally occurring gene product or protein sequence). It should be noted that H. lacusprofundiopsin was shown to be non-electrogenic in its primary physiological role, bearing semblance to fSR's, and thus it is not shown.

Another aspect of the present invention is the enablement of systematic tuning of membrane trafficking properties by appending, deleting, or replacing, small peptide sequences. For example, the prolactin (PRL) signal sequence boosts Arch photocurrents by ˜34%, while the ER2 sequence (C-terminal ER export sequence from KiR2.1) reduces intracellular blebbing but does not increase the photocurrent; the effects of the two sequences can therefore be systematically combined for an trafficking- and photo-current improved variant, hereby termed sp-Arch-ER2, as an example in the form of signal sequence::PRL::“product”::ER2, where “product” is a genetically encoded gene product.

FIG. 13 is a histogram depicting the cumulative effect of appending signal sequences to a naturally occurring protein sequence. As shown in FIG. 13, intracellular blebs or puncta are reduced by the appendage of an N-terminal “ss” and the C-terminal “ER2” sequence as has been previously reported, but do not enhance the photocurrent density. The addition of the “ss” and “PRL” sequences to the N-terminus improves the current density by 34%. The Arch variant bearing all 3 moieties has reduced intracellular agglomeration of protein (conferred by the ss+ER2 combination) and improved photocurrent density like the ss-PRL-Arch variant.

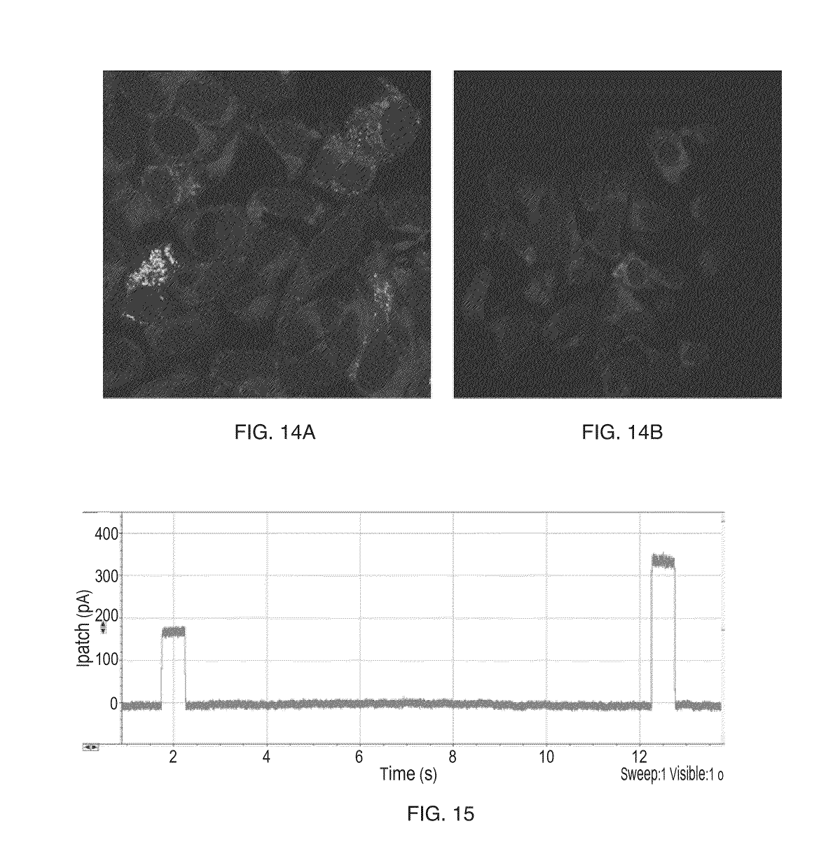

In illustrative implementations, proton pumps may be targeted to specific organelles. For example, proton pumps targeted to the mitochondria can augment or diminish the ATP-production capability of the cell, and thus affect cell metabolism. In illustrative implementations, proton pumps may be targeted to organelles to affect the physiology of multi-cellular organisms. In an exemplary implementation of this application, FIGS. 14A and B are fluorescence images showing HEK293 cells cultured on coverslips that were transfected with MTS8-GFP (FIG. 14A) and MTS8-ArchT-GFP (FIG. 14B) plasmids. Cultures were then stained with Mitotraker Red CMXRos (Invitrogen) on post-transfection day 2 and fixed in 4% paraformaldehyde before being mounted on a glass slide for confocal imaging.

An aspect of the invention is the use of the proton pumping rhodopsin genes from eukaryotes, such as algae or fungus. Besides Mac, another fungal rhodopsin that has been identified as being a particularly efficacious light-activated proton pump is S. sclerotorium, a fungal opsin that has never been characterized before physiologically. FIG. 15 is a trace of the S. sclerotorium opsin in a HEK cell demonstrating that it too is efficacious. As shown in FIG. 15, a voltage clamped HEK293 cell expressing the S. sclerotorium proton pump exhibits photocurrents upon illumination with blue (470 nm) and yellow light (575 nm).

Another aspect of the invention is the use of inwardly rectifying proton pumps. For example, a molecule like a transducer-free sensory rhodopsin exhibits bi-directional proton transport at two different wavelengths, and thus can be used to depolarize or hyperpolarize a cell using the same protein, as well as treat acidosis or alkalinosis with the same protein.

Another aspect of the invention is the generation of locally acidic or alkaline environment proximal to the light-activated proton pump, thereby affecting the binding of membrane receptors to their targets (for example, acid-sensing ion channels) by modulating pH. Such a method may be used to alter the efficacy of pH dependent pharmacological agents, such as the treatment of cancer where the tumor environment is highly acidic, or for the deprotection of pharmacological agents bearing acid- or base-labile protecting groups.

In one embodiment of the invention, sensitizing chromophores, such as chlorophyll or salinixanthin, are used to broaden or shift the absorbance spectrum of the molecule, and are particularly advantageous for multi-color silencing, tuning the absorbance for optimality with specific optical apparatus (e.g. narrow excitation LEDs and lasers, long wavelength absorption for better transmission through tissue, etc.), or the creation of harmful UV oxidized species.

Another aspect of the invention is the identification of the analogous amino acid resides to the retinal flanking S141, M145, and P186 residues in H. salinarum bacteriorhodopsin as mutagenesis targets for systematic spectral tuning Mutagenesis to these residues in ArchT and Mac more easily resulted in altered spectral responses at higher and lower energies of the peak absorption (by boosting or reducing the higher energy or lower energy responses, independently or in concert) often without decrease in physiological function, when compared to other retinal flanking positions identified with previously reported molecules. For example, the S151G mutant of ArchT (analogous to S141 residue above mentioned) blue-shifted the peak absorption of the wild type molecule by 18 nm, and the A196+Y200M double mutant of Mac (corresponding to the S141+M145 residues mentioned above) red-shifted the wild-type Mac's peak absorption by 21 nm.

Another aspect of the invention is the reduction to practice of technologically viable deep brain silencers or deep brain inhibitors (hereby termed DBSi or DBI, respectively) in the primate brain. Deep brain silencing may be used in conjunction with, deep brain stimulation, for example, to limit adverse side effects created by electrical stimulation that affects all cell types.

Another aspect of the invention is the method of coupling the molecule's efficacy to the selection pressure or screening criteria in directed evolution. For example, if the molecule is screened in an archaebacteria devoid of a microbial rhodopsin that it utilizes for phototrophy, then species bearing poorly performing molecules under the test conditions will not survive. Such coupling of performance to selection enables autonomous and label-free screening.

Another aspect of the invention are various compositions of matter that have been reduced to practice, including plasmids encoding for the above genes, especially lentiviruses carrying payloads encoding for the above genes, adeno-associated viruses carrying payloads encoding for the above genes, cells expressing the above genes, animals expressing those genes (including mice, but implicating primates and humans).

In one application of the invention, cells are hyperpolarized, thus activating endogenous hyperpolarization-activated conductances (such as T-type calcium channels and Ih currents), and then drugs are applied that modulate the response of the cell to hyperpolarization (using a calcium or voltage-sensitive dye). This enables new kinds of drug screening using just light to activate the channels of interest, and using just light to read out the effects of a drug on the channels of interest.

Another application of the invention is the use of light-activated proton pump to increase the pH of the cell. Such a technique may be used to treat acidosis, particularly as a method of neuroprotection during traumatic brain injury.

In yet another application, light-activated proton pumps are employed for the coupled effects of hyperpolarization and intracellular alkalinization. For example, both phenomena can induce spontaneous spiking in neurons by triggering hyperpolarization-induced cation currents or pH-dependent hyper-excitability.

Other applications of the invention include, but are not limited to, use of proton pumps of microbial origin as an exemplary test system for assessing membrane protein trafficking and physiological function in heterologously expressed systems, generation of sub-cellular voltage or pH gradients, particularly at synapses and in synaptic vesicles to alter synaptic transmission, and mitochondria to improve ATP synthesis, and use of light-activated proton pumps to hyperpolarize a cell that has high intracellular chloride concentrations, such as young adult-born neurons.

In illustrative implementatations, this invention may be used for one or more of the following: The use of light activated proton pumps to adjust the voltage potential of cells, sub-cellular regions, and extracellular regions. The use of light activated proton pumps to adjust the pH of cells, sub-cellular regions, and extracellular regions. The use of light activated proton pumps to release protons as chemical transmitters. These methods wherein the proton pump is a microbial rhodopsin that is outwardly rectifying, is from the halorubrum genus of archaeabacteria, is leptosphaeria maculans, P. triticirepentis, or S. scelorotorium, or is from the genus Acetabularia, and specifically halorubum strain aus-1, halorubrum strain aus-2, halorubrum sodomense, halorubrum strain BD1, halorubrum stain xz515, halorubrum strain TP009, halorubrum lacusprofundi, or Acetabularia acetabulum, leptosphaeria maculans, pyrenophora tritici-repentis, or sclerotinia sclerotorium.

These methods may be used for neural silencing, that is, hyperpolarizing a neuron to prevent it from depolarizing, spiking, or otherwise signaling (e.g., to release neurotransmitters). The use of two or more light-activated membrane proteins for multi-color neural silencing (e.g., Ace or Mac, and Halo), expressing them in different populations of cells in a tissue or in a dish, and the illuminating them each with different colors of light. The use of light-activated proton pumps instead of chloride channels to improve the regeneration speed of the active pumping state of hyperpolarizing electrogenic membrane proteins. The method wherein the proton pump is a microbial rhodopsin that in itself is both inwardly and outwardly rectifying at two different colors of light. The use of light-activated proton pumps to treat cellular and muscle acidosis or alkalinosis. The use of light-activated proton pumps to treat acidosis and alkalinosis in the cardiac system. The use of light-activated proton pumps for neuroprotection after traumatic brain injury. The use of light-activated proton pumps to modify cellular pH, which then modifies cellular potential, or vice versa. The use of light-activated proton pumps to generate pH and electrogenic gradients in vesicles to mediate their transport, fusion with membranes, or incorporation of molecules. The use of light-activated proton pumps to treat pain or irritation induced by molecular interactions with acid- or alkaline-sensitive ion channels or receptors. The use of light-activated proton pumps to hyperpolarize cells that have high intracellular chloride content. The use of light-activated proton pumps to modify pH for the purpose of affecting cellular, multi-cellular, or organismal development.

The ability to optically perturb, modify, or control cellular function offers many advantages over physical manipulation mechanisms, such as speed, non-invasiveness, and the ability to easily span vast spatial scales from the nanoscale to macroscale. One such approach is an opto-genetic approach, in which heterologously expressed light-activated membrane proteins move ions with spectral selectivity, as well as potential ion-selectivity and cell-type specificity, the latter by way of promoter-targeting. The light-activated cation channels channelrhodopsin-2 (ChR2) and volvox channelrhodopsin-1 (VChR1) have been demonstrated to depolarize neurons with millisecond resolution (i.e. the timescale of an action potential) by primarily triggering sodium influx into the cell. Likewise, the light-activated chloride pump NpHR has been used to silence neural activity by hyperpolarizing cells by way of chloride influx. The light-activated G-protein coupled receptor RO4 has also been used to silence neural activity by way of decreasing pre-synaptic calcium conductance.

In illustrative implementations, this invention may be used to enable large currents, fast inhibition, control involving H+ ions, not Cl− ions; multi-color control of cells, and the ability to change pH of cells. Furthermore, with Mac, multiple-color silencing of multiple populations of cells in the same tissue or in the same culture dish is enabled. Mac-expressing cells are hyperpolarizable using blue light, whereas Halo-expressing cells are hyperpolarizable using yellow light; the two cells can sit side-by-side, and be individually addressed with different colors of light.

Detailed methods. Novel opsin reagents: plasmid construction and lentivirus production. The opsins examined are listed in Table 1, which describes their molecule classes, species of origin, GenBank Accession numbers, and relevant references. Molecule classes chiefly include bacteriorhodopsins (proton pumps) and halorhodopsins (chloride pumps). Further sub-classifications of ion pump type denote the origin of the species: for example, a “cruxhalorhodopsin” is a chloride pump from the haloarcula genus of halobacteria. Opsins were mammalian codon-optimized, and were synthesized by Genscript (Genscript Corp., NJ). Opsins were fused in frame, without stop codons, ahead of GFP (using BamHI and Agel) in a lentiviral vector containing the CaMKII promoter, enabling direct neuron transfection, HEK cell transfection (expression in HEK cells is enabled by a ubiquitous promoter upstream of the lentiviral cassette), and lentivirus production (except for Halobacterium salinarum halorhodopsin, which was fused to GFP in the vector pEGFP-N3 (using EcoRI and BamHI) and only tested by transfection). eNpHR was synthesized by inserting the signaling sequence from the acetycholine receptor beta subunit.

Replication-incompetent VSVg-pseudotyped lentivirus was produced, which allows the preparation of clean, non-toxic, high-titer virus (roughly estimated at ˜10⁹-10¹⁰ infectious units/mL). Briefly, HEK293FT cells (Invitrogen) were transfected with the lentiviral plasmid, the viral helper plasmid pΔ8.74, and the pseudotyping plasmid pMD2.G. The supernatant of transfected HEK cells containing virus was then collected 48 hours after transfection, purified, and then pelletted through ultracentrifugation. Lentivirus pellet was resuspended in phosphate buffered saline (PBS) and stored at −80° C. until further usage in vitro or in vivo.

Hippocampal and cortical neuron culture preparation, transfection, infection, and imaging. All procedures involving animals were in accordance with the National Institutes of Health Guide for the care and use of laboratory animals and approved by the Massachusetts Institute of Technology Animal Care and Use Committee. Swiss Webster or C57 mice (Taconic or Jackson Labs) were used. For hippocampal cultures, hippocampal regions of postnatal day 0 or day 1 mice were isolated and digested with trypsin (1 mg/ml) for ˜12 min, and then treated with Hanks solution supplemented with 10-20% fetal bovine serum and trypsin inhibitor (Sigma). Tissue was then mechanically dissociated with Pasteur pipettes, and centrifuged at 1000 rpm at 4° C. for 10 min. Dissociated neurons were plated at a density of approximately four hippocampi per 20 glass coverslips, coated with Matrigel (BD Biosciences). For cortical cultures, dissociated mouse cortical neurons (postnatal day 0 or 1) were prepared as previously described, and plated at a density of 100-200 k per glass coverslip coated with Matrigel (BD Biosciences). Cultures were maintained in Neurobasal Medium supplemented with B27 (Invitrogen) and glutamine. Hippocampal and cortical cultures were used interchangeably; no differences in reagent performance were noted.

Neurons were transfected at 3-5 days in vitro using calcium phosphate (Invitrogen). GFP fluorescence was used to identify successfully transfected neurons. Alternatively, neurons were infected with 0.1-1 μl of lentivirus per well at 3-5 days in vitro. Throughout the paper, neurons were transfected unless indicated as having been infected. All images and electrophysiological recordings were made on neurons 9-14 days in vitro (approximately 6-10 days after transfection or viral infection).

Confocal images of infected neurons in culture (briefly fixed in 4% paraformaldehyde) were obtained with a Zeiss LSM 510 confocal microscope (63× magnification objective lens). Culture images were maximum intensity projections made from sets of 5 images (1.0 μm image plane thickness) spaced along the z-axis by 0.5 micron steps. Quantitative confocal analysis of membrane expression of opsins was performed using infected neurons in culture (10 days post-infection, briefly fixed in 4% paraformaldehyde). Images were obtained with a Zeiss LSM 510 confocal microscope (63× magnification objective lens), always with the same illumination and observation parameters to avoid procedural variability. Given the near-100% viral infection rate, isolated neurons were chosen for analysis in order to reduce background fluorescence from nearby neurons and their processes. Images were analyzed in ImageJ (National Institutes of Health), based on a neuron-adapted version of a previously reported algorithm used to assay membrane expression of channelrhodopsins and channelrhodopsin variants in HEK cells. An image was first filtered with a 2-pixel Gaussian blur, and the filtered image was subtracted from the original one to enhance the contour of the cell. These steps are exactly the same as those utilized before in HEK cells. However, because the membranes of neurons do not form simple shapes like the HEK cells as the original algorithm was designed for, black line sections were applied to separate the neuronal cell body from the processes. The magic wand can then accurately select the somatic membrane segments of a neuron, which can then be analyzed by the pixel intensity-value extraction method described for HEK cells. The value of the membrane fluorescence for a given neuron, reported in the text, was then defined as the area-weighted average of the line segments. While this method cannot prove that a given patch of membrane-proximal fluorescence exists exclusively on the outermost membrane (in principle a patch of fluorescence could reside just under the membrane), it does serve to discriminate between surface expression and ER retention, and has previously been validated in predicting functional physiological surface expression.

In vitro patch clamp recording and optical methods. Whole cell patch clamp recordings were made on neurons at 9-14 days in vitro, using a Multiclamp 700B amplifier, Digidata 1440 digitizer, and a PC running pClamp (Molecular Devices). During recording, neurons were bathed in Tyrode solution containing (in mM): 125 NaCl, 2 KCl, 3 CaCl₂, 1 MgCl₂, 10 HEPES, 30 glucose, 0.01 NBQX, and 0.01 gabazine, at pH 7.3 (NaOH adjusted), and with 305-310 mOsm (sucrose adjusted). Borosilicate glass (Warner) pipettes were filled with a solution containing (in mM): 125 K-Gluconate, 8 NaCl, 0.1 CaCl₂, 0.6 MgCl₂, 1 EGTA, 10 HEPES, 4 Mg-ATP, 0.4 Na-GTP, at pH 7.3 (KOH adjusted), and with 295-300 mOsm (sucrose adjusted). Pipette resistance was 5-10 MΩ; access resistance was 10-30 MΩ, monitored throughout the voltage-clamp recording; resting membrane potential was ˜−60 mV in current-clamp recording.

For ion selectivity tests, neurons were bathed in chloride-free recording solution containing (in mM): 125 Na-Gluconate, 2 K-Gluconate, 3 CaSO₄, 1 MgSO₄, 10 HEPES, 30 glucose, 0.01 NBQX, 0.01 gabazine, at pH 7.3 (NaOH adjusted), and with 305-310 mOsm (sucrose adjusted), or potassium-free recording solution containing (in mM): 125 NaCl, 2 CsCl, 3 CaCl₂, 1 MgCl₂, 10 HEPES, 30 glucose, 0.01 NBQX, 0.01 gabazine, at pH 7.3 (NaOH adjusted), 305-310 mOsm (sucrose adjusted). During these ion selectivity tests, pipettes were filled with chloride-free pipette solution containing (in mM): 125 K-Gluconate, 8 Na-Gluconate, 0.1 CaSO₄, 0.6 MgSO₄, 1 EGTA, 10 HEPES, 4 Mg-ATP, 0.4 Na-GTP, pH 7.3 (KOH adjusted), 295-300 mOsm (sucrose adjusted), or potassium-free pipette solution containing (in mM): 125 Cs-methanesulfonate, 8 Na-Gluconate, 0.1 CaSO₄, 0.6 MgSO₄, 1 EGTA, 10 HEPES, 4 Mg-ATP, 0.4 Na-GTP, pH 7.3 (CsOH adjusted), 295-300 mOsm (sucrose adjusted). During resting membrane potential shifting, neurons were bathed in recording solution containing (in mM): 125 N-methyl-D-glucamine, 2 Cs-methanesulfonate, 3 CdSO₄, 1 MgSO₄, 10 HEPES, 30 glucose, 0.01 NBQX, 0.01 gabazine, pH 7.3 (H₂SO₄ adjusted), 305-310 mOsm (sucrose adjusted), and pipettes were also filled with analogous solutions containing (in mM): 125 Cs-methanesulfonate, 8 N-methyl-D-glucamine, 0.1 CdSO₄, 0.6 MgSO₄, 1 EGTA, 10 HEPES, 4 Mg-ATP, 0.4 Tris-GTP, pH 7.3 (CsOH adjusted), 295-300 mOsm (sucrose adjusted).

Photocurrents were measured with 1-second or 15-second duration light pulses in neurons voltage clamped at −60 mV. Light-induced membrane hyperpolarizations were measured with 1-second light pulses, in neurons current clamped at their resting membrane potential. For all experiments except for the action spectrum characterization experiments, a DG-4 optical switch with 300W xenon lamp (Sutter Instruments) was used to deliver light pulses. The DG-4 was controlled via TTL pulses generated through a Digidata signal generator. A 575±25 nm bandpass filter (Chroma) was used to deliver yellow light, and a 535±25 nm filter was used to deliver green light. For selective activation of Halo versus Mac at different wavelengths, a 470±20 nm bandpass filter (Chroma) was used to deliver blue light (0.92 mW/mm², through a 40× objective), and a 630±15 (Chroma) was used to deliver red light (2.6 mW/mm²). For action spectrum characterization (Table 2), a Till Photonics PolyChrome V, 150 W Xenon, 15 nm monochromator bandwidth, was used.

Data was analyzed using Clampfit (Molecular Devices) and MATLAB (Mathworks, Inc.). Statistical analysis and curve fitting was done with Statview (SAS Institute), MATLAB, or Origin (OriginLab). Reported action spectra are second-order Gaussian fits (performed in MATLAB), because action spectra were asymmetric, with a broad “shoulder” at wavelengths shorter than the primary peak wavelength.

For the initial screening of photocurrents, yellow light (575±25 nm, 7.8 mW/mm², through a 40× lens) was chiefly used (see below for exceptions); accordingly, in order to adjust the screen data to reflect the photocurrent for each molecule that would have been observed at its respective spectral maximum, photocurrents were spectrum normalized by calculating the overlap integral between the second-order Gaussian fit of the action spectrum for each molecule and the passband of the yellow illumination filter used for the screen (or in other words, integrating the Gaussian fit between 550 and 600 nm), and then dividing that value by the integral of the whole action spectrum for that molecule. These resultant ratios, or “Spectral Screen Normalization Factors,” are summarized (normalized to that ratio for Halo itself) in the rightmost column of Table 2.

In the cases of gPR, bPR, and the Leptosphaeria maculans (Mac, LR, Ops) and Acetabularia acetabulum (Ace, AR) proton pump opsins, green light (535±25 nm, 9.4 mW/mm², through a 40× lens) was used during the screen, due to the significantly blue-shifted action spectrum of these genes. These four spectra were also normalized to the respective spectral maxima of each molecule, as described above, as well as weighted by the output power of the lamp. All screen photocurrents and spectra were measured in neurons except for the action spectrum of Ace, which was recorded in HEK 293FT cells for better resolution (due to the extremely small currents of Ace in neurons).

In order to extend the power characterization of Arch beyond the power of the yellow light available with the microscope and configuration that employed (7.8 mW/mm² irradiance, through a 40× lens), extrapolation to higher effective yellow powers was accomplished by equating various powers of unfiltered white light illumination from the DG4, to approximate effective yellow power equivalents. These effective irradiances were estimated by adjusting the output power of unfiltered white light from the DG4, and comparing the photocurrents vs. those generated with 575±25 nm yellow light in the same Arch-expressing neuron, at low light powers, until the photocurrent magnitudes were similar (p>0.7, paired t-test; N=6). In support of this method for estimating effective irradiances, no noticeable photocycle-accelerating effects of non-yellow light were observed for Arch. Light power-photocurrent curves, thus estimated, were fitted with a Hill plot. To compare to Arch, Halo currents measured for the dose response experiment were obtained using a Halo variant that demonstrated similar photocurrent densities compared to unmodified Halo (p>0.7, t-test; N=16), bearing a N-terminal signal sequence from a truncated MHC class I antigen and the C-terminal golgi export sequence from bovine rhodopsin; these measurements were then scaled by the photocurrent ratio between Halo and this variant measured at 7.8 mW/mm².

HEK cell culture, transfection, and electrophysiology. HEK 293FT cells (Invitrogen) were maintained in DMEM medium (Cellgro) supplemented with 10% fetal bovine serum (Invitrogen), 1% penicillin/streptomycin (Cellgro) and 1% sodium pyruvate (Biowhittaker)). For recording, cells were plated at 5-10% confluence on uncoated glass coverslips, where they adhered to surfaces typically within 12-18 hours. Adherent cells were transfected using TransIT 293 transfection kits (Minis). Cells were recorded by whole-cell patch clamp 1.5-2 days later, as described above for neurons, except that they were voltage clamped at −40 mV, with a Tyrode bath solution lacking GABAzine and NBQX.

Intracellular pH imaging. Intracellular pH (denoted pHᵢ) imaging was performed using a cell-permeant ratiometric fluorescent dye, carboxy-SNARF-1 AM ester (Invitrogen). In order to eliminate background fluorescence that would interfere with pHᵢ imaging using SNARF-1, the fusion protein comprising Arch and cyan fluorescent protein (CFP) was used. The DG-4 was used to deliver light pulses (6.1 mW/mm², through a 20× lens) via a green 535±25 nm bandpass filter (Chroma). Neurons were loaded with 10 μM SNARF-1-AM ester in Tyrode solution for 10 minutes, and then washed twice with Tyrode. Arch-expressing neurons were identified by their CFP fluorescence (Chroma CFP set, λ_{excitation}=436±20 nm, λ_{dicrhoic}=455 nm, λₑₘᵢₛₛᵢₒₙ=480±20 nm). After waiting 1 additional minute, Arch and the SNARF-1 dye were simultaneously excited with 535±25 nm light, and the dye was imaged near the isosbestic point of the dye for 500 ms using a 610±37.5 nm bandpass filter (λ_{dichroic}=565 nm, Chroma) to obtain a baseline SNARF loading level. After waiting another minute for the neuron to recover its initial pHᵢ, the neuron and dye were again excited with green light, and the dye was imaged at various time points with 1 second exposure lengths, using a 640±25 nm bandpass filter (λ_{chroic}=600 nm, Chroma). Arch-negative neighboring neurons in the same field of view were imaged to provide a basis for comparison, and also to provide baseline pH of the cells as a point of reference. Immediately after this period, the dye was again imaged near the isobestic point for 500 ms to assess for photo-bleaching or dye leakage; no change was observed (p>0.7 comparing before vs. after 60 seconds of illumination; n=5 neurons).

Calibration of the dye was performed by the “high K⁺/nigericin” method⁴², in which cells were immersed in a high K⁺ Tyrode-like solution containing (in mM): 125 KCl, 2 NaCl, 3 CaCl₂, 1 MgCl₂, 10 HEPES, 30 glucose, 0.01 NBQX, 0.01 gabazine, 0.001 TTX, pH 5.5 and pH 8.5 initial stock solutions (KOH adjusted), 305-310 mOsm (sucrose adjusted). The calibration curve was taken between pH 6.75 and 8 (N=51-110 neurons per calibration point; calibration curve linear region goodness of fit R²=0.999, between pH 7.15-8.0). Images were processed using ImageJ (National Institutes of Health).

Cell health assays. Membrane properties were measured on day 11 in vitro using algorithms built into pClamp10. Cell death was assayed at day 18 in vitro by incubating cultured neurons for 10 minutes in 0.04% Trypan Blue (Gibco) in Tyrode solution for 10 minutes at room temperature, washing with Tyrode solution, and then immediately counting the percentage of neurons stained. In order to limit variability across cell cultures and cell ages, Arch and wild type control neurons were chosen from the same cell cultures and tested on the same day.

Estimations of thresholds for silencing. Whole-cell current-clamped neurons were somatically injected with 5 Hz current pulse trains (15 ms pulse duration, 8 sec train duration) at 200, 350, or 500 pA, with or without yellow light (575 nm) illumination at irradiances of 6, 1.28, or 0.35 mW/mm² for 3 seconds, beginning 2 seconds into the current pulse train. Neurons that did not spike at all with 200 pA current pulses (15 ms pulse duration) were discarded. For all remaining cells, the probability of spiking in the dark, given a 200 pA/15 ms current input, was 84.0±10.5% and 82.8±3.5% for Halo- and Arch-expressing neurons, respectively (p>0.9, N=7-8 neurons each), and the probability of spiking in the dark was ˜100%, given ≧350 pA current inputs.

Virus injection. Under isoflurane anesthesia, 1 μL lentivirus was injected through a craniotomy made in the mouse skull, into the motor cortex (0.62 mm anterior, 0.5 mm lateral, and 0.5 mm deep, relative to bregma), or the sensory cortex (0.02 mm posterior, 3.2 mm lateral, and 2.2 mm deep, relative to bregma). Virus was injected at a rate of 0.1-0.2 μl/min through a cannula connected via polyethylene tubing to a Hamilton syringe, placed in a syringe pump (Harvard Apparatus). The syringe, tubing, and cannula were filled with silicone oil (Sigma). For mice used for in vivo recordings, custom-fabricated plastic headplates were affixed to the skull, and the craniotomy was protected with agar and dental acrylic.

In vivo physiology, optical neuromodulation, and data analysis. Recordings were made in the cortex of headfixed awake mice 1-2 months after virus injection, using glass microelectrodes of 5-20 MΩ impedance filled with PBS, containing silver/silver-chloride wire electrodes. Signals were amplified with a Multiclamp 700B amplifier and digitized with a Digidata 1440, using pClamp software (Molecular Devices). A 50 mW yellow laser (SDL-593-050T, Shanghai Dream Laser) was coupled to a 200 micron-diameter optical fiber in a fashion as described previously. The laser was controlled via TTL pulses generated through Digidata. Laser light power was measured with an 818-SL photodetector (Newport Co.). An optical fiber was attached to the recording glass electrode, with the tip of the fiber ˜600 μm laterally away from and ˜500 μm above the tip of the electrode (e.g., ˜800 microns from the tip), and guided into the brain with a Siskiyou manipulator at a slow rate of ˜1.5 μm/s to minimize deformation of the cortical surface.

Data was analyzed using MATLAB (Mathworks, Inc.). Spikes were detected and sorted offline using Wave_clus. Neurons suppressed during light were identified by performing a paired t-test, for each neuron, between the baseline firing rate during the 5 second period before light onset vs. during the period of 5 second light illumination, across all trials for that neuron, thresholding at the p<0.05 significance level. Instantaneous firing rate histograms were computed by averaging the instantaneous firing rate for each neuron, across all trials, with a histogram time bin of 20 ms duration. To determine the latency between light onset and the neural response, a 20 ms-long sliding window was swept through the electrophysiology data and the earliest 20 ms period that deviated from baseline firing rate was identified, as assessed by performing a paired t-test for the firing rate during each window vs. during the baseline period, across all trials for each neuron. Latency was defined as the time from light onset to the time at which firing rate was significantly different from baseline for the following 120 ms. The time for after-light suppression to recover back to baseline was calculated similarly.

Histology. Between 2 and 8 weeks after virus injection, mice were perfused through the left cardiac ventricle with ˜20 mL 4% paraformaldehyde in PBS (pH 7.3), and then the brain was removed and sectioned into 120-240 μm coronal sections on a vibratome in ice-cold PBS, and stored in PBS. Slices were mounted with Vectashield solution (Vector Labs), and visualized with a Zeiss LSM 510 confocal microscope using 20× and 63× objective lenses.

Monte Carlo modeling of light propagation. In MATLAB, a Monte Carlo simulation of light scattering and absorption in the brain from light emitted from the end of an optical fiber was performed by dividing a cube of gray matter into a 200×200×200 grid of voxels corresponding to 10 μm×10 μm×10 μm in dimension, using previously-published model parameters and algorithms. Data was interpolated from the literature to obtain a scattering coefficient for yellow (˜593 nm) light in brain gray matter of 13 mm⁻¹, and an absorption coefficient of 0.028 mm⁻¹. Since light propagation close to the optical fiber was of interest, before the orientation of photon trajectories is randomized by multiple scattering events, an anisotropic scattering model based upon the Henyey-Greenstein phase function was used, utilizing an anisotropy parameter of 0.89. 5×10⁶ packets of photons were launched in a fiberlike radiation pattern through a model fiber (Optran 0.48 HPCS, Thorlabs) with a numerical aperture of 0.48, and modeled their propagation into the brain based on an algorithm for Monte Carlo modeling of light transport in multi-layered tissues. In essence, whenever a photon packet entered a voxel, the program would probabilistically calculate the forecasted traveling distance before the next scattering event. If that traveling distance took the photon packet out of the starting voxel, then the packet would be partially absorbed appropriately for the distance it traveled within the starting voxel, and the process would then restart upon entry of the photon packet into the new voxel. If that traveling distance ended the trip of the photon packet within the starting voxel, then the packet would be absorbed appropriately for the distance it traveled within the starting voxel, and a new direction of packet propagation would be randomly chosen according to the Henyey-Greenstein function. Using this model, a graph was generated that shows the contours at which the light irradiance falls off to various percentages of the irradiance of light at the surface of the optical fiber, for yellow light. The model generally agrees with previous measurements, done in brain slices.

In one aspect, the present invention is a method for adjusting the voltage potential of cells, subcellular regions, or extracellular regions, comprising incorporating at least one gene encoding for a light-driven proton pump into at least one target cell, subcellular region, or extracellular region, the proton pump operating to change transmembrane potential in response to a specific wavelength of light, and causing the expression of the gene by exposing the target cell, subcellular region, or extracellular region to the specific wavelength of light in a manner designed to cause the voltage potential of the target cell, subcellular region, or extracellular region to increase or decrease. In an illustrative implementation, the proton pump may be a microbial rhodopsin that is outwardly rectifying, and in particular may be derived from the halorubrum genus of archaeabacteria or leptosphaeria maculans, P. triticirepentis, and S. scelorotorium. The voltage potential of the target cell, subcellular region, or extracellular region may be increased or decreased until it is hyperpolarized, particularly to achieve neural silencing. A plurality of light-activated proton pumps responsive to different wavelengths of light may be used to achieve multi-color neural silencing.

In another aspect, the present invention is a method for adjusting the pH of cells, subcellular regions, or extracellular regions, comprising incorporating at least one gene encoding for a light-driven proton pump into at least one target cell, subcellular region, or extracellular region, the proton pump operating to change cell, subcellular region, or extracellular region pH in response to a specific wavelength of light, and causing the expression of the gene by exposing the target cell, subcellular region, or extracellular region to the specific wavelength of light in a manner designed to cause the pH of the target cell, subcellular region, or extracellular region to increase or decrease. In an illustrative implementation, the proton pump may be a microbial rhodopsin that is outwardly rectifying, and in particular may be derived from the halorubrum genus of archaeabacteria or leptosphaeria maculans, P. triticirepentis, and S. scelorotorium.

In yet another aspect, the present invention is a method for causing cells, subcellular regions, or extracellular regions to release protons as chemical transmitters, comprising incorporating at least one gene encoding for a light-driven proton pump into at least one target cell, subcellular region, or extracellular region, the proton pump operating to cause proton release in response to a specific wavelength of light, and causing the expression of the gene by exposing the target cell, subcellular region, or extracellular region to the specific wavelength of light in a manner designed to cause the target cell, subcellular region, or extracellular region to release protons. In an illustrative implementation, the proton pump may be a microbial rhodopsin that is outwardly rectifying, and in particular may be derived from the halorubrum genus of archaeabacteria or leptosphaeria maculans, P. triticirepentis, and S. scelorotorium.

### Processors:

In exemplary implementations of this invention, one or more electronic processors are specially adapted: (1) to control the operation of, or interface with, hardware components of a system, including a wirelessly powered system for optical perturbation of optogenetically modified tissues, (2) to receive signals indicative of human input, (3) to output signals for controlling transducers for outputting information in human perceivable format, and (4) to process data, to perform computations, to execute any algorithm or software, and to control the read or write of data to and from memory devices. The one or more processors may be located in any position or positions within or outside of the wirelessly powered system. For example: (a) at least some of the one or more processors may be embedded within or housed together with other components of the wirelessly powered system, and (b) at least some of the one or more processors may be remote from other components of the wirelessly powered system. The one or more processors may be connected to each other or to other components in the wirelessly powered system either: (a) wirelessly, (b) by wired connection, or (c) by a combination of wired and wireless connections. For example, one or more electronic processors may be housed in a computer (e.g., a PC) or a microcontroller.

### DEFINITIONS

Here are a few definitions and clarifications.

The terms “a” and “an”, when modifying a noun, do not imply that only one of the noun exists.

To “activate” a neural target means to increase the electrical activity of the set of neurons that comprises the target. Normally neurons fire action potentials, and exhibit subthreshold activity as well; activating a target means to increase, perhaps to a high rate, the number of action potentials fired by a cell, or the electrical voltage of the cells, in the target. The target may be defined anatomically, functionally, molecularly, or otherwise (e.g., a set of neurons within a region, or distributed throughout the brain, that expresses a given molecule, or that is overactive in a given disease, or that responds to a given drug, or that projects from one region to another). To “inactivate” a neural target means to decrease the electrical activity of the set of neurons that comprises the neural target.

The term “comprise” (and grammatical variations thereof) shall be construed broadly, as if followed by “without limitation”. If A comprises B, then A includes B and may include other things.

“Defined Term” means a term that is set forth in quotation marks in this Definitions section.

For an event to occur “during” a time period, it is not necessary that the event occur throughout the entire time period. For example, an event that occurs during only a portion of a given time period occurs “during” the given time period.

The term “e.g.” means for example.

The fact that an “example” or multiple examples of something are given does not imply that they are the only instances of that thing. An example (or a group of examples) is merely a non-exhaustive and non-limiting illustration.

Unless the context clearly indicates otherwise: (1) a phrase that includes “a first” thing and “a second” thing does not imply an order of the two things (or that there are only two of the things); and (2) such a phrase is simply a way of identifying the two things, respectively, so that they each can be referred to later with specificity (e.g., by referring to “the first” thing and “the second” thing later). For example, unless the context clearly indicates otherwise, if an equation has a first term and a second term, then the equation may (or may not) have more than two terms, and the first term may occur before or after the second term in the equation. A phrase that includes a “third” thing, a “fourth” thing and so on shall be construed in like manner.

The term “fluid” shall be construed broadly, and includes gases and liquids.

The term “for instance” means for example.

“Herein” means in this document, including text, specification, claims, abstract, and drawings.

The terms “horizontal” and “vertical” shall be construed broadly. For example, “horizontal” and “vertical” may refer to two arbitrarily chosen coordinate axes in a Euclidian two dimensional space, regardless of whether the “vertical” axis is aligned with the orientation of the local gravitational field. For example, a “vertical” axis may oriented along a local surface normal of a physical object, regardless of the orientation of the local gravitational field.

The term “include” (and grammatical variations thereof) shall be construed broadly, as if followed by “without limitation”.

The term “light guide” includes a microfabricated wave guide for delivering light

The term “magnitude” means absolute value.

“Neural control technology” means one or more of the methods and apparatus (e.g., arrays of light fibers, arrays of microfabricated lightguides, and techniques of optical perturbation using optical neural control reagents) that are described in the Prior Applications.

To “optically silence” a neural target means to silence the neural target by optical perturbation.

To “optically activate” (or “optically stimulate”) a neural target means to activate the neural target by optical perturbation.

To “optically inactivate” (or “optically silence”) a neural target means to activate the neural target by optical perturbation.

The term “optical probe” includes an optical fiber, a waveguide, a probe that comprises multiple waveguides, or an array of any one or more of the above, including (a) an array of optical fibers or (b) an array of probes, which probes each comprise multiple waveguides.

The term “or” is inclusive, not exclusive. For example A or B is true if A is true, or B is true, or both A or B are true. Also, for example, a calculation of A or B means a calculation of A, or a calculation of B, or a calculation of A and B.

A parenthesis is simply to make text easier to read, by indicating a grouping of words. A parenthesis does not mean that the parenthetical material is optional or can be ignored.

As used herein, the term “set” does not include a so-called empty set (i.e., a set with no elements). Mentioning a first set and a second set does not, in and of itself, create any implication regarding whether or not the first and second sets overlap (that is, intersect).

To “silence” a neural target means to decrease the electrical activity of the set of neurons that comprises the target. Normally neurons fire action potentials, and exhibit subthreshold activity as well; silencing a target means to decrease, perhaps to zero, the number of action potentials fired by, or the electrical voltage of, the cells in the target. The target may be defined anatomically, functionally, molecularly, or otherwise (e.g., a set of neurons within a region, or distributed throughout the brain, that expresses a given molecule, or that is overactive in a given disease, or that responds to a given drug, or that projects from one region to another).

As used herein, a “subset” of a set consists of less than all of the elements of the set.

The term “such as” means for example.

A “supercapacitor” (or an “ultracapacitor”) means a capacitive energy storage device of at least 100 millifarads. For example, an electrolytic double-layer capacitor of at least 100 millifarads is a “supercapacitor”, as that term is used herein.

Spatially relative terms such as “under”, “below”, “above”, “over”, “upper”, “lower”, and the like, are used for ease of description to explain the positioning of one element relative to another. The terms are intended to encompass different orientations of an object in addition to different orientations than those depicted in the figures.

Except to the extent that the context clearly requires otherwise, if steps in a method are described herein, then: (1) steps in the method may occur in any order or sequence, even if the order or sequence is different than that described; (2) any step or steps in the method may occur more than once; (3) different steps, out of the steps in the method, may occur a different number of times during the method, (4) any step or steps in the method may be done in parallel or serially; (5) any step or steps in the method may be performed iteratively; (5) a given step in the method may be applied to the same thing each time that the particular step occurs or may be applied to different things each time that the given step occurs; and (6) the steps described are not an exhaustive listing of all of the steps in the method, and the method may include other steps.

This Definitions section shall, in all cases, control over and override any other definition of the Defined Terms. For example, the definitions of Defined Terms set forth in this Definitions section override common usage or any external dictionary. If a given term is explicitly or implicitly defined in this document, then that definition shall be controlling, and shall override any definition of the given term arising from any source (e.g., a dictionary or common usage) that is external to this document. If this document provides clarification regarding the meaning of a particular term, then that clarification shall, to the extent applicable, override any definition of the given term arising from any source (e.g., a dictionary or common usage) that is external to this document. To the extent that any term or phrase is defined or clarified herein, such definition or clarification applies to any grammatical variation of such term or phrase, taking into account the difference in grammatical form. For example, the grammatical variations include noun, verb, participle, adjective, or possessive forms, or different declensions, or different tenses. In each case described in this paragraph, Applicant is acting as Applicant's own lexicographer.

Variations:

This invention may be implemented in many different ways, in addition to those described above.

Here are some non-limiting examples of how this invention may be implemented:

This invention may be implemented as a method comprising, in combination: (a) using an antenna and circuitry to receive energy by wireless transmission; (b) using a supercapacitor to store at least a portion of the energy and to provide power to one or more light sources; and (c) using an array of optical fibers or light guides, to deliver light from one or more light sources to living tissue of a mammal, which tissue includes light-sensitive, heterologously expressed proteins; wherein all or or a portion of the array is implanted in the living mammal. Furthermore: (1) All or a portion of the light source may be implanted in the mammal. (2) The wireless transmission may comprise transcutaneous energy transfer. (3) The light source may be implanted in a cranium. (4) The one or more light sources may be partially or wholly located externally to the mammal and the array may be at least partially inserted into the mammal. (5) A DC/DC converter may reduce voltage of wirelessly-received energy, after rectification and before delivery to the supercapacitor. (6) A DC/DC converter circuit may deliver an output voltage over a range of capacitor voltages, which output voltage does not vary more than 15%, or may deliver an output current over a range of capacitor voltages, which output current does not vary more than 15%. (7) One or more processors may adaptively control light output from the one or light sources, based at least in part on an algorithm that models heat transfer in the tissue. (8) One or more processors may generate control signals to shutdown light delivery if an increase in tissue temperature exceeds a specified threshold. (9) The light may trigger a change in voltage potential in cells in the tissue, or in subcellular regions of the cells. (10) The light may trigger a change in pH in cells in the tissue, or in subcellular regions of the cells. (11) The proteins may comprise a proton pump. (12) A voltage potential of all or a portion of a cell may be increased or decreased, until the cell or portion of a cell is hyperpolarized. (13) The cell may be a neuron and the hyperpolarization may achieve neural silencing. (14) A plurality of light-activated proton pumps responsive to different wavelengths of light may be used to achieve multi-color neural silencing by the steps of: (a) expressing each light-activated proton pump in a different population of cells; and (b) illuminating the cells with different colors of light. (15) The tissue may include cells, and nucleic acid for expressing the proteins may be transfected into the cells. (16) The tissue may include cells, and nucleic acid for expressing the proteins may be brought into the cells by viral infection.

This invention may be implemented as an implant device comprising, in combination: (a) an antenna and circuitry for receiving energy by wireless transmission at a time when all or a portion of the implant device is implanted in a living organism; (b) one or more supercapacitors for storing a portion of the energy and for providing power to one or more light sources; and (c) the one or more light sources, for illuminating a neural target in an interior region of the organism to optogenetically activate or inactivate the neural target.

This invention may be implemented as a method comprising, in combination: (a) using an antenna and circuitry to receive energy by wireless transmission; (b) using a supercapacitor to store at least a portion of the energy and to provide power to one or more light sources; and (c) using an array of optical fibers or light guides to deliver light from the one or more light sources to living tissue of a mammal, which light is in a specific frequency band, and which living tissue includes cells that are optogenetically sensitized to light that is in the specific frequency band; wherein the light triggers a change in a function of the tissue. Furthermore, nucleic acids may be moved into the cells, such that the nucleic acids subsequently heterologously express light-sensitive proteins.

While exemplary implementations are disclosed, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also within the scope of the present invention. Numerous modifications may be made by one of ordinary skill in the art without departing from the scope of the invention.

What is claimed is:

1. A method comprising, in combination:
(a) using an antenna and circuitry to receive energy by wireless transmission;
(b) using a supercapacitor to store at least a portion of the energy and to provide power to one or more light sources; and
(c) using an array of optical fibers or light guides, to deliver light from one or more light sources to living tissue of a mammal, which tissue includes light-sensitive, heterologously expressed proteins;
wherein all or or a portion of the array is implanted in the living mammal.

2. The method of claim 1, wherein all or a portion of the light source is implanted in the mammal.

3. The method of claim 1, wherein the wireless transmission comprises transcutaneous energy transfer.

4. The method of claim 2, wherein the light source is implanted in a cranium.

5. The method of claim 1, wherein the one or more light sources are partially or wholly located externally to the mammal and the array is at least partially inserted into the mammal.

6. The method of claim 1, further comprising using a DC/DC converter to reduce voltage of wirelessly-received energy, after rectification and before delivery to the supercapacitor.

7. The method of claim 1, wherein the method further comprises using a DC/DC converter circuit for delivering an output voltage over a range of capacitor voltages, which output voltage does not vary more than 15%, or for delivering an output current over a range of capacitor voltages, which output current does not vary more than 15%.

8. The method of claim 1, wherein the method further comprises using one or more processors to adaptively control light output from the one or light sources, based at least in part on an algorithm that models heat transfer in the tissue.

9. The method of claim 1, wherein the method further comprises using one or more processors to generate control signals to shutdown light delivery if an increase in tissue temperature exceeds a specified threshold.

10. The method of claim 1, wherein the light triggers a change in voltage potential in cells in the tissue, or in subcellular regions of the cells.

11. The method of claim 1, wherein the light triggers a change in pH in cells in the tissue, or in subcellular regions of the cells.

12. The method of claim 1, wherein the proteins comprise a proton pump.

13. The method of claim 1, wherein the method further comprises the step of increasing or decreasing a voltage potential of all or a portion of a cell, until the cell or portion of a cell is hyperpolarized.

14. The method of claim 13, wherein the cell is a neuron and the hyperpolarization achieves neural silencing.

15. The method of claim 1, further comprising the step of using a plurality of light-activated proton pumps responsive to different wavelengths of light to achieve multi-color neural silencing by the steps of:
(a) expressing each light-activated proton pump in a different population of cells; and
(b) illuminating the cells with different colors of light.

16. The method of claim 1, wherein the tissue includes cells, and the method further comprises transfecting, into the cells, nucleic acid for expressing the proteins.

17. The method of claim 1, wherein the tissue includes cells, and the method further comprises bringing into the cells, by viral infection, nucleic acid for expressing the proteins.

18. An implant device comprising, in combination:
(a) an antenna and circuitry for receiving energy by wireless transmission at a time when all or a portion of the implant device is implanted in a living organism;
(b) one or more supercapacitors for storing a portion of the energy and for providing power to one or more light sources; and
(c) the one or more light sources, for illuminating a neural target in an interior region of the organism to optogenetically activate or inactivate the neural target.

19. A method comprising, in combination:
(a) using an antenna and circuitry to receive energy by wireless transmission;
(b) using a supercapacitor to store at least a portion of the energy and to provide power to one or more light sources; and
(c) using an array of optical fibers or light guides to deliver light from one or more light sources to living tissue of a mammal, which light is in a specific frequency band, and which living tissue includes cells that are optogenetically sensitized to light that is in the specific frequency band;
wherein the light triggers a change in a function of the tissue.

20. The method of claim 19, wherein the method further comprises moving nucleic acids into the cells, such that the nucleic acids subsequently heterologously express light-sensitive proteins.
